(19) United States
(12) Patent Application Publication
(10) Pub. No.: US 2026/0085063 A1
(43) Pub. Date: Mar. 26, 2026

(54) BENZAMIDE COMPOUNDS AND METHODS OF USE THEREOF

(71) Applicant: Rutgers, The State University of New Jersey, New Brunswick, NJ (US)

(72) Inventors: Jun Wang, New Brunswick, NJ (US); Bin Tan, New Brunswick, NJ (US); Prakash Daulat Jadhav, New Brunswick, NJ (US)

(21) Appl. No.: 19/304,124

(22) Filed: Aug. 19, 2025

Related U.S. Application Data
(60) Provisional application No. 63/685,127, filed on Aug. 20, 2024.

(51) Int. Cl. C07D 403/14; A61K 31/4155; A61K 31/427; A61K 31/438; A61K 31/454; A61K 31/496; A61K 31/4995; A61K 31/5377; A61K 31/541; A61P 31/14; C07D 231/12; C07D 401/14; C07D 403/10; C07D 405/14; C07D 413/14; C07D 417/10; C07D 471/10; C07D 487/04; C07D 487/08

(52) U.S. Cl. CPC C07D 403/14; A61K 31/4155; A61K 31/427; A61K 31/438; A61K 31/454; A61K 31/496; A61K 31/4995; A61K 31/5377; A61K 31/541; A61P 31/14; C07D 231/12; C07D 401/14; C07D 403/10; C07D 405/14; C07D 413/14; C07D 417/10; C07D 471/10; C07D 487/04; C07D 487/08

(57) ABSTRACT

The present disclosure relates, in part, to SARS-CoV-2 papain-like protease (PL^{pro}) inhibitor compounds of Formula (I), pharmaceutical compositions thereof, and methods of using the same for promoting an antiviral effect in a subject and/or treating, preventing, and/or ameliorating a viral infection in a subject:

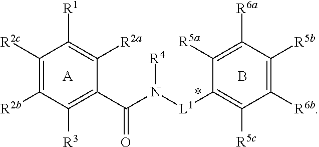

### CROSS-REFERENCE TO RELATED APPLICATIONS

This application claims priority under 35 U.S.C. § 119(e) to U.S. Provisional Patent Application No. 63/685,127, filed Aug. 20, 2024, which is incorporated herein by reference in its entirety.

### STATEMENT REGARDING FEDERALLY SPONSORED RESEARCH OR DEVELOPMENT

This invention was made with government support under U19AI171110 awarded by the National Institutes of Health. The government has certain rights in the invention.

### BACKGROUND

The COVID-19 pandemic is by far the worst pandemic since the 1918 Spanish flu. The etiological agent of COVID-19 is SARS-CoV-2, a single-stranded positive sense RNA virus that belongs to the beta coronavirus genus. Two additional coronaviruses within the same genus, SARS-CoV, and MERS-CoV, have caused epidemics in humans with mortality rates of 9.6% and 34.3%, respectively. Although SARS-CoV-2 has a lower mortality rate of 2.1% compared to SARS-CoV and MERS-CoV, it has led to a far greater death toll due to its higher transmission. SARS-CoV-2 differs from SARS-CoV and MERS-CoV in that it has a long incubation time after the initial infection (1 to 2 weeks), and a large percentage of infected patients continue to shed the virus while being asymptomatic, presenting a daunting task for surveillance and containment.

Three mRNA vaccines, which were developed by Pfizer/BioNTech, Moderna, and Johnson and Johnson, have been approved by FDA in the United States. For small molecule antivirals, remdesivir received FDA approval on Oct. 22, 2020. Although the polymerase of SARS-CoV-2 has proof-reading function, it continues to mutate at a rate about 10⁻⁶ per site per cycle. Several variants have already emerged and widely circulated among humans since the beginning of the pandemic. Therefore, there is a dire need for additional antivirals with a novel mechanism of action.

Antivirals are not substituents of vaccines, but rather an important complement that can be used for the treatment of infection from both wild-type (WT) and variant viruses. Among the viral proteins that have been actively pursued as SARS-CoV-2 antiviral drug targets, the main protease (M^{pro}) and papain-like protease (PL^{pro}) are the most promising ones. M^{pro} and PL^{pro} are involved in the proteolytic digestion of the viral polyproteins pp1a and pp1ab, yielding individual functional viral proteins for the replication complex formation. PL^{pro} cleaves at three sites with the recognition sequence “LXGG⬇XX” PL^{pro} has been shown to play additional roles in dysregulating host immune response and impairing the host type I interferon antiviral effect through its deubiquitinating and deISG15ylating (interferon-induced gene 15) activities, respectively. SARS-CoV-2 PL^{pro} cleaves ISG15 and polyubiquitin modifications from cellular proteins, and inhibition of PL^{pro} led to the accumulation of ISG15-conjugates and poly-ubiquitin-conjugates. While SARS-CoV PL^{pro} prefers ubiquitinated substrates, SARS-CoV-2 PL^{pro} prefers the ISGlyated proteins as substrates. PL^{pro} is part of a membrane anchored multi-domain protein named non-structural protein 3 (nsp-3), an essential component of the replicase-transcriptase complex. The pleiotropic roles of SARS-CoV-2 PL^{pro} make it a promising antiviral drug target. Substantial morbidity and mortality associated with COVID-19 infection is caused by cytokine storm, and suppressing host immune response using dexamethasone and baricitinib has been shown to provide therapeutic benefits in the treatment of severe infections.

Significant progress has been made in developing SARS-CoV-2 M^{pro} inhibitors. In comparison, PL^{pro} represents a more challenging drug target, and GRL0617 remains one of the most potent PL^{pro} inhibitors reported to date despite several high-throughput screening and medicinal chemistry optimization campaigns. GRL0617 was originally developed as a deubiquitinase inhibitor and was later identified as a SARS-CoV PL^{pro} inhibitor through a high-throughput screening. As SARS-CoV-2 and SARS-CoV PL^{pro} share a sequence identity of 83% and similarity of 90%, GRL0617 was also repurposed for SARS-CoV-2 PL^{pro} and it was reported to inhibit SARS-CoV-2 PL^{pro} with IC₅₀ values of around 2 μM and SARS-CoV-2 viral replication with EC₅₀ values around 20 μM from multiple studies.

There is thus a need in the art for SARS-CoV-2 antiviral agents that can be used alone or in combination with other drugs (e.g., protease inhibitors). The present disclosure addresses this need.

### BRIEF SUMMARY

In one aspect, the disclosure provides a compound of Formula (I), or a salt, stereoisomer, or isotopically labeled derivative thereof, wherein R¹, R^{2a}, R^{2b}, R^{2c}, R³, R⁴, R^{5a}, R^{5b}, R⁵, R^{6a}, R^{6b}, and L¹ are defined elsewhere herein:

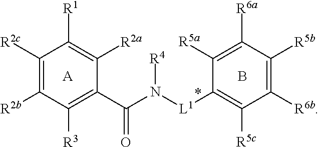

In one aspect, the disclosure provides a pharmaceutical composition comprising at least one compound of the disclosure and a pharmaceutically acceptable carrier.

In one aspect, the disclosure provides a method for promoting an antiviral effect in a subject. In certain embodiments, the method comprises administering at least one compound of the disclosure or a pharmaceutical composition thereof.

In one aspect, the disclosure provides a method for inhibiting a papain-like protease in a subject. In certain embodiments, the method comprises administering at least one compound of the disclosure or a pharmaceutical composition thereof.

In one aspect, the disclosure provides a method for treating, preventing, and/or ameliorating a viral infection in a subject. In certain embodiments, the method comprises administering at least one compound of the disclosure or a pharmaceutical composition thereof.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms.

Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical (e.g.,

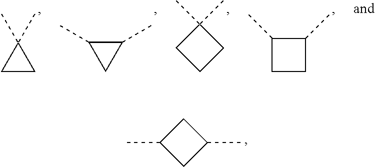

and inter alia). In certain embodiments, the term may be regarded as a product of removal of two hydrogen atoms from the corresponding cycloalkane (e.g., cyclobutyl) by removal of two hydrogen atoms from the same (e.g.,

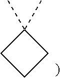

) different (e.g.,

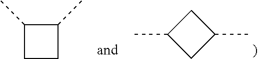

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂₋₁₄₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical (e.g.,

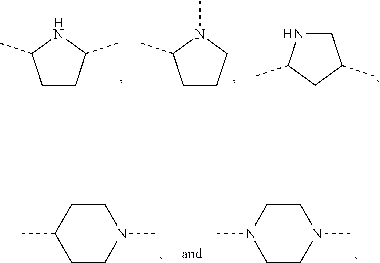

inter alia). In certain embodiments, the term may be regarded as a product of removal of two hydrogen atoms from the corresponding heterocycloalkane (e.g., piperidine) by removal of two hydrogen atoms from the same (e.g.,

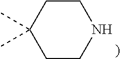

different (e.g.,

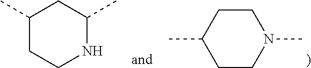

Carbon atom(s) and/or heteroatom(s). The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “linker” as used herein refers to an organic moiety that connects two parts of a compound (e.g., two small molecule drugs, or a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “phenylene” or “phenylenyl” as used herein refers to a bivalent phenyl radical (e.g., 1,4-phenylene). In certain embodiments, the term may be regarded as a divalent radical formed by the removal of two hydrogen atoms from a benzene moiety.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “specifically binds”, or “specifically binds”, or the like, means that a small molecule, an antibody, and/or antigen-binding fragment forms a complex with a target and/or an antigen that is relatively stable under physiological conditions. The specific bond can be characterized by an equilibrium dissociation constant (for example, a smaller K_{D} denotes a firmer bond). Methods for determining whether two molecules specifically bind to each other are well known in the art and include, for example, equilibrium dialysis, surface plasmon resonance, and the like.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Compounds

In one aspect, the disclosure provides a compound of Formula (I), or a salt, stereoisomer, or isotopically labeled derivative thereof:

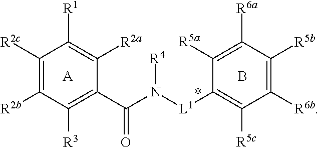

wherein:
R¹ is selected from the group consisting of

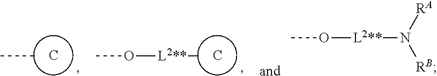

R^{2a}, R^{2b}, and R^{2c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R³ is CH₃;
R⁴ is H;
R^{5a}, R^{5b}, and R^{5c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{6a} is selected from the group consisting of:

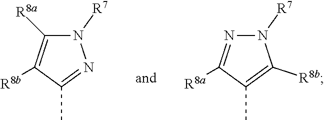

R^{6b} is selected from the group consisting of:

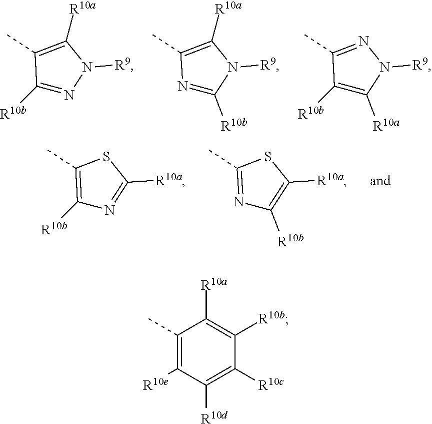

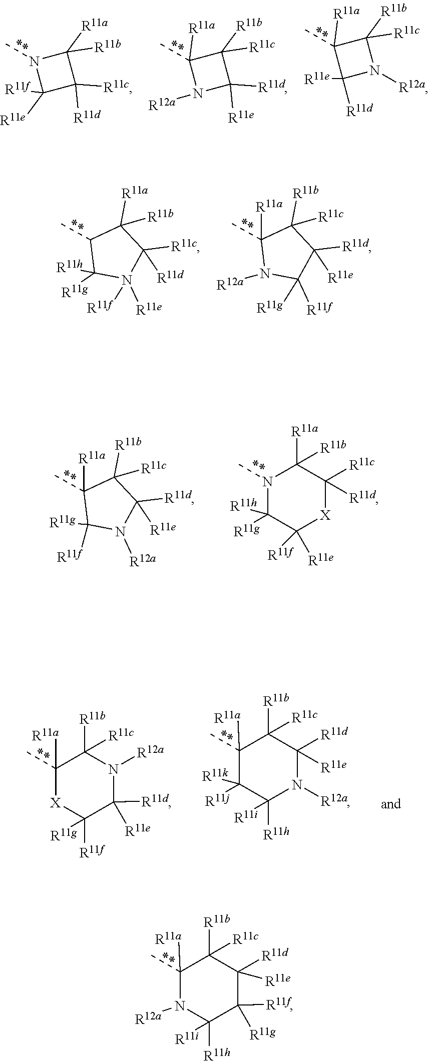

wherein ** indicates a bond between L² and ring C or N(R^{A})(R^{B});
L¹ is selected from the group consisting of

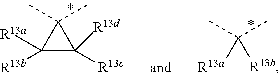

wherein * indicates a bond between L¹ and ring B;
L² is selected from the group consisting of -(optionally substituted C₁-C₆ alkylenyl)- and -(optionally substituted C₂-C₆ heteroalkylenyl)-;
R⁷ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{8a} and R^{8b} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R⁹ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{10a}, R^{10b}, R^{10c}, R^{10d} and R^{10e}, if present, are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{11a}, R^{11b}, R^{11c}, R^{11d}, R^{11e}, R^{11f}, R^{11g}, R^{11h}, R¹¹ⁱ, and R^{11j}, if present, are each independently selected from the group consisting of H, halogen, optionally substituted C₁-C₆ alkyl, and N(R^{A})(R^{B}), or
   two substituents selected from the group consisting of R^{11a}, R^{11b}, R^{11c}, R^{11d}, R^{11e}, R^{11f}, R^{11g}, R^{h11}, R¹¹ⁱ, and R^{11j} can combine with the atoms to which they are bound to form an optionally substituted C₃-C₈ heterocycloalkyl or optionally substituted C₃-C₈ cycloalkyl;
R^{12a} and R^{12b}, if present, are each independently selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{13a}, R^{13b}, R^{13c}, and R^{13d}, if present, are each independently selected from the group consisting of H and optionally substituted C₁-C₆ alkyl;
X is selected from the group consisting of —C(R¹¹ⁱ)(R^{11j})—, —N(R^{12b})—, —O—, and —S—;
each occurrence of R^{A} and R^{B}, if present, is independently selected from the group consisting of H, C(═O)R^{C}, C(═O)OR^{C}, C(═O)N(R^{C})(R^{D}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
R^{C} and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

In certain embodiments, the compound is not 2-methyl-N-(1-(3-(1-methyl-1H-pyrazol-4-yl)-5-(thiazol-5-yl)phenyl)cyclopropyl)-5-(piperazin-1-yl)benzamide.

In certain embodiments, L¹ is

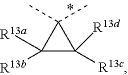

### R^{6b} is

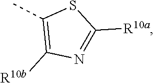

and no more than one of R^{10a} and R^{10b} is H, optionally wherein at least one of R^{10a} and R^{10b} is C₁-C₆ alkyl.

In certain embodiments, L¹ is

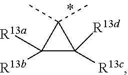

### R^{6b} is

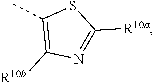

### and R^{6a} is

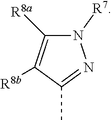

In certain embodiments, L¹ is

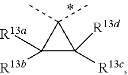

### R^{6b} is

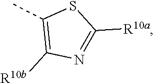

### and R^{6a} is

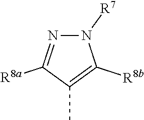

and R⁷ is C₂-C₆ alkyl.

In certain embodiments, L¹ is

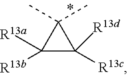

### R¹ is

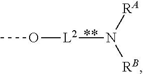

### R^{6a} is

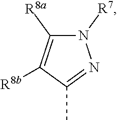

and R^{6b} is selected from the group consisting of consisting of

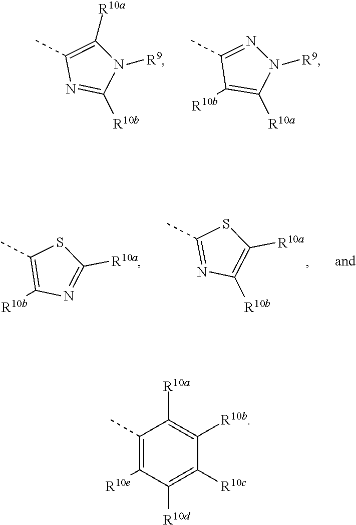

In certain embodiments, L¹ is

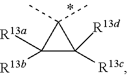

### R¹ is

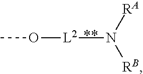

### R^{6a} is

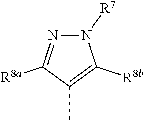

and R^{6b} is selected from the group consisting of

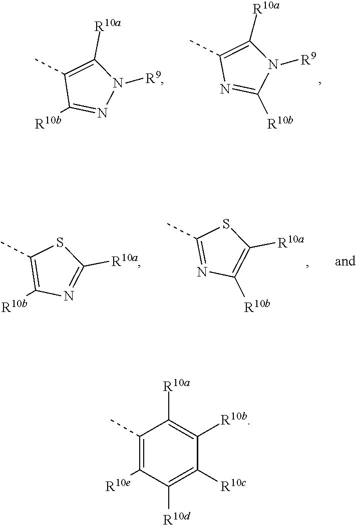

In certain embodiments, L¹ is

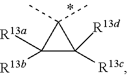

### R¹ is

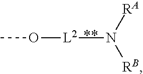

### R^{6a} is

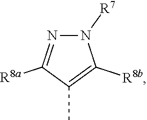

and N(R^{A})(R^{B}) is NEt₂.

In certain embodiments, L¹ is

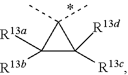

### R¹ is

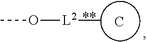

ring C is

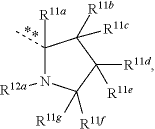

and at least one of R^{11a}, R^{11b}, R^{11c}, R^{11d}, R^{11e}, R^{11f}, or R^{11g} is F. In certain embodiments, L¹ is

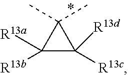

### R¹ is

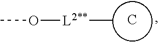

ring C is

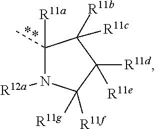

and the carbon atom substituted with R^{11a} has (S)-configuration.

In certain embodiments, L¹ is

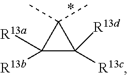

### R¹ is

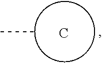

ring C is

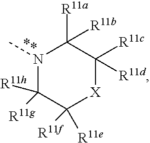

X is N(R^{12b}), and one of R^{2a}, R^{2b}, and R^{2c} is F.

In certain embodiments, L¹ is

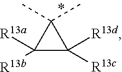

### R¹ is

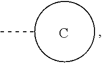

ring C is

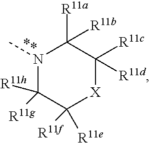

X is N(R^{12b}) and R^{12b} is C₂-C₆ alkyl or C₃-C₈ cycloalkyl.

In certain embodiments, L¹ is

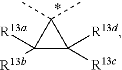

### R¹ is

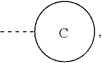

ring C is

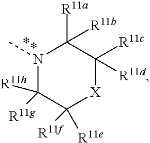

X is N(R^{12b}), and no more than three of R^{11c}, R^{11d}, R^{11e}, and R^{11f} are H.

In certain embodiments, L¹ is

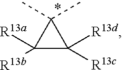

### R¹ is

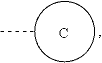

ring C is

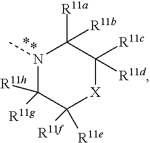

X is N(R^{12b}), and at least one of R⁷ or R⁹ is substituted C₁-C₆ alkyl, optionally substituted C₃-C₈ cycloalkyl, or optionally substituted C₂-C₈ heterocycloalkyl. In certain embodiments, L¹ is

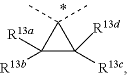

### R¹ is

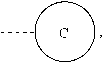

ring C is

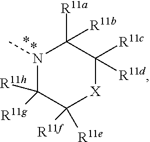

X is N(R^{12b}), and R^{6b} is

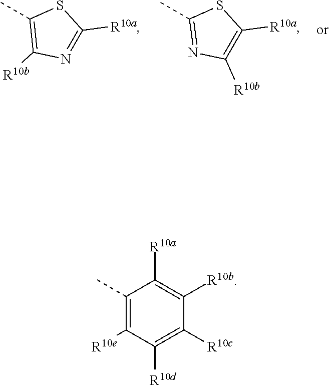

In certain embodiments, L¹ is

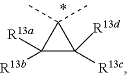

### R¹ is

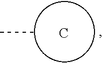

ring C is

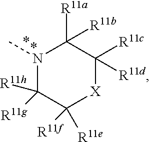

X is N(R^{12b}), and R⁷ and R⁹ are both CH₃ or CH₂CH₃.

In certain embodiments, L¹ is

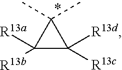

### R¹ is

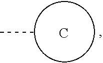

ring C is

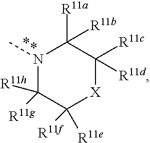

X is N(R^{12b}), one of R^{11a}, R^{11b}, R^{11h}, and R^{11g} is CH₃, and R⁷ and R⁹ are both CH₃.

In certain embodiments, L¹ is

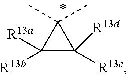

### R¹ is

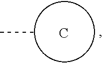

ring C is

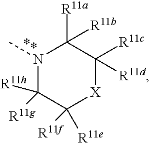

X is N(R^{12b}), one of R^{11a}, R^{11b}, R^{11h}, and R^{11g} is CH₃, and R^{6a} is

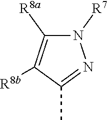

and R^{6b} is selected from the group consisting of

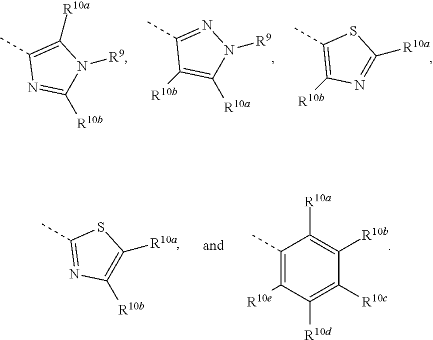

In certain embodiments, L¹ is

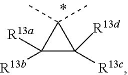

### R¹ is

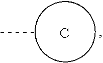

ring C is

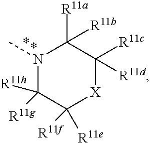

X is N(R^{12b}), one of R^{11a}, R^{11b}, R^{11h}, and R^{11g} is CH₃, and R^{6a} is

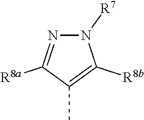

and R^{6b} is selected from the group consisting of

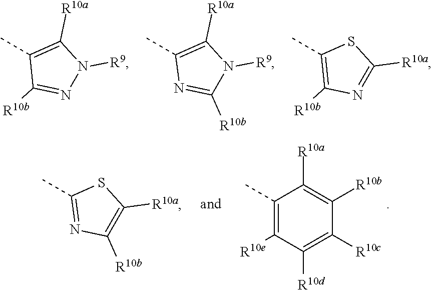

In certain embodiments, L¹ is

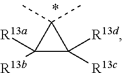

### R¹ is

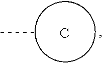

ring C is

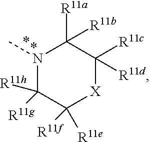

X is N(R^{12b}), and one substituent selected from the group consisting of R^{11a} and R^{11b} combines with one substituent selected from the group consisting of R^{11e} and R^{11f} to form a C₃-C₅ heterocycloalkyl.

In certain embodiments, L¹ is

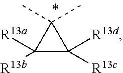

### R¹ is

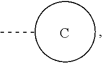

ring C is

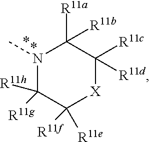

X is N(R^{12b}), one substituent selected from the group consisting of R^{11a}, R^{11b}, R^{11c} and R^{11d} combines with one substituent selected from the group consisting of R^{11e}, R^{11f}, R^{11g}, and R^{11h} to form a C₄-C₈ heterocycloalkyl, and R⁷ and R⁹ are both CH₃.

In certain embodiments, the compound of Formula (I) is a compound of Formula (Ia):

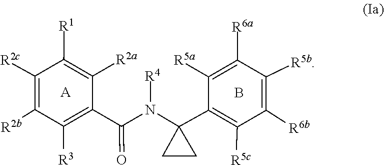

In certain embodiments, the compound of Formula (I) is a compound of Formula (Ib):

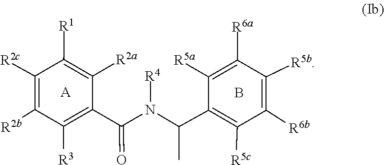

In certain embodiments, the compound of Formula (I) is a compound of Formula (Ib-1):

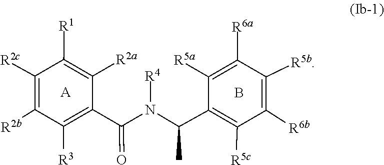

In certain embodiments, the compound of Formula (I) is a compound of Formula (Ib-2):

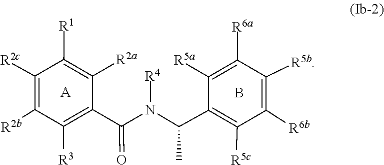

In certain embodiments, R^{11a}, R^{11b}, R^{11c}, R^{11d}, R^{11e}, R^{11f}, R^{11g}, R^{11h}, R¹¹ⁱ, and R^{11j}, if present, are each independently selected from the group consisting of H, F, and CH₃.

In certain embodiments, two substituents selected from the group consisting of R^{11a}, R^{11b}, R^{11c}, R^{11d}, R^{11e}, R^{11f}, R^{11g}, R^{11h}, R¹¹ⁱ, and R^{11j}, combine with the atoms to which they are bound to form an optionally substituted azetidine or optionally substituted pyrrolidine.

In certain embodiments, R^{12a} and R^{12b}, if present, are each independently selected from the group consisting of H, CH₃, CH(CH₃)₂, C(CH₃)₃, cyclopropyl, and cyclobutyl.

In certain embodiments, ring C is

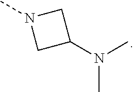

In certain embodiments, ring C is

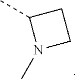

In certain embodiments, ring C is

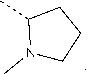

In certain embodiments, ring C is

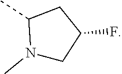

In certain embodiments, ring C is

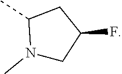

In certain embodiments, ring C is

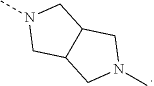

In certain embodiments, ring C is

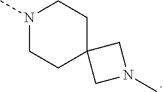

In certain embodiments, ring C is

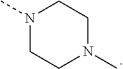

In certain embodiments, ring C is

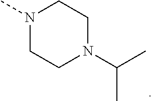

In certain embodiments, ring C is

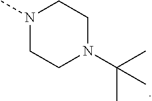

In certain embodiments, ring C is

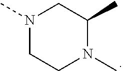

In certain embodiments, ring C is

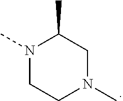

In certain embodiments, ring C is

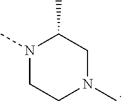

In certain embodiments, ring C is

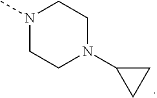

In certain embodiments, ring C is

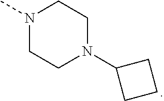

In certain embodiments, ring C is

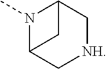

In certain embodiments, ring C is

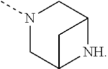

In certain embodiments, ring C is

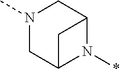

In certain embodiments, ring C is

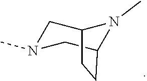

In certain embodiments, ring C is

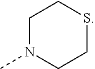

In certain embodiments, ring C is

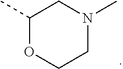

In certain embodiments, ring C is

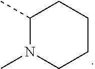

In certain embodiments, R¹ is

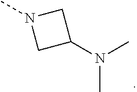

In certain embodiments, R¹ is

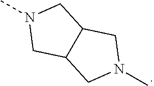

In certain embodiments, R¹ is

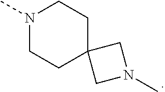

In certain embodiments, R¹ is

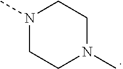

In certain embodiments, R¹ is

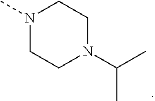

In certain embodiments, R¹ is

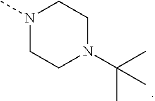

In certain embodiments, R¹ is

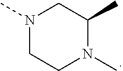

In certain embodiments, R¹ is

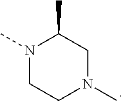

In certain embodiments, R¹ is

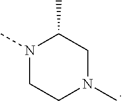

In certain embodiments, R¹ is

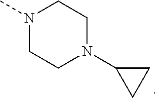

In certain embodiments, R¹ is

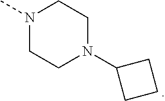

In certain embodiments, R¹ is

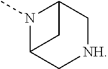

In certain embodiments, R¹ is

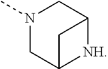

In certain embodiments, R¹ is

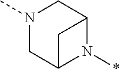

In certain embodiments, R¹ is

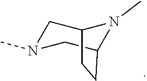

In certain embodiments, R¹ is

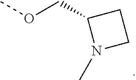

In certain embodiments, R¹ is

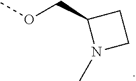

In certain embodiments, R¹ is

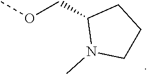

In certain embodiments, R¹ is

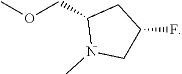

In certain embodiments, R¹ is

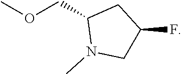

In certain embodiments, R¹ is

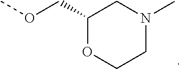

In certain embodiments, R¹ is

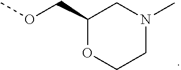

In certain embodiments, R¹ is

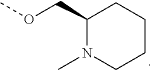

In certain embodiments, R¹ is

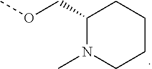

In certain embodiments, R¹ is

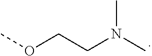

In certain embodiments, R¹ is

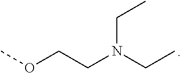

In certain embodiments, R¹ is

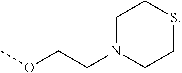

In certain embodiments, R^{2a} is H. In certain embodiments, R^{2a} is F. In certain embodiments, R^{2b} is H. In certain embodiments, R^{2b} is F. In certain embodiments, R^{2c} is H. In certain embodiments, R^{2c} is H.

In certain embodiments, R^{5a} is H. In certain embodiments, R^{5b} is H. In certain embodiments, R^{5c} is H.

In certain embodiments, R⁷ is CH₃. In certain embodiments, R⁷ is CF₂H. In certain embodiments, R⁷ is CH₂CH₃. In certain embodiments, R⁷ is CH(CH₃)₂. In certain embodiments, R⁷ is oxetanyl.

In certain embodiments, R^{8a} is H. In certain embodiments, R^{8b} is H.

In certain embodiments, R^{6a} is

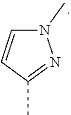

In certain embodiments, R^{6a} is

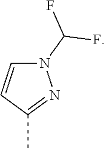

In certain embodiments, R^{6a} is

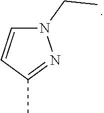

In certain embodiments, R^{6a} is

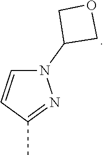

In certain embodiments, R^{6a} is

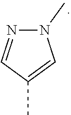

In certain embodiments, R^{6a} is

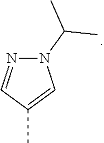

In certain embodiments, R⁹ is CH₃. In certain embodiments, R⁹ is CH₂CH₃. In certain embodiments, R⁹ is CF₂H. In certain embodiments, R⁹ is CH₂CN. In certain embodiments, R⁹ is CH₂OCH₃. In certain embodiments, R⁹ is CH₂CF₂H. In certain embodiments, R⁹ is CH₂CF₃, CH₂(cyclopropyl). In certain embodiments, R⁹ is (CH₂)₂F. In certain embodiments, R⁹ is (CH₂)₂CN. In certain embodiments, R⁹ is (CH₂)₂₀H. In certain embodiments, R⁹ is (CH₂)₂OCH₃. In certain embodiments, R⁹ is cyclopropyl. In certain embodiments, R⁹ is oxetanyl.

In certain embodiments, R^{10a} is H. In certain embodiments, R^{10a} is CH₃. In certain embodiments, R^{10a} is O(CH₂)₂O(CH₂)₂OCH₃. In certain embodiments, R^{10b} is H. In certain embodiments, R^{10b} is CH₃. In certain embodiments, R^{10b} is O(CH₂)₂O(CH₂)₂OCH₃. In certain embodiments, R^{10c} is H. In certain embodiments, R^{10c} is CH₃. In certain embodiments, R^{10c} is O(CH₂)₂O(CH₂)₂OCH₃. In certain embodiments, R^{10d} is H. In certain embodiments, R^{10d} is CH₃.

In certain embodiments, R^{10d} is O(CH₂)₂O(CH₂)₂OCH₃. In certain embodiments, R^{10e} is H. In certain embodiments, R^{10e} is CH₃. In certain embodiments, R^{10e} is O(CH₂)₂O(CH₂)₂OCH₃.

In certain embodiments, R^{6b} is N

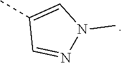

In certain embodiments, R^{6b} is

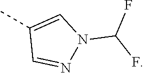

In certain embodiments, R^{6b} is

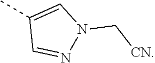

In certain embodiments, R^{6b} is N

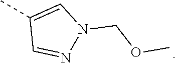

In certain embodiments, R^{6b} is

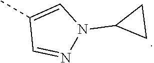

In certain embodiments, R^{6b} is

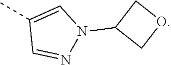

In certain embodiments, R^{6b} is

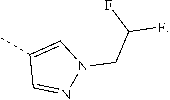

In certain embodiments, R^{6b} is

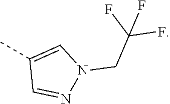

In certain embodiments, R^{6b} is

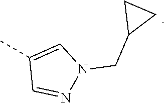

In certain embodiments, R^{6b} is

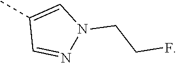

certain embodiments, R^{6b} is

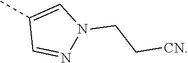

In certain embodiments, R^{6b} is

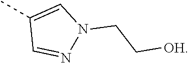

In certain embodiments, R^{6b} b is

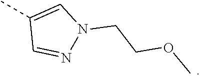

In certain embodiments, R^{6b} is

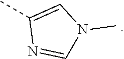

In certain embodiments, R^{6b} is

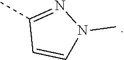

In certain embodiments, R^{6b} is

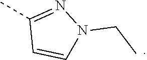

In certain embodiments, R^{6b} is

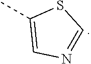

In certain embodiments, R^{6b} is

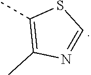

In certain embodiments, R^{6b} is

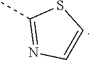

In certain embodiments, R^{6b} is

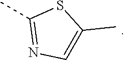

In certain embodiments, R^{6b} is

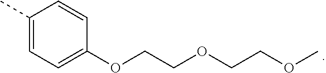

In certain embodiments, the compound is selected from the group consisting of:
N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide
N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
5-(3-(dimethylamino)azetidin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-H-pyrazol-4-yl)phenyl)cyclopropyl)-5-((4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4S)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4R)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2R,4R)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2R,4S)-4-fluoro-1-methylpyrrolidin-2-yl)methoxy)-2-methylbenzamide;
N-(1-(3-(1-ethyl-1H-pyrazol-3-yl)-5-(1-methyl-H-pyrazol-4-yl)phenyl)cyclopropyl)-2-methyl-5-(2-methyl-2,7-diazaspiro[3.5]nonan-7-yl)benzamide;
N-(1-(3-(1-ethyl-1H-pyrazol-3-yl)-5-(1-methyl-1H-pyrazol-4-yl)phenyl)cyclopropyl)-2-methyl-5-(5-methylhexahydropyrrolo[3,4-c]pyrrol-2(1H)-yl)benzamide;
4-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-4-fluoro-2-methyl-5-(4-methylpiperazin-1-yl)benzamide;
3-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-H-pyrazol-4-yl)phenyl)cyclopropyl)-3-fluoro-2-methyl-5-(4-methylpiperazin-1-yl)benzamide;
5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-fluoro-6-methyl-3-(4-methylpiperazin-1-yl)benzamide;
5-(4-(tert-butyl)piperazin-1-yl)-N-(1-(3-(1-ethyl-1H-pyrazol-3-yl)-5-(1-methyl-1H-pyrazol-4-yl)phenyl)cyclopropyl)-2-methylbenzamide;
5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
N-(1-(3-(1-(cyclopropylmethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-ethyl-1H-pyrazol-3-yl)phenyl)cyclopropyl)-2-methyl-5-(4-methylpiperazin-1-yl)benzamide;
N-(1-(3-(1-(2,2-difluoroethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-cyclopropyl-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2,2,2-trifluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(thiazol-5-yl)phenyl)cyclopropyl)-2-methyl-5-(4-methylpiperazin-1-yl)benzamide;
5-(4-cyclobutylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-(2-thiomorpholinoethoxy)benzamide;
N-(1-(3,5-bis(1-ethyl-1H-pyrazol-3-yl)phenyl)cyclopropyl)-2-methyl-5-((1-methylazetidin-2-yl)methoxy)benzamide;
(S)-N-(1-(3,5-bis(1-ethyl-1H-pyrazol-3-yl)phenyl)cyclopropyl)-2-methyl-5-((1-methylazetidin-2-yl)methoxy)benzamide;
(R)-N-(1-(3,5-bis(1-ethyl-1H-pyrazol-3-yl)phenyl)cyclopropyl)-2-methyl-5-((1-methylazetidin-2-yl)methoxy)benzamide;
N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(2-fluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(4-methylthiazol-5-yl)phenyl)cyclopropyl)-2-methyl-5-(4-methylpiperazin-1-yl)benzamide;
N-(1-(3-(1-ethyl-1H-pyrazol-3-yl)-5-(thiazol-2-yl)phenyl)cyclopropyl)-2-methyl-5-(4-methylpiperazin-1-yl)benzamide;
N-(1-(3-(1-ethyl-1H-pyrazol-3-yl)-5-(5-methylthiazol-2-yl)phenyl)cyclopropyl)-2-methyl-5-(4-methylpiperazin-1-yl)benzamide;
5-(2-(dimethylamino)ethoxy)-N-(1-(3-(1-ethyl-1H-pyrazol-3-yl)-5-(thiazol-5-yl)phenyl)cyclopropyl)-2-methylbenzamide;
5-(2-(dimethylamino)ethoxy)-N-(1-(3-(1-ethyl-1H-pyrazol-3-yl)-5-(thiazol-2-yl)phenyl)cyclopropyl)-2-methylbenzamide;
N-(1-(3-(1-(2-cyanoethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2-hydroxyethyl)-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(1-methyl-1H-pyrazol-4-yl)phenyl)cyclopropyl)-5-(4-methylpiperazin-1-yl)benzamide;
5-(2-(diethylamino)ethoxy)-N-(1-(3-(1-ethyl-1H-pyrazol-3-yl)-5-(1-methyl-1H-pyrazol-4-yl)phenyl)cyclopropyl)-2-methylbenzamide;
5-(3-(dimethylamino)azetidin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-ethyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(4-methylpiperazin-1-yl)benzamide;
5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(R)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(S)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
N-(1-(3-(1-ethyl-1H-pyrazol-3-yl)-5-(1-(oxetan-3-yl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((S)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((R)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-(1-(3-(1-ethyl-1H-pyrazol-3-yl)-5-(1-(2-methoxyethyl)-1H-pyrazol-4-yl)phenyl)cyclopropyl)-2-methyl-5-(4-methylpiperazin-1-yl)benzamide;
N-(1-(3-(1-(cyanomethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-methyl-1H-pyrazol-4-yl)phenyl)cyclopropyl)-2-methyl-5-(4-methylpiperazin-1-yl)benzamide;
N-(1-(3-(1-isopropyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-imidazol-4-yl)phenyl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-5-yl)phenyl)cyclopropyl)-5-(4-methylpiperazin-1-yl)benzamide;
5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(R)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(S)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
N-(1-(3-(1-(2-fluoroethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-2-yl)phenyl)cyclopropyl)-5-(4-methylpiperazin-1-yl)benzamide;
5-(3,6-diazabicyclo[3.1.1]heptan-3-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)cyclopropyl)-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-4-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-4-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
N-(1-(3,5-bis(1-methyl-1H-pyrazol-4-yl)phenyl)cyclopropyl)-2-methyl-5-((1-methylazetidin-2-yl)methoxy)benzamide;
(S)-N-(1-(3,5-bis(1-methyl-1H-pyrazol-4-yl)phenyl)cyclopropyl)-2-methyl-5-((1-methylazetidin-2-yl)methoxy)benzamide;
(R)-N-(1-(3,5-bis(1-methyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(oxetan-3-yl)-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-2-methyl-5-(3-methyl-3,6-diazabicyclo[3.1.1]heptan-6-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
N-(1-(3-(1-ethyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-(methoxymethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-((R)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((R)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1R,5S)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(5-methylthiazol-2-yl)phenyl)cyclopropyl)-5-(4-methylpiperazin-1-yl)benzamide;
2-methyl-N-(1-(3-(1-methyl-1H-pyrazol-3-yl)-5-(4-methylthiazol-5-yl)phenyl)cyclopropyl)-5-(4-methylpiperazin-1-yl)benzamide;
5-(3,6-diazabicyclo[3.1.1]heptan-6-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(R)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(S)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
2-methyl-N-(1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((R)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((S)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
N-(1-(4′-(2-(2-methoxyethoxy)ethoxy)-5-(1-methyl-1H-pyrazol-3-yl)-[1,1′-biphenyl]-3-yl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-(oxetan-3-yl)-1H-pyrazol-4-yl)phenyl)cyclopropyl)-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(6-methyl-3,6-diazabicyclo[3.1.1]heptan-3-yl)benzamide; and
N-(1-(3-(1-(2-methoxyethyl)-1H-pyrazol-4-yl)-5-(1-methyl-1H-pyrazol-3-yl)phenyl)cyclopropyl)-2-methyl-5-(4-methylpiperazin-1-yl)benzamide.

TABLE 1
Exemplary compounds of the disclosure

| No. | Compound |
|---|---|
| 1 | 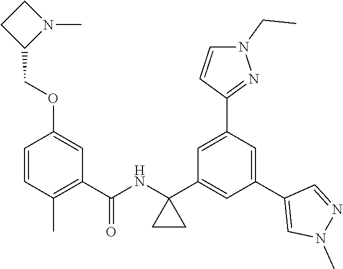 |
| | (S)-N-(1-(3-(1-ethyl-1H-pyrazol-3-yl)-5-(1-methyl-1H- |
| | pyrazol-4-yl)phenyl)cyclopropyl)-2-methyl-5- |
| | ((1-methylazetidin-2-yl)methoxy)benzamide |
| 2 | 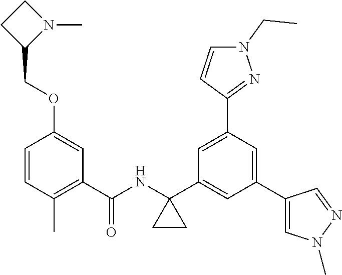 |
| | (R)-N-(1-(3-(1-ethyl-1H-pyrazol-3-yl)-5-(1-methyl- |
| | 1H-pyrazol-4-yl)phenyl)cyclopropyl)-2-methyl-5-((1- |
| | methylazetidin-2-yl)methoxy)benzamide |
| 3 | 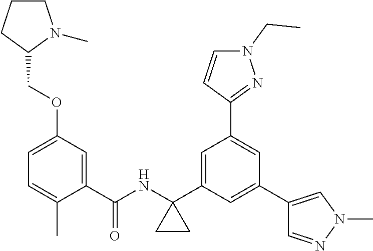 |
| | (S)-N-(1-(3-(1-ethyl-1H-pyrazol-3-yl)-5-(1-methyl-1H- |
| | pyrazol-4-yl)phenyl)cyclopropyl)-2-methyl-5-((1-methylpyrrolidin- |
| | 2-yl)methoxy)benzamide |
| 4 | 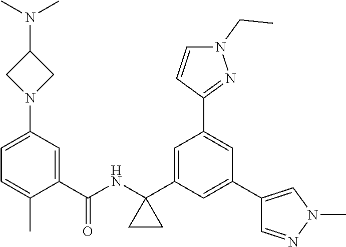 [SMILES: CCn1ccc(-c2cc(-c3cnn(C)c3)cc(C3(NC(=O)c4cc(N5CC(N(C)C)C5)ccc4C)CC3)c2)n1] |
| | 5-(3-(dimethylamino)azetidin-1-yl)-N-(1-(3- |
| | (1-ethyl-1H-pyrazol-3-yl)-5-(1-methyl- |
| | 1H-pyrazol-4-yl)phenyl)cyclopropyl)-2-methylbenzamide |
| 5 | 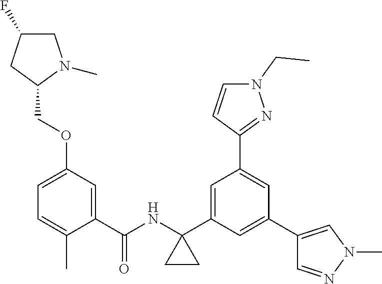 [SMILES: CCn1ccc(-c2cc(-c3cnn(C)c3)cc(C3(NC(=O)c4cc(OC[C@@H]5C[C@H](F)CN5C)ccc4C)CC3)c2)n1] |
| | N-(1-(3-(1-ethyl-1H-pyrazol-3-yl)-5-(1-methyl- |
| | 1H-pyrazol-4-yl)phenyl)cyclopropyl)-5-(((2S,4S)- |
| | 4-fluoro-1-methylpyrrolidin-2-yl)methoxy)-2-methylbenzamide |
| 6 | 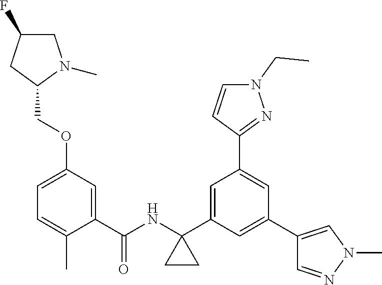 [SMILES: CCn1ccc(-c2cc(-c3cnn(C)c3)cc(C3(NC(=O)c4cc(OC[C@@H]5C[C@@H](F)CN5C)ccc4C)CC3)c2)n1] |
| | N-(1-(3-(1-ethyl-1H-pyrazol-3-yl)-5-(1-methyl-1H-pyrazol- |
| | 4-yl)phenyl)cyclopropyl)-5-(((2S,4R)-4-fluoro-1- |
| | methylpyrrolidin-2-yl)methoxy)-2-methylbenzamide |
| 7 | 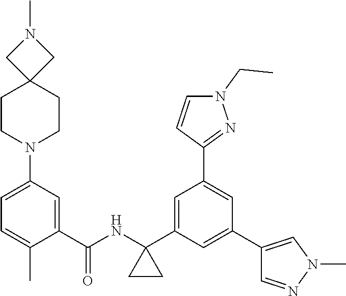 [SMILES: CCn1ccc(-c2cc(-c3cnn(C)c3)cc(C3(NC(=O)c4cc(N5CCC6(CC5)CN(C)C6)ccc4C)CC3)c2)n1] |
| | N-(1-(3-(1-ethyl-1H-pyrazol-3-yl)-5-(1-methyl- |
| | 1H-pyrazol-4-yl)phenyl)cyclopropyl)-2-methyl-5- |
| | (2-methyl-2,7-diazaspiro[3.5]nonan-7-yl)benzamide |
| 8 | 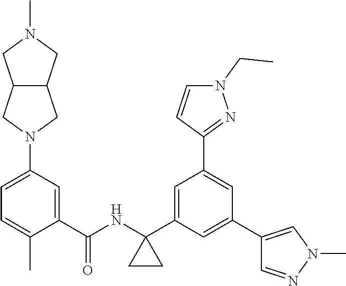 [SMILES: CCn1ccc(-c2cc(-c3cnn(C)c3)cc(C3(NC(=O)c4cc(N5CC6CN(C)CC6C5)ccc4C)CC3)c2)n1] |
| | N-(1-(3-(1-ethyl-1H-pyrazol-3-yl)-5-(1-methyl-1H-pyrazol-4- |
| | yl)phenyl)cyclopropyl)-2-methyl-5-(5- |
| | methylhexahydropyrrolo[3,4-c]pyrrol-2(1H)-yl)benzamide |
| 9 | 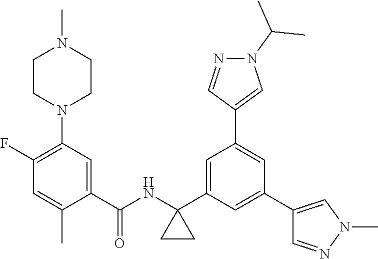 [SMILES: Cc1cc(F)c(N2CCN(C)CC2)cc1C(=O)NC1(c2cc(-c3cnn(C)c3)cc(-c3cnn(C(C)C)c3)c2)CC1] |
| | 4-fluoro-N-(1-(3-(1-isopropyl-1H-pyrazol-4-yl)- |
| | 5-(1-methyl-1H-pyrazol-4-yl)phenyl)cyclopropyl)- |
| | 2-methyl-5-(4-methylpiperazin-1-yl)benzamide |
| 10 | 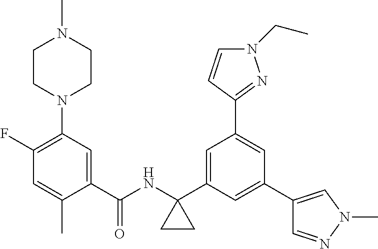 [SMILES: CCn1ccc(-c2cc(-c3cnn(C)c3)cc(C3(NC(=O)c4cc(N5CCN(C)CC5)c(F)cc4C)CC3)c2)n1] |
| | N-(1-(3-(1-ethyl-1H-pyrazol-3-yl)-5-(1-methyl- |
| | 1H-pyrazol-4-yl)phenyl)cyclopropyl)- |
| | 4-fluoro-2-methyl-5-(4-methylpiperazin-1-yl)benzamide |
| 11 | 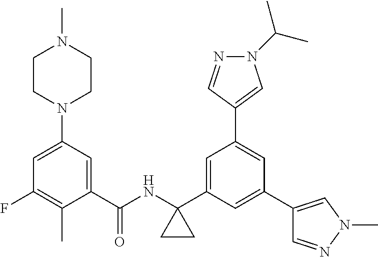 [SMILES: Cc1c(F)cc(N2CCN(C)CC2)cc1C(=O)NC1(c2cc(-c3cnn(C)c3)cc(-c3cnn(C(C)C)c3)c2)CC1] |
| | 3-fluoro-N-(1-(3-(1-isopropyl-1H-pyrazol-4-yl)- |
| | 5-(1-methyl-1H-pyrazol-4-yl)phenyl)cyclopropyl)- |
| | 2-methyl-5-(4-methylpiperazin-1-yl)benzamide |
| 12 | 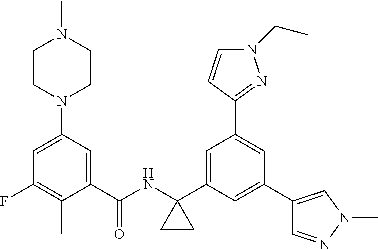 [SMILES: CCn1ccc(-c2cc(-c3cnn(C)c3)cc(C3(NC(=O)c4cc(N5CCN(C)CC5)cc(F)c4C)CC3)c2)n1] |
| | N-(1-(3-(1-ethyl-1H-pyrazol-3-yl)-5-(1-methyl- |
| | 1H-pyrazol-4-yl)phenyl)cyclopropyl)- |
| | 3-fluoro-2-methyl-5-(4-methylpiperazin-1-yl)benzamide |
| 13 | 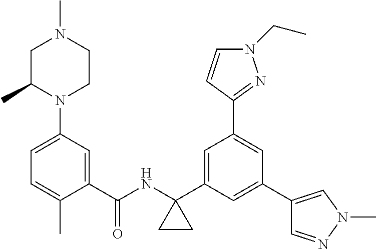 |
| | (S)-5-(2,4-dimethylpiperazin-1-yl)-N-(1-(3-(1- |
| | ethyl-1H-pyrazol-3-yl)-5-(1-methyl-1H- |
| | pyrazol-4-yl)phenyl)cyclopropyl)-2-methylbenzamide |
| 14 | 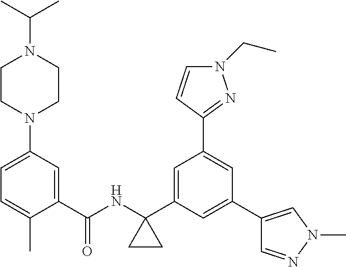 [SMILES: CCn1ccc(-c2cc(-c3cnn(C)c3)cc(C3(NC(=O)c4cc(N5CCN(C(C)C)CC5)ccc4C)CC3)c2)n1] |
| | N-(1-(3-(1-ethyl-1H-pyrazol-3-yl)-5-(1-methyl-1H- |
| | pyrazol-4-yl)phenyl)cyclopropyl)- |
| | 5-(4-isopropylpiperazin-1-yl)-2-methylbenzamide |
| 15 | 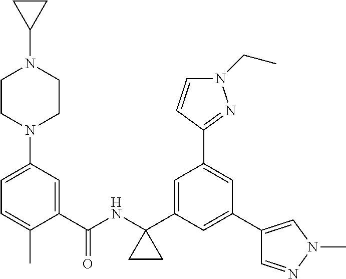 [SMILES: CCn1ccc(-c2cc(-c3cnn(C)c3)cc(C3(NC(=O)c4cc(N5CCN(C6CC6)CC5)ccc4C)CC3)c2)n1] |
| | 5-(4-cyclopropylpiperazin-1-yl)-N-(1-(3-(1-ethyl-1H- |
| | pyrazol-3-yl)-5-(1-methyl-1H- |
| | pyrazol-4-yl)phenyl)cyclopropyl)-2-methylbenzamide |
| 16 | 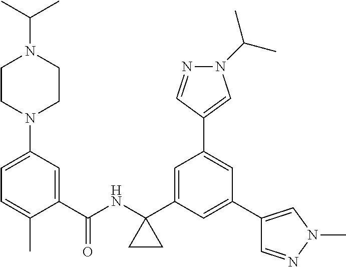 [SMILES: Cc1ccc(N2CCN(C(C)C)CC2)cc1C(=O)NC1(c2cc(-c3cnn(C)c3)cc(-c3cnn(C(C)C)c3)c2)CC1] |
| | N-(1-(3-(1-isopropyl-1H-pyrazol-4-yl)-5-(1- |
| | methyl-1H-pyrazol-4-yl)phenyl)cyclopropyl)- |
| | 5-(4-isopropylpiperazin-1-yl)-2-methylbenzamide |
| 17 | 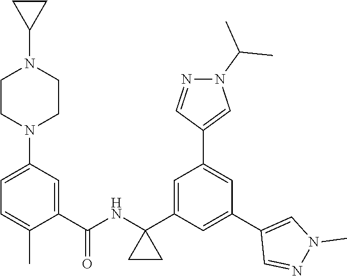 [SMILES: Cc1ccc(N2CCN(C3CC3)CC2)cc1C(=O)NC1(c2cc(-c3cnn(C)c3)cc(-c3cnn(C(C)C)c3)c2)CC1] |
| | 5-(4-cyclopropylpiperazin-1-yl)-N-(1-(3-(1- |
| | isopropyl-1H-pyrazol-4-yl)-5-(1-methyl-1H-pyrazol- |
| | 4-yl)phenyl)cyclopropyl)-2-methylbenzamide |
| 18 | 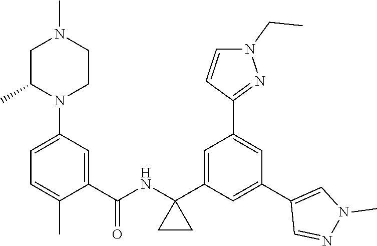 |
| | (R)-5-(2,4-dimethylpiperazin-1-yl)-N-(1-(3-(1-ethyl- |
| | 1H-pyrazol-3-yl)-5-(1-methyl-1H-pyrazol-4-yl) |
| | phenyl)cyclopropyl)-2-methylbenzamide |
| 19 | 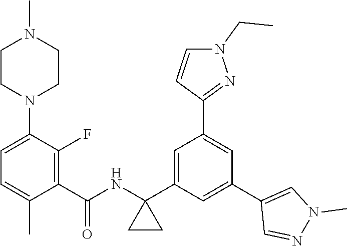 [SMILES: CCn1ccc(-c2cc(-c3cnn(C)c3)cc(C3(NC(=O)c4c(C)ccc(N5CCN(C)CC5)c4F)CC3)c2)n1] |
| | N-(1-(3-(1-ethyl-1H-pyrazol-3-yl)-5-(1-methyl-1H- |
| | pyrazol-4-yl)phenyl)cyclopropyl)- |
| | 2-fluoro-6-methyl-3-(4-methylpiperazin-1-yl)benzamide |
| 20 | 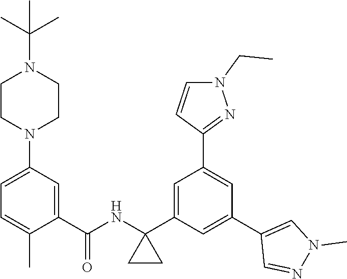 [SMILES: CCn1ccc(-c2cc(-c3cnn(C)c3)cc(C3(NC(=O)c4cc(N5CCN(C(C)(C)C)CC5)ccc4C)CC3)c2)n1] |
| | 5-(4-(tert-butyl)piperazin-1-yl)-N-(1-(3- |
| | (1-ethyl-1H-pyrazol-3-yl)-5-(1-methyl-1H- |
| | pyrazol-4-yl)phenyl)cyclopropyl)-2-methylbenzamide |
| 21 | 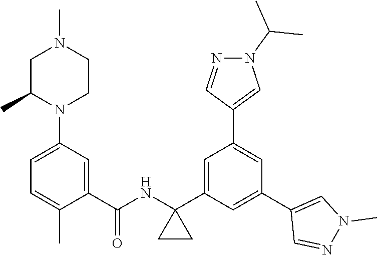 |
| | (S)-5-(2,4-dimethylpiperazin-1-yl)-N-(1-(3-(1- |
| | isopropyl-1H-pyrazol-4-yl)-5-(1-methyl-1H- |
| | pyrazol-4-yl)phenyl)cyclopropyl)-2-methylbenzamide |
| 22 | 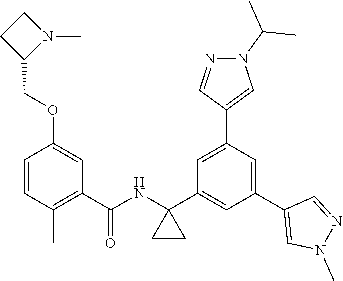 |
| | (S)-N-(1-(3-(1-isopropyl-1H-pyrazol-4-yl)-5-(1- |
| | methyl-1H-pyrazol-4-yl)phenyl)cyclopropyl)- |
| | 2-methyl-5-((1-methylazetidin-2-yl)methoxy)benzamide |
| 23 | 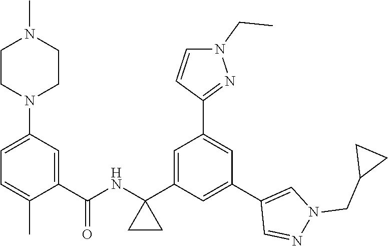 [SMILES: CCn1ccc(-c2cc(-c3cnn(CC4CC4)c3)cc(C3(NC(=O)c4cc(N5CCN(C)CC5)ccc4C)CC3)c2)n1] |
| | N-(1-(3-(1-(cyclopropylmethyl)-1H-pyrazol-4- |
| | yl)-5-(1-ethyl-1H-pyrazol-3-yl)phenyl)cyclopropyl)- |
| | 2-methyl-5-(4-methylpiperazin-1-yl)benzamide |
| 24 | 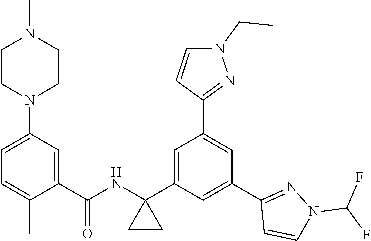 [SMILES: CCn1ccc(-c2cc(-c3ccn(C(F)F)n3)cc(C3(NC(=O)c4cc(N5CCN(C)CC5)ccc4C)CC3)c2)n1] |
| | N-(1-(3-(1-(difluoromethyl)-1H-pyrazol-3-yl)- |
| | 5-(1-ethyl-1H-pyrazol-3-yl)phenyl)cyclopropyl)- |
| | 2-methyl-5-(4-methylpiperazin-1-yl)benzamide |
| 25 | 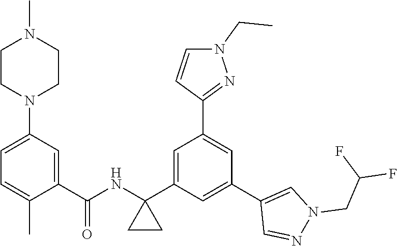 [SMILES: CCn1ccc(-c2cc(-c3cnn(CC(F)F)c3)cc(C3(NC(=O)c4cc(N5CCN(C)CC5)ccc4C)CC3)c2)n1] |
| | N-(1-(3-(1-(2,2-difluoroethyl)-1H-pyrazol-4-yl)-5- |
| | (1-ethyl-1H-pyrazol-3-yl)phenyl)cyclopropyl)- |
| | 2-methyl-5-(4-methylpiperazin-1-yl)benzamide |
| 26 | 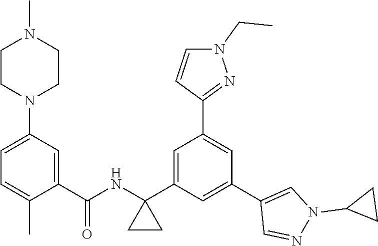 [SMILES: CCn1ccc(-c2cc(-c3cnn(C4CC4)c3)cc(C3(NC(=O)c4cc(N5CCN(C)CC5)ccc4C)CC3)c2)n1] |
| | N-(1-(3-(1-cyclopropyl-1H-pyrazol-4-yl)-5-(1-ethyl- |
| | 1H-pyrazol-3-yl)phenyl)cyclopropyl)- |
| | 2-methyl-5-(4-methylpiperazin-1-yl)benzamide |
| 27 | 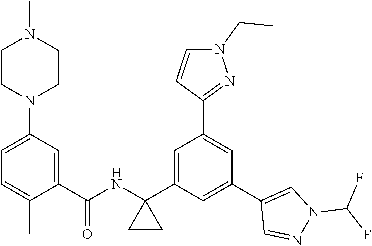 [SMILES: CCn1ccc(-c2cc(-c3cnn(C(F)F)c3)cc(C3(NC(=O)c4cc(N5CCN(C)CC5)ccc4C)CC3)c2)n1] |
| | N-(1-(3-(1-(difluoromethyl)-1H-pyrazol-4-yl)-5- |
| | (1-ethyl-1H-pyrazol-3-yl)phenyl)cyclopropyl)- |
| | 2-methyl-5-(4-methylpiperazin-1-yl)benzamide |
| 28 | 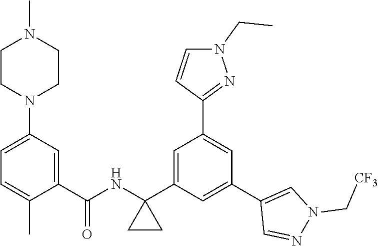 [SMILES: CCn1ccc(-c2cc(-c3cnn(CC(F)(F)F)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(2,2,2- |
| | trifluoroethyl)-1H-pyrazol-4-yl)phenyl)cyclopropyl)- |
| | 2-methyl-5-(4-methylpiperazin-1-yl)benzamide |
| 29 | 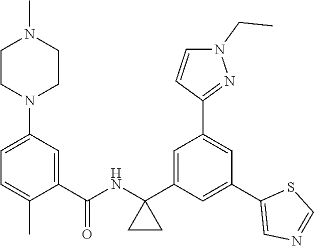 [SMILES: CCn1ccc(-c2cc(-c3cncs3)cc(C3(NC(=O)c4cc(N5CCN(C)CC5)ccc4C)CC3)c2)n1] |
| | N-(1-(3-(1-ethyl-1H-pyrazol-3-yl)-5-(thiazol-5-yl)phenyl) |
| | cyclopropyl)-2-methyl-5-(4-methylpiperazin-1-yl) |
| | benzamide |
| 30 | 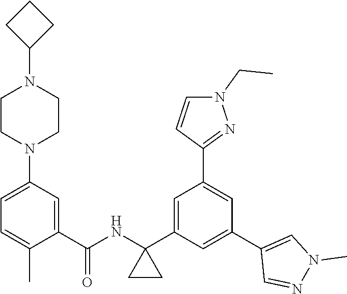 [SMILES: CCn1ccc(-c2cc(-c3cnn(C)c3)cc(C3(NC(=O)c4cc(N5CCN(C6CCC6)CC5)ccc4C)CC3)c2)n1] |
| | 5-(4-cyclobutylpiperazin-1-yl)-N-(1-(3-(1-ethyl-1H- |
| | pyrazol-3-yl)-5-(1-methyl-1H-pyrazol-4- |
| | yl)phenyl)cyclopropyl)-2-methylbenzamide |
| 31 | 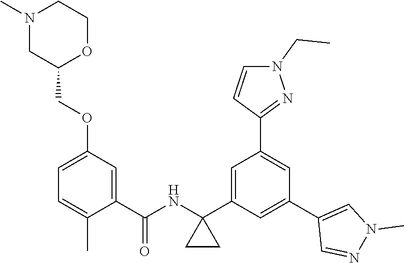 |
| | (S)-N-(1-(3-(1-ethyl-1H-pyrazol-3-yl)-5-(1-methyl-1H-pyrazol-4- |
| | yl)phenyl)cyclopropyl)-2-methyl-5-((4-methylmorpholin- |
| | 2-yl)methoxy)benzamide |
| 32 | 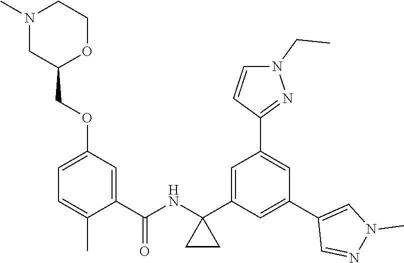 |
| | (R)-N-(1-(3-(1-ethyl-1H-pyrazol-3-yl)-5-(1-methyl-1H-pyrazol-4- |
| | yl)phenyl)cyclopropyl)-2-methyl-5-((4-methylmorpholin- |
| | 2-yl)methoxy)benzamide |
| 33 | 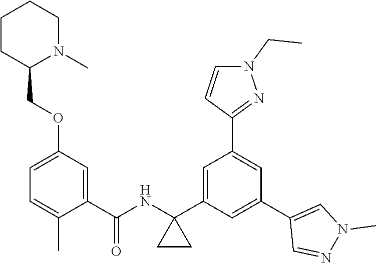 [SMILES: CCn1ccc(-c2cc(-c3cnn(C)c3)cc(C3(NC(=O)c4cc(OC[C@H]5CCCCN5C)ccc4C)CC3)c2)n1] |
| | (R)-N-(1-(3-(1-ethyl-1H-pyrazol-3-yl)-5-(1-methyl-1H-pyrazol- |
| | 4-yl)phenyl)cyclopropyl)-2-methyl- |
| | 5-((1-methylpiperidin-2-yl)methoxy)benzamide |
| 34 | 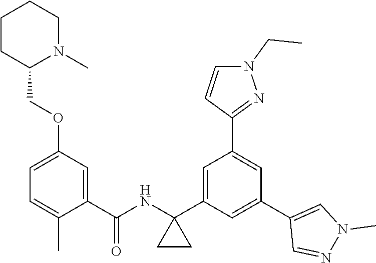 [SMILES: CCn1ccc(-c2cc(-c3cnn(C)c3)cc(C3(NC(=O)c4cc(OC[C@@H]5CCCCN5C)ccc4C)CC3)c2)n1] |
| | (S)-N-(1-(3-(1-ethyl-1H-pyrazol-3-yl)-5-(1-methyl-1H- |
| | pyrazol-4-yl)phenyl)cyclopropyl)-2-methyl- |
| | 5-((1-methylpiperidin-2-yl)methoxy)benzamide |
| 35 | 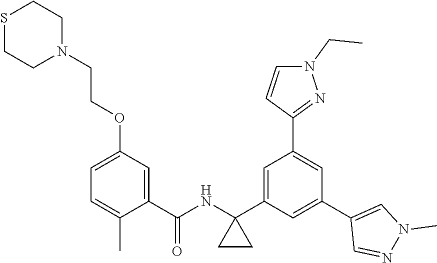 [SMILES: CCn1ccc(-c2cc(-c3cnn(C)c3)cc(C3(NC(=O)c4cc(OCCN5CCSCC5)ccc4C)CC3)c2)n1] |
| | N-(1-(3-(1-ethyl-1H-pyrazol-3-yl)-5-(1-methyl-1H-pyrazol- |
| | 4-yl)phenyl)cyclopropyl)-2-methyl-5- |
| | (2-thiomorpholinoethoxy)benzamide |
| 36 | 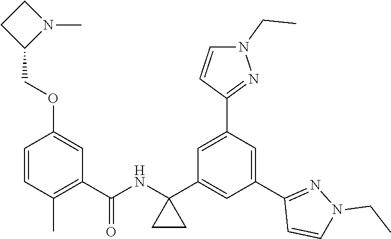 [SMILES: CCn1ccc(-c2cc(-c3ccn(CC)n3)cc(C3(NC(=O)c4cc(OC[C@@H]5CCN5C)ccc4C)CC3)c2)n1] |
| | (S)-N-(1-(3,5-bis(1-ethyl-1H-pyrazol-3-yl)phenyl)cyclopropyl)- |
| | 2-methyl-5-((1-methylazetidin-2- |
| | yl)methoxy)benzamide |
| 37 | 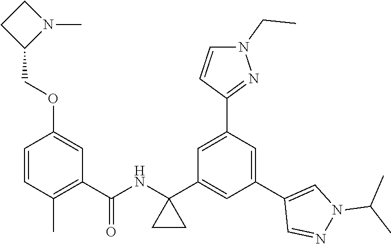 [SMILES: CCn1ccc(-c2cc(-c3cnn(C(C)C)c3)cc(C3(NC(=O)c4cc(OC[C@@H]5CCN5C)ccc4C)CC3)c2)n1] |
| | (S)-N-(1-(3-(1-ethyl-1H-pyrazol-3-yl)-5-(1-isopropyl- |
| | 1H-pyrazol-4-yl)phenyl)cyclopropyl)-2- |
| | methyl-5-((1-methylazetidin-2-yl)methoxy)benzamide |
| 38 | 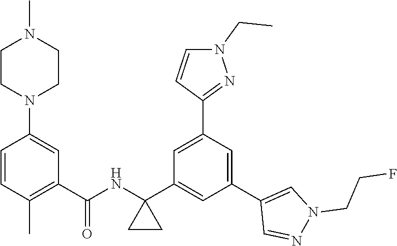 [SMILES: CCn1ccc(-c2cc(-c3cnn(CCF)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(2-fluoroethyl)- |
| | 1H-pyrazol-4-yl)phenyl)cyclopropyl)-2- |
| | methyl-5-(4-methylpiperazin-1-yl)benzamide |
| 39 | 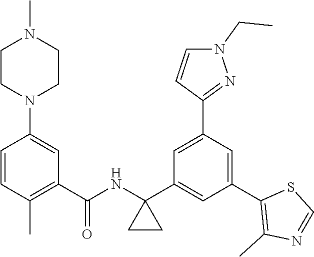 [SMILES: CCn1ccc(-c2cc(-c3scnc3C)cc(C3(NC(=O)c4cc(N5CCN(C)CC5)ccc4C)CC3)c2)n1] |
| | N-(1-(3-(1-ethyl-1H-pyrazol-3-yl)-5-(4-methylthiazol- |
| | 5-yl)phenyl)cyclopropyl)-2-methyl-5-(4- |
| | methylpiperazin-1-yl)benzamide |
| 40 | 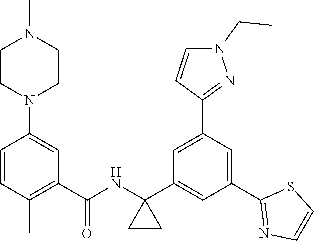 [SMILES: CCn1ccc(-c2cc(-c3nccs3)cc(C3(NC(=O)c4cc(N5CCN(C)CC5)ccc4C)CC3)c2)n1] |
| | N-(1-(3-(1-ethyl-1H-pyrazol-3-yl)-5-(thiazol-2-yl) |
| | phenyl)cyclopropyl)-2-methyl-5-(4- |
| | methylpiperazin-1-yl)benzamide |
| 41 | 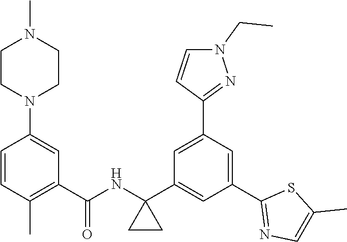 [SMILES: CCn1ccc(-c2cc(-c3ncc(C)s3)cc(C3(NC(=O)c4cc(N5CCN(C)CC5)ccc4C)CC3)c2)n1] |
| | N-(1-(3-(1-ethyl-1H-pyrazol-3-yl)-5-(5- |
| | methylthiazol-2-yl)phenyl)cyclopropyl)-2-methyl-5-(4- |
| | methylpiperazin-1-yl)benzamide |
| 42 | 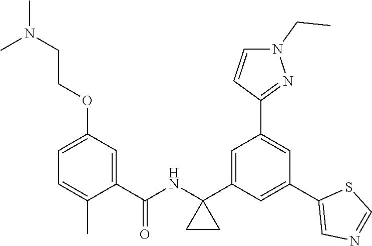 [SMILES: CCn1ccc(-c2cc(-c3cncs3)cc(C3(NC(=O)c4cc(OCCN(C)C)ccc4C)CC3)c2)n1] |
| | 5-(2-(dimethylamino)ethoxy)-N-(1-(3-(1-ethyl-1H-pyrazol-3- |
| | yl)-5-(thiazol-5-yl)phenyl)cyclopropyl)-2-methylbenzamide |
| 43 | 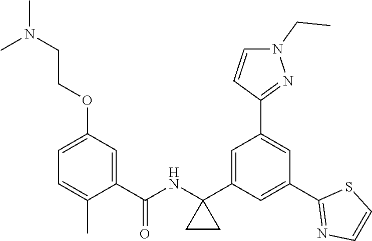 [SMILES: CCn1ccc(-c2cc(-c3nccs3)cc(C3(NC(=O)c4cc(OCCN(C)C)ccc4C)CC3)c2)n1] |
| | 5-(2-(dimethylamino)ethoxy)-N-(1-(3-(1-ethyl-1H-pyrazol- |
| | 3-yl)-5-(thiazol-2-yl)phenyl)cyclopropyl)-2-methylbenzamide |
| 44 | 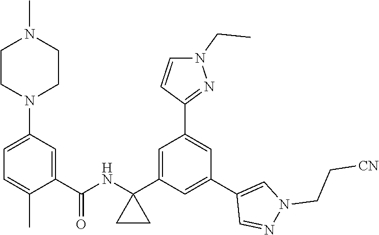 [SMILES: CCn1ccc(-c2cc(-c3cnn(CCC#N)c3)cc(C3(NC(=O)c4cc(N5CCN(C)CC5)ccc4C)CC3)c2)n1] |
| | N-(1-(3-(1-(2-cyanoethyl)-1H-pyrazol-4-yl)-5-(1-ethyl-1H- |
| | pyrazol-3-yl)phenyl)cyclopropyl)-2- |
| | methyl-5-(4-methylpiperazin-1-yl)benzamide |
| 45 | 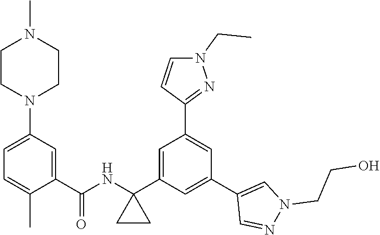 [SMILES: CCn1ccc(-c2cc(-c3cnn(CCO)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(2-hydroxyethyl)- |
| | 1H-pyrazol-4-yl)phenyl)cyclopropyl)-2- |
| | methyl-5-(4-methylpiperazin-1-yl)benzamide |
| 46 | 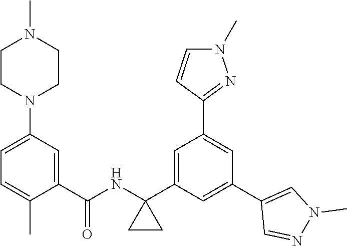 [SMILES: Cc1ccc(N2CCN(C)CC2)cc1C(=O)NC1(c2cc(-c3cnn(C)c3)cc(-c3ccn(C)n3)c2)CC1] |
| | 2-methyl-N-(1-(3-(1-methyl-1H-pyrazol-3-yl)-5-(1-methyl- |
| | 1H-pyrazol-4-yl)phenyl)cyclopropyl)-5- |
| | (4-methylpiperazin-1-yl)benzamide |
| 47 | 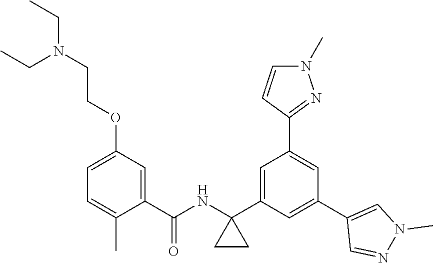 [SMILES: CCN(CC)CCOc1ccc(C)c(C(=O)NC2(c3cc(-c4cnn(C)c4)cc(-c4ccn(C)n4)c3)CC2)c1] |
| | 5-(2-(diethylamino)ethoxy)-N-(1-(3-(1-ethyl-1H-pyrazol-3- |
| | yl)-5-(1-methyl-1H-pyrazol-4- |
| | yl)phenyl)cyclopropyl)-2-methylbenzamide |
| 48 | 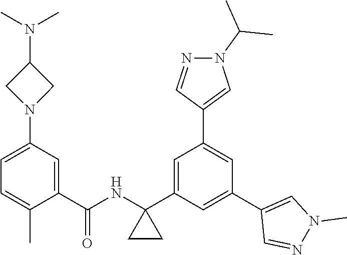 [SMILES: Cc1ccc(N2CC(N(C)C)C2)cc1C(=O)NC1(c2cc(-c3cnn(C)c3)cc(-c3cnn(C(C)C)c3)c2)CC1] |
| | 5-(3-(dimethylamino)azetidin-1-yl)-N-(1-(3-(1-isopropyl- |
| | 1H-pyrazol-4-yl)-5-(1-methyl-1H- |
| | pyrazol-4-yl)phenyl)cyclopropyl)-2-methylbenzamide |
| 49 | 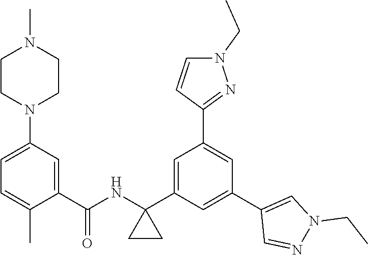 [SMILES: CCn1cc(-c2cc(-c3ccn(CC)n3)cc(C3(NC(=O)c4cc(N5CCN(C)CC5)ccc4C)CC3)c2)cn1] |
| | N-(1-(3-(1-ethyl-1H-pyrazol-3-yl)-5-(1-ethyl-1H-pyrazol- |
| | 4-yl)phenyl)cyclopropyl)-2-methyl-5-(4- |
| | methylpiperazin-1-yl)benzamide |
| 50 | 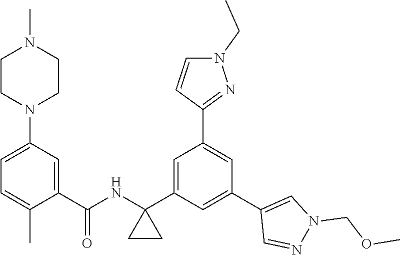 [SMILES: CCn1ccc(-c2cc(-c3cnn(COC)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(methoxymethyl)- |
| | 1H-pyrazol-4-yl)phenyl)cyclopropyl)-2- |
| | methyl-5-(4-methylpiperazin-1-yl)benzamide |
| 51 | 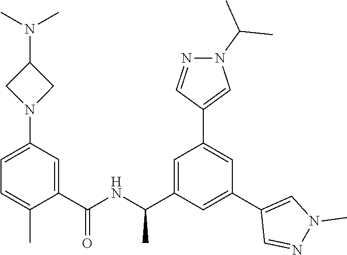 [SMILES: Cc1ccc(N2CC(N(C)C)C2)cc1C(=O)N[C@H](C)c1cc(-c2cnn(C)c2)cc(-c2cnn(C(C)C)c2)c1] |
| | (R)-5-(3-(dimethylamino)azetidin-1-yl)-N-(1-(3- |
| | (1-isopropyl-1H-pyrazol-4-yl)-5-(1-methyl-1H- |
| | pyrazol-4-yl)phenyl)ethyl)-2-methylbenzamide |
| 52 | 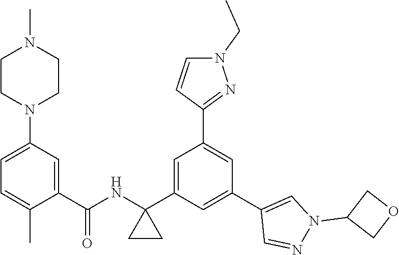 [SMILES: CCn1ccc(-c2cc(-c3cnn(C4COC4)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(oxetan-3-yl)- |
| | 1H-pyrazol-4-yl)phenyl)cyclopropyl)-2- |
| | methyl-5-(4-methylpiperazin-1-yl)benzamide |
| 53 | 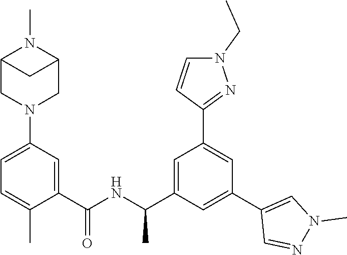 [SMILES: CCn1ccc(-c2cc(-c3cnn(C)c3)cc([C@@H](C)NC(=O)c3cc(N4CC5CC(C4)N5C)ccc3C)c2)n1] |
| | N-((R)-1-(3-(1-ethyl-1H-pyrazol-3-yl)-5-(1-methyl- |
| | 1H-pyrazol-4-yl)phenyl)ethyl)-2-methyl-5-(6- |
| | methyl-3,6-diazabicyclo[3.1.1]heptan-3-yl)benzamide |
| 54 | 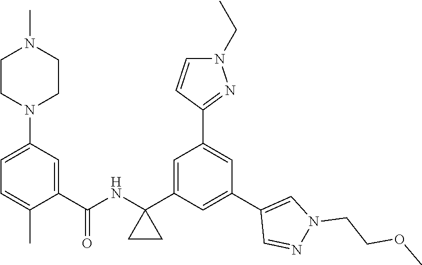 [SMILES: CCn1ccc(-c2cc(-c3cnn(CCOC)c3)cc(C3(NC(=O)c4cc(N5CCN(C)CC5)ccc4C)CC3)c2)n1] |
| | N-(1-(3-(1-ethyl-1H-pyrazol-3-yl)-5-(1-(2-methoxyethyl)- |
| | 1H-pyrazol-4-yl)phenyl)cyclopropyl)-2- |
| | methyl-5-(4-methylpiperazin-1-yl)benzamide |
| 55 | 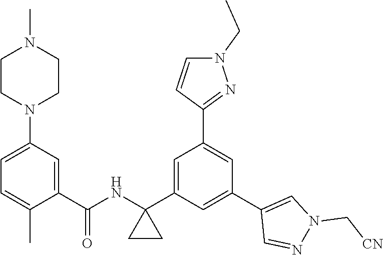 [SMILES: CCn1ccc(-c2cc(-c3cnn(CC#N)c3)cc(C3(NC(=O)c4cc(N5CCN(C)CC5)ccc4C)CC3)c2)n1] |
| | N-(1-(3-(1-(cyanomethyl)-1H-pyrazol-4-yl)-5-(1-ethyl- |
| | 1H-pyrazol-3-yl)phenyl)cyclopropyl)-2- |
| | methyl-5-(4-methylpiperazin-1-yl)benzamide |
| 56 | 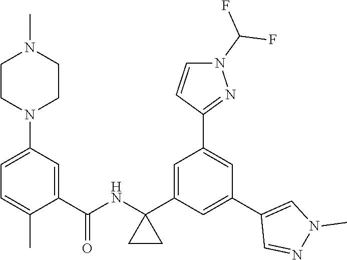 [SMILES: Cc1ccc(N2CCN(C)CC2)cc1C(=O)NC1(c2cc(-c3cnn(C)c3)cc(-c3ccn(C(F)F)n3)c2)CC1] |
| | N-(1-(3-(1-(difluoromethyl)-1H-pyrazol-3-yl)-5-(1- |
| | methyl-1H-pyrazol-4-yl)phenyl)cyclopropyl)-2- |
| | methyl-5-(4-methylpiperazin-1-yl)benzamide |
| 57 | 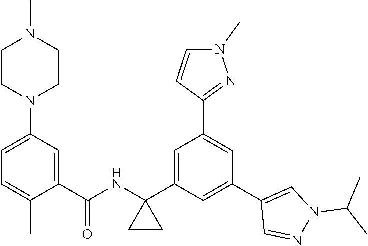 [SMILES: Cc1ccc(N2CCN(C)CC2)cc1C(=O)NC1(c2cc(-c3cnn(C(C)C)c3)cc(-c3ccn(C)n3)c2)CC1] |
| | N-(1-(3-(1-isopropyl-1H-pyrazol-4-yl)-5-(1-methyl- |
| | 1H-pyrazol-3-yl)phenyl)cyclopropyl)-2- |
| | methyl-5-(4-methylpiperazin-1-yl)benzamide |
| 58 | 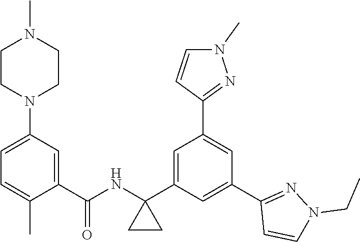 [SMILES: CCn1ccc(-c2cc(-c3ccn(C)n3)cc(C3(NC(=O)c4cc(N5CCN(C)CC5)ccc4C)CC3)c2)n1] |
| | N-(1-(3-(1-ethyl-1H-pyrazol-3-yl)-5-(1-methyl-1H- |
| | pyrazol-3-yl)phenyl)cyclopropyl)-2-methyl-5- |
| | (4-methylpiperazin-1-yl)benzamide |
| 59 | 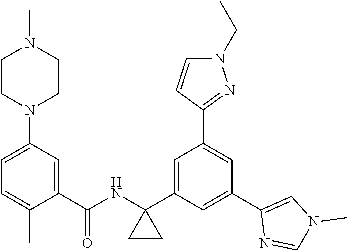 [SMILES: CCn1ccc(-c2cc(-c3cn(C)cn3)cc(C3(NC(=O)c4cc(N5CCN(C)CC5)ccc4C)CC3)c2)n1] |
| | N-(1-(3-(1-ethyl-1H-pyrazol-3-yl)-5-(1-methyl-1H- |
| | imidazol-4-yl)phenyl)cyclopropyl)-2-methyl-5- |
| | (4-methylpiperazin-1-yl)benzamide |
| 60 | 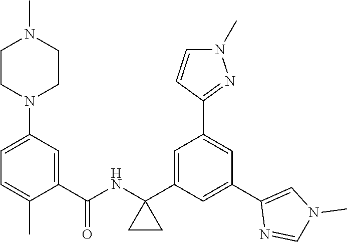 [SMILES: Cc1ccc(N2CCN(C)CC2)cc1C(=O)NC1(c2cc(-c3cn(C)cn3)cc(-c3ccn(C)n3)c2)CC1] |
| | N-(1-(3,5-bis(1-methyl-1H-pyrazol-4-yl)phenyl) |
| | cyclopropyl)-2-methyl-5-(4-methylpiperazin-1- |
| | yl)benzamide |
| 61 | 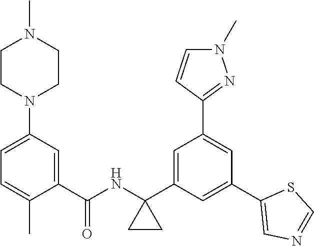 [SMILES: Cc1ccc(N2CCN(C)CC2)cc1C(=O)NC1(c2cc(-c3ccn(C)n3)cc(-c3cncs3)c2)CC1] |
| | 2-methyl-N-(1-(3-(1-methyl-1H-pyrazol-3-yl)-5- |
| | (thiazol-5-yl)phenyl)cyclopropyl)-5-(4- |
| | methylpiperazin-1-yl)benzamide |
| 62 | 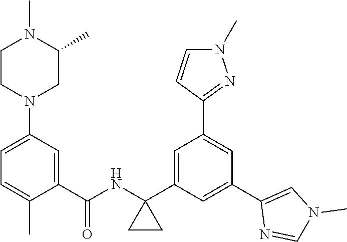 [SMILES: Cc1ccc(N2CCN(C)[C@H](C)C2)cc1C(=O)NC1(c2cc(-c3cn(C)cn3)cc(-c3ccn(C)n3)c2)CC1] |
| | (R)-5-(3,4-dimethylpiperazin-1-yl)-2-methyl-N-(1-(3- |
| | (1-methyl-1H-pyrazol-3-yl)-5-(1-methyl-1H- |
| | pyrazol-4-yl)phenyl)cyclopropyl)benzamide |
| 63 | 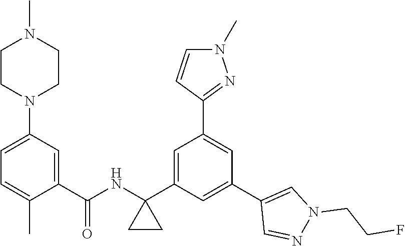 [SMILES: Cc1ccc(N2CCN(C)CC2)cc1C(=O)NC1(c2cc(-c3cnn(CCF)c3)cc(-c3ccn(C)n3)c2)CC1] |
| | N-(1-(3-(1-(2-fluoroethyl)-1H-pyrazol-4-yl)-5-(1-methyl- |
| | 1H-pyrazol-3-yl)phenyl)cyclopropyl)-2- |
| | methyl-5-(4-methylpiperazin-1-yl)benzamide |
| 64 | 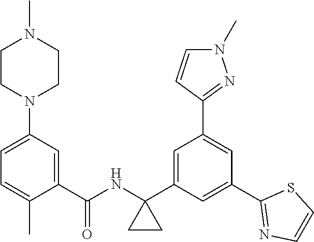 [SMILES: Cc1ccc(N2CCN(C)CC2)cc1C(=O)NC1(c2cc(-c3ccn(C)n3)cc(-c3nccs3)c2)CC1] |
| | 2-methyl-N-(1-(3-(1-methyl-1H-pyrazol-3-yl)-5- |
| | (thiazol-2-yl)phenyl)cyclopropyl)-5-(4- |
| | methylpiperazin-1-yl)benzamide |
| 65 | 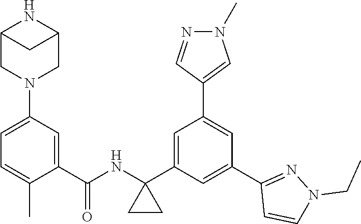 [SMILES: CCn1ccc(-c2cc(-c3cnn(C)c3)cc(C3(NC(=O)c4cc(N5CC6CC(C5)N6)ccc4C)CC3)c2)n1] |
| | 5-(3,6-diazabicyclo[3.1.1]heptan-3-yl)-N-(1-(3- |
| | (1-ethyl-1H-pyrazol-3-yl)-5-(1-methyl-1H-pyrazol- |
| | 4-yl)phenyl)cyclopropyl)-2-methylbenzamide |
| 66 | 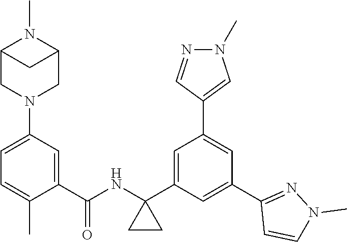 [SMILES: Cc1ccc(N2CC3CC(C2)N3C)cc1C(=O)NC1(c2cc(-c3cnn(C)c3)cc(-c3ccn(C)n3)c2)CC1] |
| | 2-methyl-N-(1-(3-(1-methyl-1H-pyrazol-3-yl)-5- |
| | (1-methyl-1H-pyrazol-4-yl)phenyl)cyclopropyl)-5- |
| | (6-methyl-3,6-diazabicyclo[3.1.1]heptan-3-yl)benzamide |
| 67 | 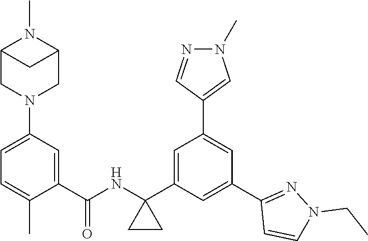 [SMILES: CCn1ccc(-c2cc(-c3cnn(C)c3)cc(C3(NC(=O)c4cc(N5CC6CC(C5)N6C)ccc4C)CC3)c2)n1] |
| | N-(1-(3-(1-ethyl-1H-pyrazol-3-yl)-5-(1-methyl-1H- |
| | pyrazol-4-yl)phenyl)cyclopropyl)-2-methyl-5- |
| | (6-methyl-3,6-diazabicyclo[3.1.1]heptan-3-yl)benzamide |
| 68 | 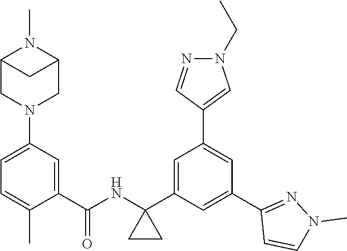 [SMILES: CCn1cc(-c2cc(-c3ccn(C)n3)cc(C3(NC(=O)c4cc(N5CC6CC(C5)N6C)ccc4C)CC3)c2)cn1] |
| | N-(1-(3-(1-ethyl-1H-pyrazol-4-yl)-5-(1-methyl-1H- |
| | pyrazol-3-yl)phenyl)cyclopropyl)-2-methyl-5- |
| | (6-methyl-3,6-diazabicyclo[3.3.1]heptan-3-yl)benzamide |
| 69 | 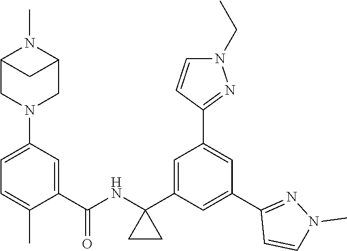 [SMILES: CCn1ccc(-c2cc(-c3ccn(C)n3)cc(C3(NC(=O)c4cc(N5CC6CC(C5)N6C)ccc4C)CC3)c2)n1] |
| | N-(1-(3-(1-ethyl-1H-pyrazol-3-yl)-5-(1-methyl-1H- |
| | pyrazol-3-yl)phenyl)cyclopropyl)-2-methyl-5- |
| | (6-methyl-3,6-diazabicyclo[3.1.1]heptan-3-yl)benzamide |
| 70 | 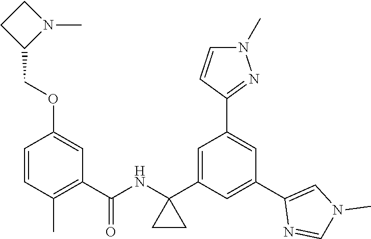 |
| | (S)-2-methyl-N-(1-(3-(1-methyl-1H-pyrazol-3-yl)-5-(1- |
| | methyl-1H-pyrazol-4-yl)phenyl)cyclopropyl)-5- |
| | ((1-methylazetidin-2-yl)methoxy)benzamide |
| 71 | 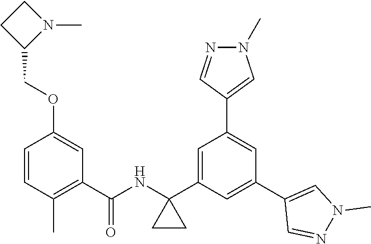 |
| | (S)-N-(1-(3,5-bis(1-methyl-1H-pyrazol-4-yl)phenyl) |
| | cyclopropyl)-2-methyl-5-((1-methylazetidin-2- |
| | yl)methoxy)benzamide |
| 72 | 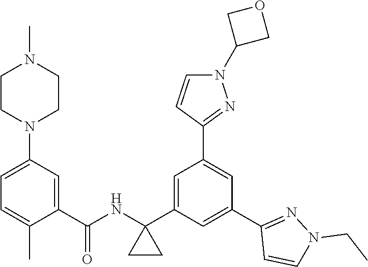 [SMILES: CCn1ccc(-c2cc(-c3ccn(C4COC4)n3)cc(C3(NC(=O)c4cc(N5CCN(C)CC5)ccc4C)CC3)c2)n1] |
| | N-(1-(3-(1-ethyl-1H-pyrazol-3-yl)-5-(1-(oxetan-3-yl)- |
| | 1H-pyrazol-3-yl)phenyl)cyclopropyl)-2- |
| | methyl-5-(4-methylpiperazin-1-yl)benzamide |
| 73 | 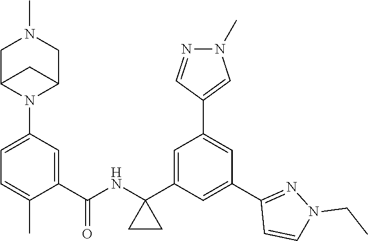 [SMILES: CCn1ccc(-c2cc(-c3cnn(C)c3)cc(C3(NC(=O)c4cc(N5C6CC5CN(C)C6)ccc4C)CC3)c2)n1] |
| | N-(1-(3-(1-ethyl-1H-pyrazol-3-yl)-5-(1-methyl-1H-pyrazol- |
| | 4-yl)phenyl)cyclopropyl)-2-methyl-5- |
| | (3-methyl-3,6-diazabicyclo[3.1.1]heptan-6-yl)benzamide |
| 74 | 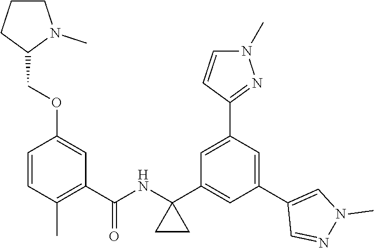 |
| | (S)-2-methyl-N-(1-(3-(1-methyl-1H-pyrazol-3-yl)-5-(1- |
| | methyl-1H-pyrazol-4-yl)phenyl)cyclopropyl)-5- |
| | ((1-methylpyrrolidin-2-yl)methoxy)benzamide |
| 75 | 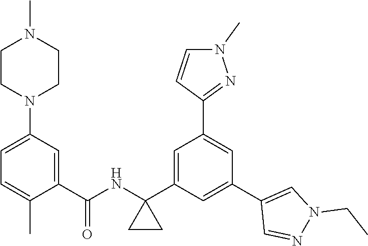 [SMILES: CCn1cc(-c2cc(-c3ccn(C)n3)cc(C3(NC(=O)c4cc(N5CCN(C)CC5)ccc4C)CC3)c2)cn1] |
| | N-(1-(3-(1-ethyl-1H-pyrazol-4-yl)-5-(1-methyl-1H- |
| | pyrazol-3-yl)phenyl)cyclopropyl)-2-methyl-5- |
| | (4-methylpiperazin-1-yl)benzamide |
| 76 | 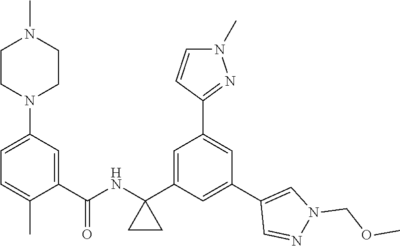 [SMILES: COCn1cc(-c2cc(-c3ccn(C)n3)cc(C3(NC(=O)c4cc(N5CCN(C)CC5)ccc4C)CC3)c2)cn1] |
| | N-(1-(3-(1-(methoxymethyl)-1H-pyrazol-4-yl)-5-(1- |
| | methyl-1H-pyrazol-3-yl)phenyl)cyclopropyl)- |
| | 2-methyl-5-(4-methylpiperazin-1-yl)benzamide |
| 77 | 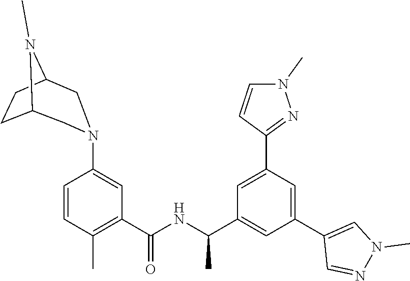 [SMILES: Cc1ccc(N2CC3CCC(C2)N3C)cc1C(=O)N[C@H](C)c1cc(-c2cnn(C)c2)cc(-c2ccn(C)n2)c1] |
| | 2-methyl-N-((R)-1-(3-(1-methyl-1H-pyrazol-3-yl)-5-(1- |
| | methyl-1H-pyrazol-4-yl)phenyl)ethyl)-5- |
| | ((1R,5S)-8-methyl-3,8-diazabicyclo[3.2.1]octan-3-yl)benzamide |
| 78 | 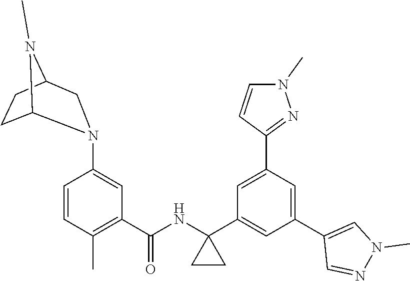 [SMILES: Cc1ccc(N2CC3CCC(C2)N3C)cc1C(=O)NC1(c2cc(-c3cnn(C)c3)cc(-c3ccn(C)n3)c2)CC1] |
| | 2-methyl-N-(1-(3-(1-methyl-1H-pyrazol-3-yl)-5- |
| | (1-methyl-1H-pyrazol-4-yl)phenyl)cyclopropyl)-5- |
| | ((1R,5S)-8-methyl-3,8-diazbicyclo[3.2.1]octan-3-yl)benzamide |
| 79 | 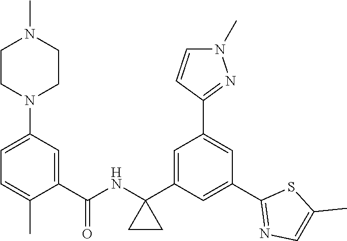 [SMILES: Cc1cnc(-c2cc(-c3ccn(C)n3)cc(C3(NC(=O)c4cc(N5CCN(C)CC5)ccc4C)CC3)c2)s1] |
| | 2-methyl-N-(1-(3-(1-methyl-1H-pyrazol-3-yl)-5- |
| | (5-methylthiazol-2-yl)phenyl)cyclopropyl)-5-(4- |
| | methylpiperazin-1-yl)benzamide |
| 80 | 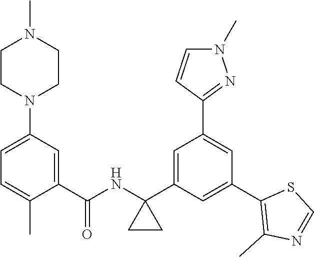 [SMILES: Cc1ccc(N2CCN(C)CC2)cc1C(=O)NC1(c2cc(-c3ccn(C)n3)cc(-c3scnc3C)c2)CC1] |
| | 2-methyl-N-(1-(3-(1-methyl-1H-pyrazol-3-yl)-5- |
| | (4-methylthiazol-5-yl)phenyl)cyclopropyl)-5-(4- |
| | methylpiperazin-1-yl)benzamide |
| 81 | 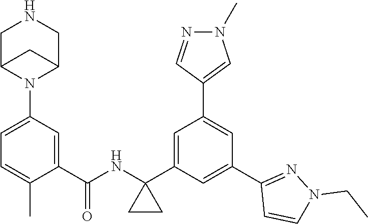 [SMILES: CCn1ccc(-c2cc(-c3cnn(C)c3)cc(C3(NC(=O)c4cc(N5C6CNCC5C6)ccc4C)CC3)c2)n1] |
| | 5-(3,6-diazabicyclo[3.1.1]heptan-6-yl)-N-(1-(3-(1- |
| | ethyl-1H-pyrazol-3-yl)-5-(1-methyl-1H-pyrazol- |
| | 4-yl)phenyl)cyclopropyl)-2-methylbenzamide |
| 82 | 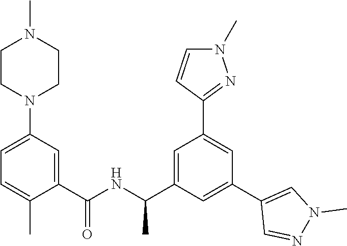 [SMILES: Cc1ccc(N2CCN(C)CC2)cc1C(=O)N[C@H](C)c1cc(-c2cnn(C)c2)cc(-c2ccn(C)n2)c1] |
| | (R)-2-methyl-N-(1-(3-(1-methyl-1H-pyrazol-3-yl)-5- |
| | (1-methyl-1H-pyrazol-4-yl)phenyl)ethyl)-5-(4- |
| | methylpiperazin-1-yl)benzamide |
| 83 | 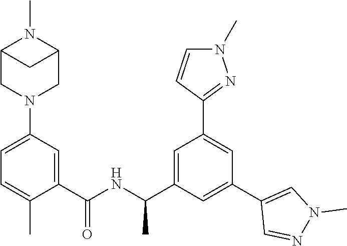 [SMILES: Cc1ccc(N2CC3CC(C2)N3C)cc1C(=O)N[C@H](C)c1cc(-c2cnn(C)c2)cc(-c2ccn(C)n2)c1] |
| | 2-methyl-N-((R)-1-(3-(1-methyl-1H-pyrazol-3-yl)-5- |
| | (1-methyl-1H-pyrazol-4-yl)phenyl)ethyl)-5-(6- |
| | methyl-3,6-diazabicyclo[3.1.1]heptan-3-yl)benzamide |
| 84 | 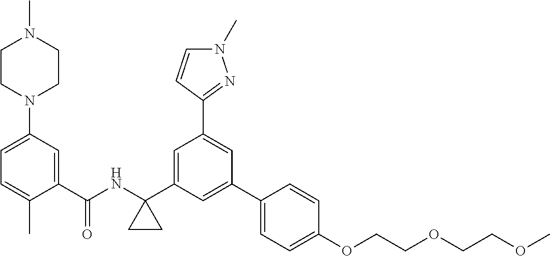 [SMILES: COCCOCCOc1ccc(-c2cc(-c3ccn(C)n3)cc(C3(NC(=O)c4cc(N5CCN(C)CC5)ccc4C)CC3)c2)cc1] |
| | N-(1-(4′-(2-(2-methoxyethoxy)ethoxy)-5-(1-methyl-1H-pyrazol-3-yl)-[1,1′-biphenyl]-3- |
| | yl)cyclopropyl)-2-methyl-5-(4-methylpiperazin-1-yl)benzamide |
| 85 | 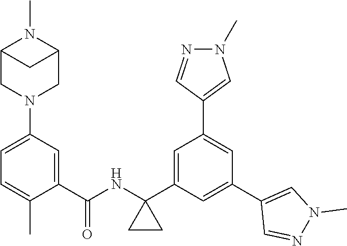 [SMILES: Cc1ccc(N2CC3CC(C2)N3C)cc1C(=O)NC1(c2cc(-c3cnn(C)c3)cc(-c3cnn(C)c3)c2)CC1] |
| | N-(1-(3,5-bis(1-methyl-1H-pyrazol-4-yl)phenyl)cyclopropyl)-2-methyl-5- |
| | (6-methyl-3,6-diazabicyclo[3.1.1]heptan-3-yl)benzamide |
| 86 | 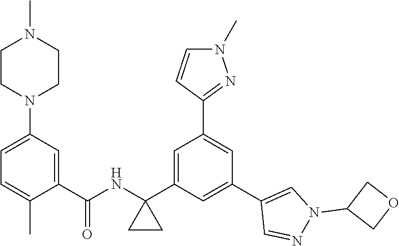 [SMILES: Cc1ccc(N2CCN(C)CC2)cc1C(=O)NC1(c2cc(-c3cnn(C4COC4)c3)cc(-c3ccn(C)n3)c2)CC1] |
| | 2-methyl-N-(1-(3-(1-methyl-1H-pyrazol-3-yl)-5-(1-(oxetan-3-yl)-1H-pyrazol-4- |
| | yl)phenyl)cyclopropyl)-5-(4-methylpiperazin-1-yl)benzamide |
| 87 | 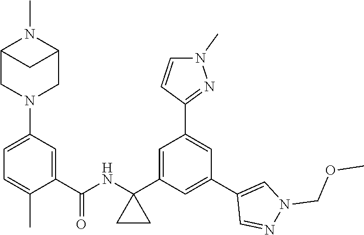 [SMILES: COCn1cc(-c2cc(-c3ccn(C)n3)cc(C3(NC(=O)c4cc(N5CC6CC(C5)N6C)ccc4C)CC3)c2)cn1] |
| | N-(1-(3-(1-ethyl-1H-pyrazol-3-yl)-5-(1-(methoxymethyl)- |
| | 1H-pyrazol-4-yl)phenyl)cyclopropyl)-2- |
| | methyl-5-(6-methyl-3,6-diazabicyclo[3.1.1]heptan-3-yl)benzamide |
| 88 | 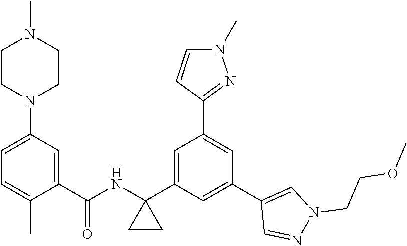 [SMILES: COCCn1cc(-c2cc(-c3ccn(C)n3)cc(C3(NC(=O)c4cc(N5CCN(C)CC5)ccc4C)CC3)c2)cn1] |
| | N-(1-(3-(1-(2-methoxyethyl)-1H-pyrazol-4-yl)-5-(1-methyl-1H-pyrazol- |
| | 3-yl)phenyl)cyclopropyl)-2-methyl-5-(4-methylpiperazin-1-yl)benzamide |

In another aspect, the disclosure provides a pharmaceutical composition comprising at least one compound of the disclosure and at least one pharmaceutically acceptable carrier.

In certain embodiments, the pharmaceutical composition further comprises at least one additional therapeutically effective agent. In certain embodiments, the pharmaceutical composition further comprise at least one antiviral agent. In certain embodiments, the antiviral agent is a protease inhibitor. In certain embodiments, the antiviral agent is selected from the group consisting of nirmatrelvir, ensiltrelvir, molnupiravir, VV116, and remdesivir.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods

In another aspect, the disclosure provides a method for promoting an antiviral effect in a subject. In certain embodiments, the method comprises administering at least one compound of the disclosure and/or the pharmaceutical composition of the disclosure. In certain embodiments, the method further comprises administering to the subject at least one antiviral agent. In certain embodiments, the antiviral agent is a protease inhibitor. In certain embodiments, the antiviral agent is selected from the group consisting of nirmatrelvir, ensiltrelvir, molnupiravir, VV116, and remdesivir.

In another aspect, the disclosure provides a method for inhibiting a papain-like protease in a subject. In certain embodiments, the method further comprises administering to the subject at least one compound of the disclosure and/or the pharmaceutical composition of the disclosure.

In another aspect, the disclosure provides a method for treating, preventing, and/or ameliorating a viral infection in a subject. In certain embodiments, the method comprises administering at least one compound of the disclosure and/or the pharmaceutical composition of the disclosure. In certain embodiments, the viral infection is caused by a coronavirus. In certain embodiments, the coronavirus is SARS-CoV-2. In certain embodiments, the viral infection is COVID-19.

In certain embodiments, the subject is a mammal. In certain embodiments, the mammal is a human.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time.

Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### Examples

Various embodiments of the present application can be better understood by reference to the following Examples which are offered by way of illustration. The scope of the present application is not limited to the Examples given herein.

Compound Synthesis The disclosure provides non-limiting, exemplary synthetic procedures for certain compounds of the disclosure. In certain embodiments, compounds of the disclosure are prepared according to Method A (Schemes 1-2). In other embodiments, compounds of the disclosure are prepared according to Method B (Schemes 3-5).

### Method A

In certain embodiments, a compound of Formula (I) wherein L¹ is

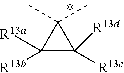

### and R¹ is

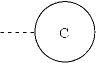

can be prepared according to Scheme 1 (e.g., wherein the R^{X} and R^{Y} in N(R^{X})(R^{Y}) can combine to form ring C).

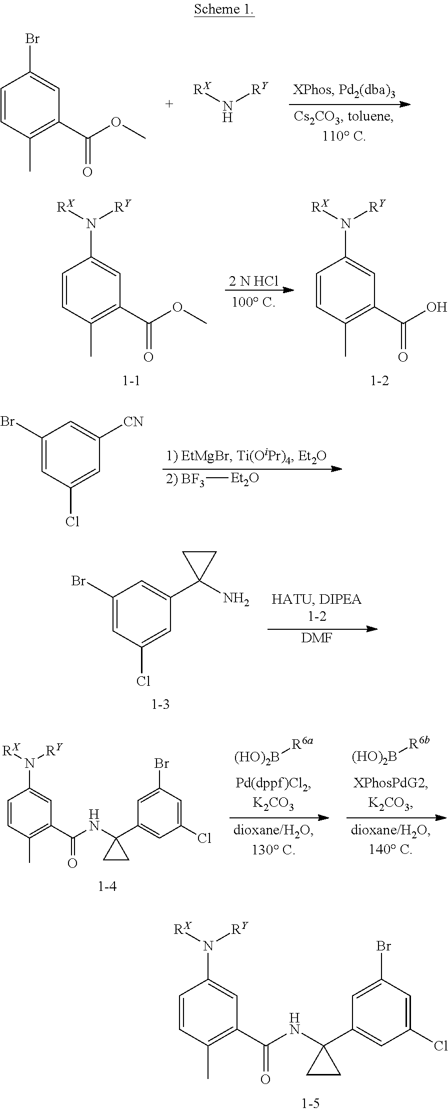

In certain embodiments, a compound of Formula (I) wherein L¹ is

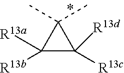

### and R¹ is

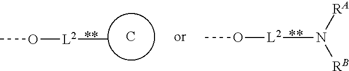

can be prepared according to Scheme 2 (e.g., wherein R^{Z} is L²-**N(R^{A})(R^{B}) or L²-[ring C]).

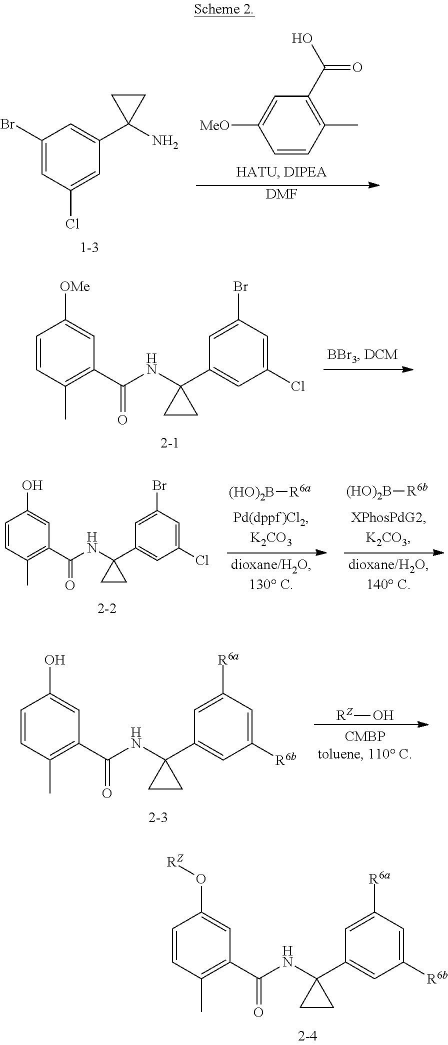

General procedures for the Buchwald coupling. To a solution of methyl 5-bromo-2-methylbenzoate (1.0 equiv) in dry toluene in sealed tube, corresponding amine (1.5 equiv), XPhos (0.04 equiv), Pd₂(dba)₃ (0.02 equiv) and Cs₂CO₃ (2.0 equiv) were added. The reaction mixture was degassed and purged with argon, and then the tube was sealed and heated to 110° C. for overnight. The mixture was diluted with EtOAc and then washed with water and brine. The organic layer was dried over Na₂SO₄, filtered, and concentrated under vacuum to give a residue which was purified by flash silica gel column chromatography (DCM/MeOH=20:1) to provide the desired product 1-1.

General procedures for the hydrolysis. A solution of compound 1-1 (1.0 equiv) in HCl (2N aqueous) was stirred at 110° C. for overnight. When the starting material was consumed entirely as monitored by LCMS, the mixture was allowed to cool to room temperature and concentrated under vacuum to remove the water completely to provide the desired product 1-2.

1-(3-bromo-5-chlorophenyl)cyclopropan-1-amine (1-3). To a solution of 3-bromo-5-chlorobenzonitrile (1.0 equiv) in dry Et₂O (0.1 M) under an Ar atmosphere was added Ti(OⁱPr)₄ (1.1 equiv) slowly and then EtMgBr (3M in Et₂O, 2.2 equiv) was added dropwise to maintain the temperature around −78° C. over 10 minutes. After the addition was complete, the mixture was stirred at −78° C. for 10 minutes and warmed to 20° C. over 1 hour. And then BF₃-Et₂O (2.0 equiv) was added slowly, the mixture was stirred at room temperature for another 1 hour. LCMS indicated that the starting material was completely consumed, the mixture was poured into 1M HCl solution and extracted with EtOAc. The aqueous phase was basified to pH 10 using 2M NaOH and extracted with DCM for several times, the combined organic layers was dried over Na₂SO₄, filtered, and concentrated under vacuum to give a residue which was purified by flash silica gel column chromatography (Hexanes/EtOAc=1:1) to get the desired product 1-3.

General amide coupling procedure. Corresponding acid (1.1 equiv), HATU (1.1 equiv) and DIPEA (3.0 equiv) were dissolved in dry DMF (0.2 M) and stirred for 15 min. After that compound 1-3 was added and the solution was stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue that was purified by silica gel column chromatography (DCM/MeOH=20:1) to provide the corresponding intermediate 1-4.

General procedures for the Suzuki coupling. To a solution of compound 1-4 or 2-2 (1.0 equiv) in dioxane/H₂O (4:1) in a microwave reaction vial was added corresponding boronic acid or ester (1.2 equiv) and potassium carbonate (1.8 equiv). The mixture was purged with nitrogen for 5 min. Then Pd(dppf)Cl₂ (0.1 equiv) was added and the solution was heated in the biotage microwave reactor at 130° C. for 90 min. The reaction mixture was diluted with EtOAc and extracted with aqueous NaHCO₃ solution and brine. The organic layer was separated, dried over anhydrous Na₂SO₄, filtered, and concentrated under vacuum to get intermediate. To a solution of intermediate (1.0 equiv) in dioxane/H₂O (4:1) in a microwave reaction vial was added corresponding boronic acid or ester (1.2 equiv) and tripotassium phosphate (1.8 equiv). The mixture was purged with nitrogen for 5 min. Then XPhosPdG2 (0.1 equiv) was added and the solution was heated in the biotage microwave reactor at 140° C. for 90 min. After the reaction completed, the reaction mixture was diluted with EtOAc and washed with water and brine. The organic layer was separated, dried over anhydrous Na₂SO₄, filtered, and concentrated under vacuum to give a residue which was purified by Prep-HPLC to give the final product.

N-(1-(3-bromo-5-chlorophenyl)cyclopropyl)-5-methoxy-2-methylbenzamide (2-1). 5-methoxy-2-methylbenzoic acid (1.1 equiv), HATU (1.1 equiv) and DIPEA (3.0 equiv) were dissolved in dry DMF (0.2 M) and stirred for 15 min. After that compound 1-3 was added and the solution was stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue that was purified by silica gel column chromatography (Hexanes/EtOAc=2:1) to provide the desired product 2-1.

N-(1-(3-bromo-5-chlorophenyl)cyclopropyl)-5-hydroxy-2-methylbenzamide. To a solution of compound 2-1 (1.0 equiv) in dry DCM at 0° C. and BBr₃ (1 M solution in DCM, 3.0 equiv) was added slowly and stirred for 3 h. After the reaction completed, the reaction was quenched with saturated NaHCO₃ solution and was then washed with DCM, water, and brine, respectively. The organic layer was dried over Na₂SO₄, filtered, and concentrated under vacuum to give a residue which was purified by flash silica gel column chromatography (Hexanes/EtOAc=1:1) to provide the desired product 2-2.

General procedures for the Mitsunobu reaction. To a solution of compound 2-3 (1.0 equiv) in dry toluene in a 15 mL sealed tube, corresponding alcohol or thiol (1.5 equiv) and cyanomethylene trimethylphosphorane (CMBP) (2.0 equiv) were added. The reaction mixture was degassed and purged with argon, and then the tube was sealed and heated to 110° C. for overnight. When the starting material was consumed entirely as monitored by LCMS, the mixture was diluted with EtOAc and washed with water and brine. The organic layer was dried over Na₂SO₄, filtered, and concentrated under vacuum to give a residue which was purified by Prep-HPLC to give the final product.

### Method B

In certain embodiments, a compound of Formula (I) wherein L¹ is

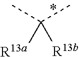

### and R¹ is

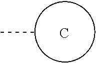

can be prepared according to Schemes 3-4 (e.g., wherein the R^{X} and R^{Y} in N(R^{X})(R^{Y}) can combine to form ring C).

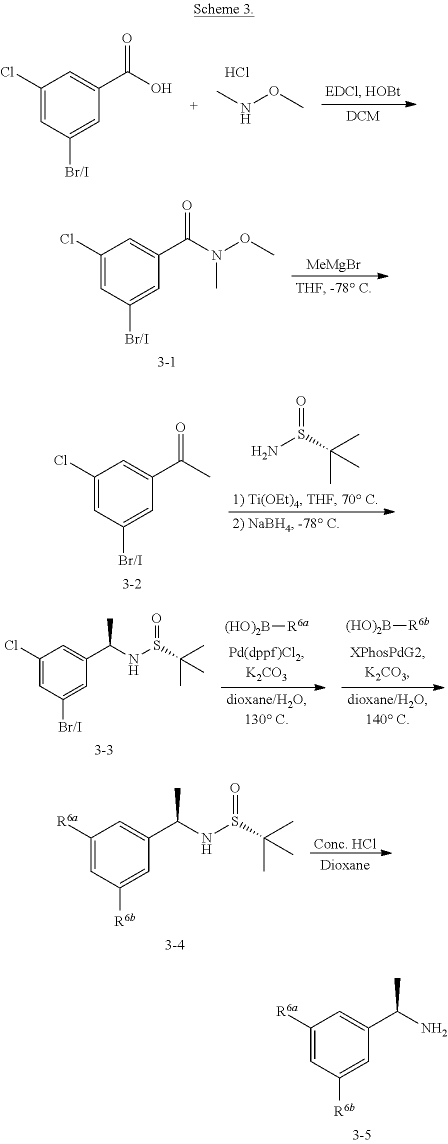

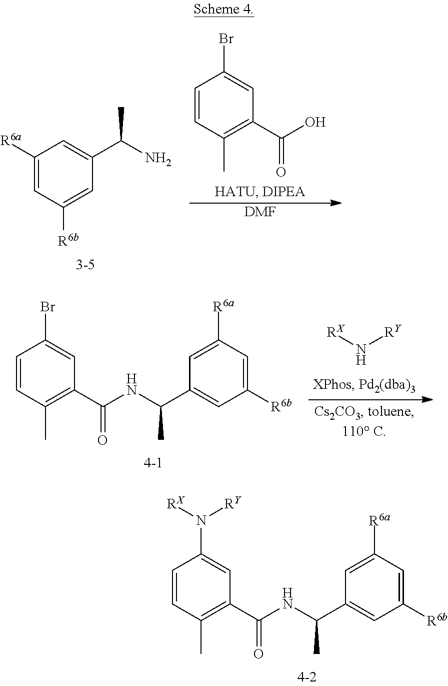

In certain embodiments, a compound of Formula (I) wherein L¹ is

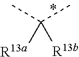

### and R¹ is

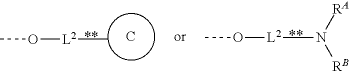

can be prepared according to Schemes 3 and 5 (e.g., wherein R^{Z} is L²-**N(R^{A})(R^{B}) or L²-[ring C]).

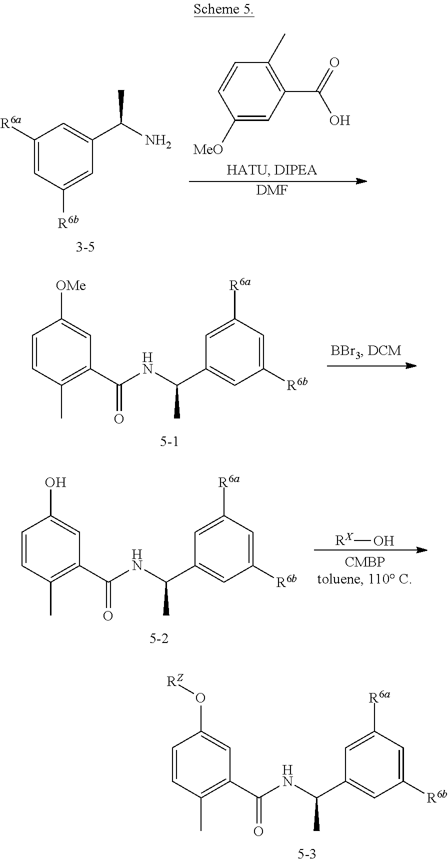

General procedures for ketone formation. To a solution of corresponding acid (1.0 equiv), N,O-dimethylhydroxylamine hydrochloride (1.2 equiv), HOBt (1.3 equiv) and EDCI (1.3 equiv) in DCM (0.1 M) was dropped triethylamine (3 equiv) at 0° C. The mixture was stirred at room temperature for 48 hours. After the reaction completed, the organic layer was washed with water and brine, dried over Na₂SO₄, filtered, and concentrated under vacuum to give product 3-1 for next step without purification. To a solution of compound 3-1 (1.0 equiv) in dry ether at −78° C. under N₂. The methylmagnesium bromide (3 M solution in diethyl ether, 3.0 equiv) was added slowly. After warming to room temperature and being stirred for 3 hours, the reaction mixture was quenched with ice cold saturated NH₄Cl and the washed with water and brine, dried over Na₂SO₄, filtered, and concentrated under vacuum to give a residue which was purified by silica gel column chromatography (Hexanes/EtOAc=10:1) to provide the desired product 3-2.

General procedures for reduction elimination. To a solution of Ti(OEt)₄ (1.44 equiv) and compound 3-2 (1.0 equiv) in dry THF (0.1 M) under an Ar atmosphere was added (R)-2-methylpropane-2-sulfinamide (1.5 equiv) and the mixture was heated to 70° C. for overnight. TLC indicated that the starting material was completely consumed. The mixture was cooled to −78° C. and NaBH₄ (3.0 equiv) was added slowly. The mixture was stirred at −78° C. for 3 h, and then warmed up to room temperature for another 3 h, MeOH was added dropwise until gas was no longer evolved. The resulting suspension was filtered through a plug of Celite and the filter cake was washed with EtOAc. The filtrate was washed with brine, and the brine layer was extracted with EtOAc. The combined organic phase was dried over Na₂SO₄, filtered, and concentrated under vacuum to give a residue which was purified by flash silica gel column chromatography (Hexanes/EtOAc=3:1) to get the desired product 3-3.

General procedures for the Suzuki coupling. To a solution of compound 3-3 (1.0 equiv) in dioxane/H₂O (4:1) in a microwave reaction vial was added corresponding boronic acid or ester (1.2 equiv) and potassium carbonate (1.8 equiv). The mixture was purged with nitrogen for 5 min. Then Pd(dppf)Cl₂ (0.1 equiv) was added and the solution was heated in the biotage microwave reactor at 130° C. for 90 min. The reaction mixture was diluted with EtOAc and extracted with aqueous NaHCO₃ solution and brine. The organic layer was separated, dried over anhydrous Na₂SO₄, filtered, and concentrated under vacuum to get intermediate. To a solution of intermediate (1 equiv) in dioxane/H₂O (4:1) in a microwave reaction vial was added corresponding boronic acid or ester (1.2 equiv) and tripotassium phosphate (1.8 equiv). The mixture was purged with nitrogen for 5 min. Then XPhosPdG2 (0.1 equiv) was added and the solution was heated in the biotage microwave reactor at 140° C. for 90 min. After the reaction completed, the reaction mixture was diluted with EtOAc and washed with water and brine. The organic layer was separated, dried over anhydrous Na₂SO₄, filtered, and concentrated under vacuum to give a residue which was purified by flash silica gel column chromatography (DCM/MeOH=20:1) to get the desired product 3-4.

General procedures for deprotection. To a solution of compound 3-4 (1.0 equiv) in 1,4-dioxane (0.1 M), concentrated HCl was added. The reactions were stirred at room temperature for 10 min. LCMS indicated that the starting material was completely consumed, the mixture was concentrated under vacuum to get general intermediate 3-5 as white solid for next step without purification.

General amide coupling procedure. Corresponding acid (1.1 equiv), HATU (1.1 equiv) and DIPEA (3.0 equiv) were dissolved in dry DMF (0.2 M) and stirred for 15 min. After that compound 3-4 was added and the solution was stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue that was purified by silica gel column chromatography (DCM/MeOH=20:1) to provide the corresponding intermediates (e.g., 4-1 and 5-1).

General procedures for the Buchwald coupling. To a solution of compound 4-1 (1.0 equiv) in dry toluene (8 mL) in sealed tube, corresponding amine (1.5 equiv), XPhos (0.04 equiv), Pd₂(dba)₃ (0.02 equiv) and Cs₂CO₃ (2.0 equiv) were added. The reaction mixture was degassed and purged with argon, and then the tube was sealed and heated to 110° C. overnight.

The mixture was diluted with EtOAc and then washed with water and brine. The organic layer was dried over Na₂SO₄, filtered, and concentrated under vacuum to give a residue which was purified by Prep-HPLC to give the final product.

General procedures for demethylation reaction. To a solution of compound 5-1 (1.0 equiv) in dry DCM at 0° C. and BBr₃ (1 M solution in DCM, 3.0 equiv) was added slowly and stirred for 3 h. After the reaction completed, the reaction was quenched with saturated NaHCO₃ solution and was then washed with DCM, water, and brine, respectively. The organic layer was dried over Na₂SO₄, filtered, and concentrated under vacuum to give a residue which was purified by flash silica gel column chromatography (DCM/MeOH=10:1) to provide the desired product 5-2.

General procedures for the Mitsunobu reaction. To a solution of compound 5-2 (1.0 equiv) in dry toluene in a 15 mL sealed tube, corresponding alcohol or thiol (1.1 equiv) and cyanomethylene trimethylphosphorane (CMBP) (1.5 equiv) were added. The reaction mixture was degassed and purged with argon, and then the tube was sealed and heated to 110° C. for overnight. When the starting material was consumed entirely as monitored by LCMS, the mixture was diluted with EtOAc and washed with water and brine. The organic layer was dried over Na₂SO₄, filtered, and concentrated under vacuum to give a residue which was purified by Prep-HPLC to give the final product.

### Example 1: (S)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide

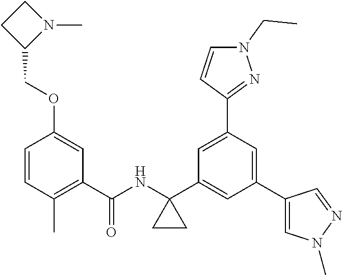

(Method A) White solid, 81% yield. ¹H NMR (400 MHz, DMSO-d₆) δ 9.03 (s, 1H), 8.20 (s, 1H), 7.88 (s, 1H), 7.79 (d, J=2.2 Hz, 1H), 7.75 (s, 1H), 7.64 (s, 1H), 7.27-7.19 (m, 2H), 6.99 (q, J=10.0, 7.0 Hz, 2H), 6.72 (d, J=2.2 Hz, 1H), 5.19 (s, 1H), 4.69 (s, 1H), 4.33 (d, J=5.4 Hz, 1H), 4.18 (q, J=7.3 Hz, 2H), 4.08 (dd, J=10.1, 5.3 Hz, 1H), 3.88 (s, 3H), 3.71 (s, 1H), 3.25 (dd, J=48.9, 36.5 Hz, 1H), 2.97-2.87 (m, 3H), 2.42 (dd, J=11.1, 7.0 Hz, 1H), 2.32 (s, 3H), 1.42 (t, J=7.2 Hz, 3H), 1.39-1.34 (m, 2H), 1.30 (d, J=4.6 Hz, 2H). ¹³C NMR (101 MHz, DMSO-d₆) δ 169.65, 169.60, 158.84, 158.51, 155.78, 154.38, 150.38, 144.73, 138.77, 138.70, 136.48, 134.30, 133.20, 132.02, 131.12, 128.45, 122.53, 120.49, 120.24, 119.82, 115.85, 115.56, 115.28, 114.16, 102.89, 76.01, 68.26, 67.08, 60.23, 53.08, 46.81, 41.47, 39.10, 34.86, 34.84, 18.91, 18.89, 18.35, 15.99. C₃₁H₃₆N₆O₂, HRMS calculated for m/z [M+H]⁺. 525.2978 (calculated), 525.2985 (found).

### Example 2: (R)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide

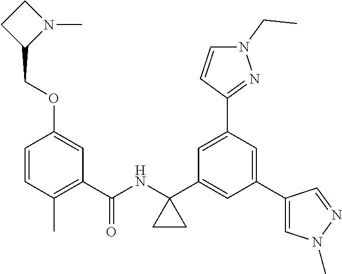

(Method A) White solid, 83% yield. ¹H NMR (400 MHz, DMSO-d₆) δ 8.97 (s, 1H), 8.13 (s, 1H), 7.81 (s, 1H), 7.72 (s, 1H), 7.68 (s, 1H), 7.58 (s, 1H), 7.15 (dd, J=13.1, 8.7 Hz, 2H), 6.95-6.85 (m, 2H), 6.65 (s, 1H), 4.61 (q, J=7.6 Hz, 1H), 4.26 (d, J=5.4 Hz, 1H), 4.10 (q, J=7.3 Hz, 2H), 4.00 (dt, J=9.8, 5.2 Hz, 1H), 3.81 (s, 3H), 3.65 (d, J=13.2 Hz, 1H), 3.34-2.98 (m, 1H), 2.82 (d, J=4.1 Hz, 3H), 2.60 (dd, J=84.9, 7.4 Hz, 1H), 2.33 (q, J=9.4 Hz, 1H), 2.24 (s, 3H), 1.34 (t, J=7.2 Hz, 3H), 1.32-1.27 (m, 2H), 1.23 (d, J=5.0 Hz, 2H). ¹³C NMR (101 MHz, DMSO-d₆) δ 169.67, 159.01, 158.67, 155.78, 154.38, 150.37, 144.75, 138.67, 136.48, 134.29, 133.21, 132.01, 131.13, 128.46, 122.53, 120.44, 120.22, 119.79, 115.82, 115.29, 114.16, 102.89, 68.22, 67.08, 53.04, 46.81, 41.43, 39.10, 34.85, 34.82, 18.92, 18.37, 15.99. C₃₁H₃₆N₆O₂, HRMS calculated for m/z [M+H]⁺: 525.2978 (calculated), 525.2984 (found).

### Example 3: (S)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide

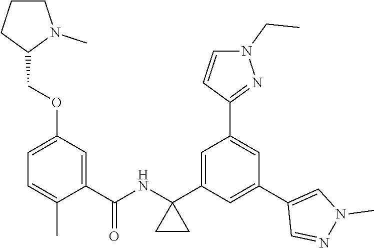

(Method A) White solid, 85% yield. ¹H NMR (400 MHz, DMSO-d₆) δ 9.04 (s, 1H), 8.20 (s, 1H), 7.89 (s, 1H), 7.79 (s, 1H), 7.76 (s, 1H), 7.65 (s, 1H), 7.25 (d, J=2.0 Hz, 1H), 7.23-7.19 (m, 1H), 7.04-6.98 (m, 2H), 6.72 (s, 1H), 4.35 (dd, J=11.1, 3.5 Hz, 1H), 4.24 (dd, J=11.0, 7.3 Hz, 1H), 4.18 (q, J=7.3 Hz, 2H), 3.88 (s, 3H), 3.85-3.76 (m, 1H), 3.61 (td, J=12.2, 4.1 Hz, 1H), 3.48-3.27 (m, 1H), 3.15 (dt, J=15.5, 8.1 Hz, 1H), 2.98 (d, J=4.1 Hz, 2H), 2.79 (d, J=4.5 Hz, 1H), 2.32 (s, 3H), 2.27 (dd, J=13.6, 6.9 Hz, 1H), 2.12-2.02 (m, 1H), 1.94 (q, J=7.7, 7.2 Hz, 1H), 1.91-1.80 (m, 1H), 1.42 (t, J=7.2 Hz, 3H), 1.39-1.34 (m, 2H), 1.29 (d, J=6.5 Hz, 2H). ¹³C NMR (101 MHz, DMSO-d₆) δ 169.67, 155.77, 150.38, 144.75, 138.67, 136.48, 134.30, 133.21, 132.01, 131.12, 128.46, 122.53, 120.46, 120.22, 119.80, 114.09, 102.88, 67.22, 66.72, 57.07, 46.81, 41.13, 39.10, 34.85, 26.77, 22.49, 18.92, 18.37, 15.99. C₃₂H₃₈N₆O₂, HRMS calculated for m/z [M+H]⁺: 539.3134 (calculated), 539.3140 (found)

### Example 4: 5-(3-(dimethylamino)azetidin-1-yl)-N-(1-(3-(1-ethyl-1H-pyrazol-3-yl)-5-(1-methyl-1H-pyrazol-4-yl)phenyl)cyclopropyl)-2-methylbenzamide

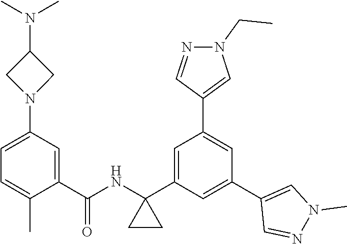 [SMILES: CCn1cc(-c2cc(-c3cnn(C)c3)cc(C3(NC(=O)c4cc(N5CC(N(C)C)C5)ccc4C)CC3)c2)cn1]

(Method A) White solid, 79% yield. ¹H NMR (400 MHz, DMSO-d₆) δ 8.90 (s, 1H), 8.12 (s, 1H), 7.80 (s, 1H), 7.72 (s, 1H), 7.68 (s, 1H), 7.55 (s, 1H), 7.18 (s, 1H), 7.06-7.00 (m, 1H), 6.64 (s, 1H), 6.43 (s, 2H), 4.16 (d, J=6.6 Hz, 1H), 4.10 (q, J=7.3 Hz, 2H), 4.03 (t, J=8.0 Hz, 2H), 3.86 (dd, J=8.8, 5.2 Hz, 2H), 3.81 (s, 3H), 2.74 (s, 6H), 2.19 (s, 3H), 1.35 (t, J=7.2 Hz, 3H), 1.30 (d, J=7.5 Hz, 2H), 1.22-1.18 (m, 2H). ¹³C NMR (101 MHz, DMSO-d₆) δ 170.17, 150.38, 148.99, 144.84, 138.11, 136.46, 134.30, 133.15, 131.46, 131.11, 128.44, 124.93, 122.54, 120.49, 120.15, 119.76, 113.32, 111.08, 102.89, 55.72, 54.45, 46.81, 40.39, 39.11, 34.79, 18.92, 18.42, 16.00. C₃₁H₃₇N₇O, MS calculated for m/z [M+H]⁺. 524.3138 (calculated), 524.3145 (found).

### Example 5: N-(1-(3-(1-ethyl-1H-pyrazol-3-yl)-5-(1-methyl-1H-pyrazol-4-yl)phenyl)cyclopropyl)-5-(((2S,4S)-4-fluoro-1-methylpyrrolidin-2-yl)methoxy)-2-methylbenzamide

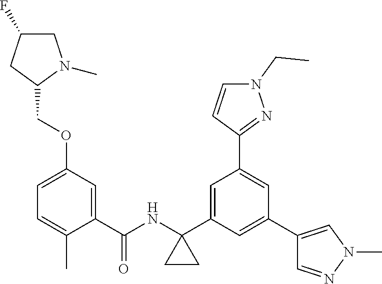

(Method A) White solid, 81% yield. ¹H NMR (400 MHz, DMSO-d₆) δ 9.05 (s, 1H), 8.20 (s, 1H), 7.89 (s, 1H), 7.79 (s, 1H), 7.76 (s, 1H), 7.64 (s, 1H), 7.27-7.18 (m, 2H), 7.01 (d, J=7.9 Hz, 2H), 6.73 (s, 1H), 5.49 (dt, J=52.1, 4.4 Hz, 3H), 5.23-4.91 (m, 1H), 4.40 (dd, J=11.7, 3.4 Hz, 1H), 4.26 (t, J=9.8 Hz, 1H), 4.17 (q, J=7.3 Hz, 2H), 4.01-3.90 (m, 1H), 3.88 (s, 3H), 3.71 (s, 1H), 3.48 (dd, J=37.8, 13.3 Hz, 1H), 3.04 (s, 2H), 2.86 (s, 1H), 2.84-2.68 (m, 1H), 2.32 (s, 3H), 2.19-2.02 (m, 1H), 1.42 (t, J=7.3 Hz, 3H), 1.37 (s, 2H), 1.29 (d, J=5.6 Hz, 2H). ¹³C NMR (101 MHz, DMSO-d₆) δ 169.74, 169.67, 155.67, 154.34, 150.36, 144.75, 144.73, 138.73, 138.69, 136.49, 134.29, 133.21, 132.04, 131.13, 128.46, 128.42, 122.52, 120.42, 120.21, 119.79, 117.99, 115.79, 115.07, 114.04, 102.90, 92.53, 90.79, 67.07, 66.29, 46.81, 41.19, 39.09, 34.85, 34.82, 18.92, 18.39, 16.00. C₃₂H₃₇FN₆O₂, HRMS calculated for m/z [M+H]⁺: 557.3040 (calculated), 557.3045 (found).

### Example 6: N-(1-(3-(1-ethyl-1H-pyrazol-3-yl)-5-(1-methyl-1H-pyrazol-4-yl)phenyl)cyclopropyl)-5-(((2S,4R)-4-fluoro-1-methylpyrrolidin-2-yl)methoxy)-2-methylbenzamide

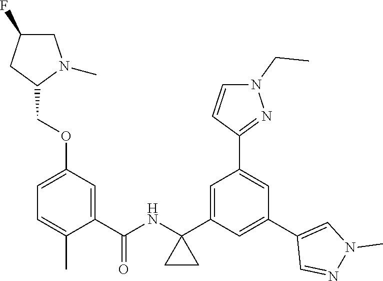

(Method A) White solid, 83% yield. ¹H NMR (400 MHz, DMSO-d₆) δ 8.98 (s, 1H), 8.13 (s, 1H), 7.82 (s, 1H), 7.72 (s, 1H), 7.68 (s, 1H), 7.58 (d, J=6.0 Hz, 1H), 7.15 (d, J=8.5 Hz, 2H), 6.98-6.90 (m, 2H), 6.66 (s, 1H), 5.49 (s, 2H), 4.72 (s, 1H), 4.39-4.16 (m, 1H), 4.10 (q, J=7.3 Hz, 2H), 3.81 (s, 3H), 3.65 (d, J=14.4 Hz, 1H), 3.26 (d, J=33.9 Hz, 1H), 2.98 (s, 1H), 2.82 (s, 2H), 2.25 (s, 3H), 1.96-1.59 (m, 1H), 1.35 (t, J=7.3 Hz, 3H), 1.30 (s, 2H), 1.23 (s, 2H). ¹³C NMR (101 MHz, DMSO-d₆) δ 169.59, 150.36, 144.72, 138.81, 136.49, 134.29, 134.26, 133.24, 132.23, 131.13, 129.12, 128.46, 122.52, 120.22, 120.15, 119.76, 102.88, 54.67, 46.80, 43.66, 39.09, 34.85, 34.81, 18.92, 18.89, 18.49, 15.99. C₃₂H₃₇FN₆O₂, HRMS calculated for m/z [M+H]⁺: 557.3040 (calculated), 557.3046 (found).

### Example 7: N-(1-(3-(1-ethyl-1H-pyrazol-3-yl)-5-(1-methyl-1H-pyrazol-4-yl)phenyl)cyclopropyl)-2-methyl-5-(2-methyl-2,7-diazaspiro[3.5]nonan-7-yl)benzamide

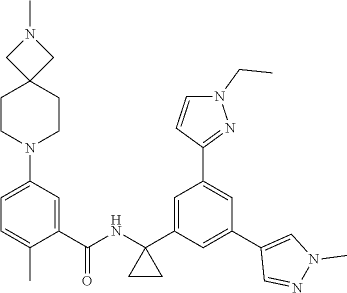 [SMILES: CCn1ccc(-c2cc(-c3cnn(C)c3)cc(C3(NC(=O)c4cc(N5CCC6(CC5)CN(C)C6)ccc4C)CC3)c2)n1]

(Method A) White solid, 69% yield. 1H NMR (400 MHz, DMSO-d₆) δ 8.90 (s, 1H), 8.12 (s, 1H), 7.81 (s, 1H), 7.72 (d, J=2.2 Hz, 1H), 7.67 (s, 1H), 7.56 (d, J=1.7 Hz, 1H), 7.17-7.13 (m, 1H), 7.05 (d, J=9.0 Hz, 1H), 6.93 (d, J=2.4 Hz, 1H), 6.65 (d, J=2.3 Hz, 1H), 4.10 (q, J=7.4 Hz, 2H), 3.96 (dd, J=10.6, 6.5 Hz, 2H), 3.81 (s, 3H), 3.74 (dd, J=10.8, 6.5 Hz, 2H), 3.12 (t, J=5.4 Hz, 2H), 3.03 (t, J=5.5 Hz, 2H), 2.80 (d, J=4.9 Hz, 3H), 2.19 (s, 3H), 1.90-1.81 (m, 4H), 1.35 (t, J=7.3 Hz, 3H), 1.29 (q, J=5.5, 3.9 Hz, 2H), 1.22 (qd, J=5.7, 3.8 Hz, 2H). ¹³C NMR (101 MHz, DMSO-d₆) δ 170.23, 158.95, 158.60, 150.40, 144.89, 138.07, 136.46, 134.26, 133.17, 131.58, 131.09, 129.26, 128.42, 122.54, 120.29, 119.72, 118.08, 115.76, 102.86, 64.45, 46.81, 46.66, 42.10, 34.75, 34.60, 33.72, 33.04, 18.88, 18.51, 16.00. C₃₄H₄₂N₇O, HRMS calculated for m/z [M+H]⁺. 564.3373 (calculated), 564.3375 (found).

### Example 8: N-(1-(3-(1-ethyl-1H-pyrazol-3-yl)-5-(1-methyl-1H-pyrazol-4-yl)phenyl)cyclopropyl)-2-methyl-5-(5-methylhexahydropyrrolo[3,4-c]pyrrol-2(1H)-yl)benzamide

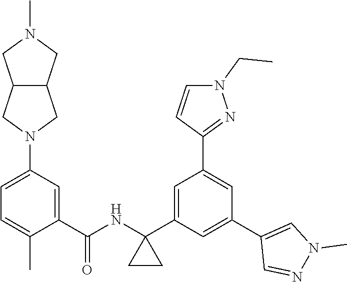 [SMILES: CCn1ccc(-c2cc(-c3cnn(C)c3)cc(C3(NC(=O)c4cc(N5CC6CN(C)CC6C5)ccc4C)CC3)c2)n1]

(Method A) White solid, 78% yield. ¹H NMR (400 MHz, DMSO-d₆) δ 8.89 (s, 1H), 8.12 (s, 1H), 7.81 (s, 1H), 7.72 (s, 1H), 7.68 (s, 1H), 7.56 (s, 1H), 7.17 (s, 1H), 7.01 (d, J=8.1 Hz, 1H), 6.65 (d, J=2.5 Hz, 2H), 6.64-6.59 (m, 1H), 4.10 (q, J=7.3 Hz, 3H), 3.81 (s, 5H), 3.41 (d, J=9.5 Hz, 2H), 3.27 (d, J=7.0 Hz, 2H), 3.18 (d, J=6.2 Hz, 2H), 3.05-2.93 (m, 2H), 2.79 (d, J=3.5 Hz, 2H), 2.77-2.72 (m, 2H), 2.18 (s, 3H), 1.34 (t, J=7.2 Hz, 3H), 1.30 (d, J=7.9 Hz, 2H), 1.22 (d, J=5.4 Hz, 2H). ¹³C NMR (101 MHz, DMSO-d₆) δ 170.45, 150.39, 144.91, 138.12, 136.46, 134.26, 133.14, 131.46, 131.11, 128.43, 122.54, 120.10, 119.72, 113.42, 102.89, 61.10, 60.38, 54.16, 52.75, 46.80, 40.94, 39.09, 34.73, 18.81, 18.50, 16.00. C₃₃H₃₉N₇O, HRMS calculated for m/z [M+H]⁺: 550.3294 (calculated), 550.3300 (found).

### Example 9: 4-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide

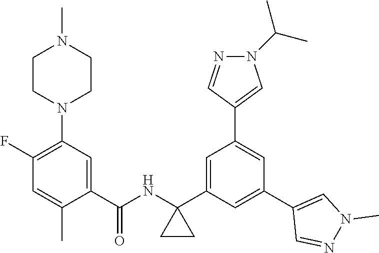 [SMILES: Cc1cc(F)c(N2CCN(C)CC2)cc1C(=O)NC1(c2cc(-c3cnn(C)c3)cc(-c3cnn(C(C)C)c3)c2)CC1]

(Method A) White solid, 79% yield. ¹H NMR (400 MHz, DMSO-d₆) δ 8.92 (s, 1H), 8.17 (s, 1H), 8.07 (s, 1H), 7.80 (s, 2H), 7.55 (s, 1H), 7.16 (s, 2H), 7.03 (dd, J=14.7, 11.2 Hz, 2H), 4.48-4.38 (m, 1H), 3.81 (s, 3H), 3.47 (d, J=11.7 Hz, 4H), 3.16 (d, J=12.4 Hz, 2H), 2.97 (t, J=11.8 Hz, 2H), 2.81 (s, 3H), 2.23 (s, 3H), 1.39 (d, J=6.6 Hz, 6H), 1.30 (d, J=4.2 Hz, 2H), 1.25-1.20 (m, 2H). ¹³C NMR (101 MHz, DMSO-d₆) δ 169.10, 144.97, 136.44, 135.92, 135.84, 135.79, 135.70, 133.52, 133.23, 128.33, 125.17, 124.04, 122.60, 122.05, 119.77, 119.61, 119.52, 119.24, 118.69, 118.49, 53.61, 52.98, 47.67, 42.66, 39.11, 34.92, 23.14, 19.02, 18.38.

C₃₂H₃₈FN₇O, HRMS calculated for m/z [M+H]⁺: 556.3200 (calculated), 556.3205 (found).

### Example 10: N-(1-(3-(1-ethyl-1H-pyrazol-3-yl)-5-(1-methyl-1H-pyrazol-4-yl)phenyl)cyclopropyl)-4-fluoro-2-methyl-5-(4-methylpiperazin-1-yl)benzamide

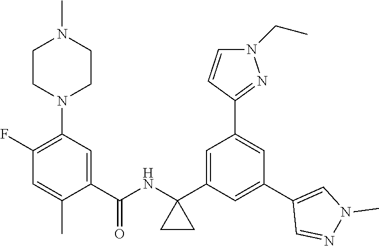 [SMILES: CCn1ccc(-c2cc(-c3cnn(C)c3)cc(C3(NC(=O)c4cc(N5CCN(C)CC5)c(F)cc4C)CC3)c2)n1]

(Method A) White solid, 43% yield. ¹H NMR (400 MHz, DMSO-d₆) δ 9.04 (s, 1H), 8.19 (s, 1H), 7.88 (s, 1H), 7.78 (d, J=2.2 Hz, 1H), 7.75 (s, 1H), 7.63 (s, 1H), 7.23 (s, 1H), 7.15-7.05 (m, 2H), 6.72 (d, J=2.3 Hz, 1H), 4.62 (s, 4H), 4.17 (q, J=7.3 Hz, 2H), 3.88 (s, 3H), 3.26 (d, J=12.4 Hz, 2H), 3.03 (t, J=11.5 Hz, 2H), 2.88 (s, 3H), 2.32 (s, 3H), 1.41 (t, J=7.3 Hz, 3H), 1.36 (d, J=4.2 Hz, 2H), 1.33-1.29 (m, 2H). ¹³C NMR (101 MHz, DMSO-d₆) δ 169.10, 150.36, 144.74, 136.47, 135.75, 135.66, 134.28, 133.85, 133.22, 132.14, 132.06, 131.12, 128.45, 122.51, 120.35, 120.16, 119.78, 119.25, 118.69, 118.48, 117.69, 114.79, 102.88, 52.99, 47.69, 46.81, 42.67, 39.08, 34.87, 19.05, 18.41, 15.99. C₃₁H₃₆FN₇O, HRMS calculated for m/z [M+H]⁺: 542.3044 (calculated), 542.3051 (found).

### Example 11: 3-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide

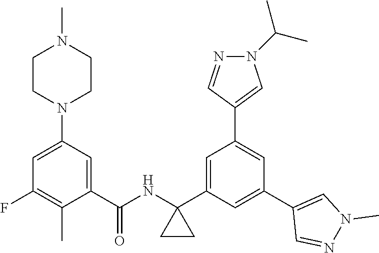 [SMILES: Cc1c(F)cc(N2CCN(C)CC2)cc1C(=O)NC1(c2cc(-c3cnn(C)c3)cc(-c3cnn(C(C)C)c3)c2)CC1]

(Method A) White solid, 80% yield. ¹H NMR (400 MHz, DMSO-d₆) δ 9.03 (s, 1H), 8.24 (s, 1H), 8.14 (s, 1H), 7.87 (s, 2H), 7.62 (s, 1H), 7.24 (s, 2H), 6.95 (dd, J=12.9, 2.5 Hz, 1H), 6.84 (d, J=2.5 Hz, 1H), 4.55-4.43 (m, 1H), 4.44 (s, 4H), 3.88 (s, 3H), 3.53 (d, J=12.0 Hz, 2H), 3.01 (t, J=12.6 Hz, 2H), 2.87 (s, 3H), 2.15 (s, 3H), 1.46 (d, J=6.7 Hz, 6H), 1.37 (d, J=4.2 Hz, 2H), 1.32-1.27 (m, 2H). ¹³C NMR (101 MHz, DMSO-d₆) δ 169.04, 160.57, 148.98, 148.87, 144.86, 140.16, 136.47, 135.95, 135.84, 133.52, 133.25, 128.34, 125.17, 124.04, 122.60, 122.05, 119.79, 119.62, 119.57, 113.83, 113.23, 113.05, 110.69, 53.61, 53.29, 52.50, 45.77, 42.56, 39.11, 34.94, 23.14, 18.32, 10.90, 10.86. C₃₂H₃₈FN₇O, HRMS calculated for m/z [M+H]⁺: 556.3200 (calculated), 556.3207 (found).

### Example 12: N-(1-(3-(1-ethyl-1H-pyrazol-3-yl)-5-(1-methyl-1H-pyrazol-4-yl)phenyl)cyclopropyl)-3-fluoro-2-methyl-5-(4-methylpiperazin-1-yl)benzamide

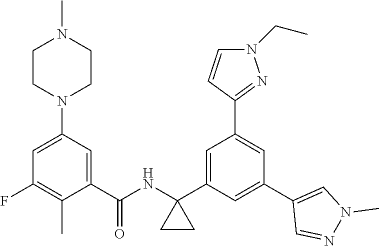 [SMILES: CCn1ccc(-c2cc(-c3cnn(C)c3)cc(C3(NC(=O)c4cc(N5CCN(C)CC5)cc(F)c4C)CC3)c2)n1]

(Method A) White solid, 68% yield. ¹H NMR (400 MHz, DMSO-d₆) δ 9.01 (s, 1H), 8.12 (s, 1H), 7.82 (s, 1H), 7.72 (d, J=2.3 Hz, 1H), 7.68 (s, 1H), 7.57 (s, 1H), 7.16 (s, 1H), 6.88 (dd, J=13.0, 2.5 Hz, 1H), 6.75 (s, 1H), 6.65 (d, J=2.3 Hz, 1H), 4.10 (q, J=7.3 Hz, 2H), 3.83 (d, J=10.7 Hz, 2H), 3.81 (s, 3H), 3.46 (d, J=11.9 Hz, 2H), 2.93 (t, J=12.5 Hz, 2H), 2.80 (s, 3H), 2.10 (s, 3H), 1.34 (t, J=7.3 Hz, 3H), 1.32-1.27 (m, 2H), 1.26-1.22 (m, 2H). ¹³C NMR (101 MHz, DMSO-d₆) δ 169.03, 162.96, 160.57, 159.09, 158.74, 150.36, 148.94, 148.83, 144.65, 140.18, 140.13, 136.49, 134.29, 133.24, 131.13, 128.46, 122.51, 120.35, 120.17, 119.81, 113.35, 113.17, 110.69, 104.24, 103.96, 102.90, 52.48, 46.81, 45.78, 42.55, 39.09, 34.89, 18.37, 15.98, 10.89, 10.85. C₃₁H₃₆FN₇O, HRMS calculated for m/z [M+H]⁺. 542.3044 (calculated), 542.3050 (found).

### Example 13: (S)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide

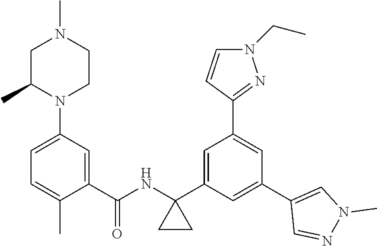

(Method A) White solid, 69% yield. ¹H NMR (400 MHz, DMSO-d₆) δ 8.90 (s, 1H), 8.12 (s, 1H), 7.81 (s, 1H), 7.72 (d, J=2.3 Hz, 1H), 7.68 (d, J=1.7 Hz, 1H), 7.56 (s, 1H), 7.17 (s, 1H), 7.06 (d, J=8.7 Hz, 1H), 6.93-6.79 (m, 2H), 6.65 (d, J=2.3 Hz, 1H), 4.28 (s, 1H), 4.10 (q, J=7.3 Hz, 2H), 3.81 (s, 3H), 3.51 (d, J=12.0 Hz, 1H), 3.43 (t, J=8.9 Hz, 2H), 3.22 (s, 1H), 3.08 (d, J=12.8 Hz, 2H), 2.80 (s, 3H), 2.27 (s, 1H), 2.21 (s, 2H), 1.34 (t, J=7.2 Hz, 3H), 1.29 (s, 2H), 1.24 (d, J=3.7 Hz, 2H), 1.01 (d, J=6.9 Hz, 2H), 0.83 (d, J=6.0 Hz, 1H). ¹³C NMR (101 MHz, DMSO-d₆) δ 170.22, 150.38, 136.46, 134.27, 133.18, 131.77, 131.09, 128.44, 122.54, 120.35, 120.16, 119.74, 102.87, 57.27, 53.08, 48.87, 46.81, 43.41, 39.09, 34.80, 18.88, 18.49, 16.00, 11.38. C₃₂H₃₉N₇O, HRMS calculated for m/z [M+H]⁺: 538.3294 (calculated), 538.3301 (found).

### Example 14: N-(1-(3-(1-ethyl-1H-pyrazol-3-yl)-5-(1-methyl-1H-pyrazol-4-yl)phenyl)cyclopropyl)-5-(4-isopropylpiperazin-1-yl)-2-methylbenzamide

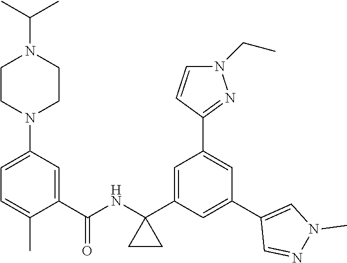 [SMILES: CCn1ccc(-c2cc(-c3cnn(C)c3)cc(C3(NC(=O)c4cc(N5CCN(C(C)C)CC5)ccc4C)CC3)c2)n1]

(Method A) White solid, 77% yield. ¹H NMR (400 MHz, DMSO-d₆) δ 8.93 (s, 1H), 8.12 (s, 1H), 7.81 (s, 1H), 7.71 (d, J=2.2 Hz, 1H), 7.68 (s, 1H), 7.57 (s, 1H), 7.18 (s, 1H), 7.08 (d, J=8.3 Hz, 1H), 6.93 (dd, J=8.3, 2.7 Hz, 1H), 6.90 (d, J=2.6 Hz, 1H), 6.65 (s, 1H), 4.10 (q, J=7.2 Hz, 2H), 3.81 (s, 5H), 3.49 (d, J=5.0 Hz, 1H), 3.45 (d, J=11.9 Hz, 2H), 3.08 (q, J=11.1 Hz, 2H), 2.91 (t, J=12.4 Hz, 2H), 2.22 (s, 3H), 1.34 (t, J=7.2 Hz, 3H), 1.29 (t, J=3.1 Hz, 2H), 1.23 (d, J=6.4 Hz, 8H). ¹³C NMR (101 MHz, DMSO-d₆) δ 170.16, 150.39, 147.72, 144.85, 138.13, 136.47, 134.28, 133.18, 131.71, 131.11, 128.45, 127.28, 122.55, 120.41, 120.18, 119.76, 117.55, 115.34, 102.88, 57.55, 47.81, 46.81, 46.53, 39.09, 34.82, 18.91, 18.45, 16.92, 15.98. C₃₃H₄₁N₇O, HRMS calculated for m/z [M+H]⁺: 552.3451 (calculated), 554.3459z (found).

### Example 15: 5-(4-cyclopropylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide

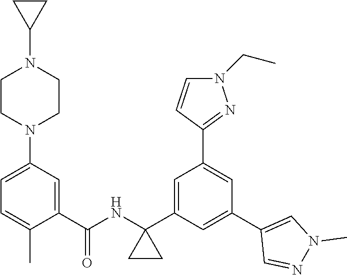 [SMILES: CCn1ccc(-c2cc(-c3cnn(C)c3)cc(C3(NC(=O)c4cc(N5CCN(C6CC6)CC5)ccc4C)CC3)c2)n1]

(Method A) White solid, 80% yield. ¹H NMR (400 MHz, DMSO-d₆) δ 8.99 (s, 1H), 8.19 (s, 1H), 7.88 (s, 1H), 7.79 (s, 1H), 7.75 (s, 1H), 7.64 (s, 1H), 7.25 (s, 1H), 7.15 (d, J=8.3 Hz, 1H), 7.03-6.97 (m, 2H), 6.72 (s, 1H), 4.17 (q, J=7.3 Hz, 2H), 3.88 (s, 3H), 3.48 (s, 4H), 2.97 (tt, J=7.5, 3.9 Hz, 2H), 2.29 (s, 3H), 1.42 (t, J=7.2 Hz, 3H), 1.36 (t, J=3.1 Hz, 2H), 1.33-1.28 (m, 2H), 1.02 (q, J=6.0, 5.2 Hz, 2H), 0.86 (t, J=6.8 Hz, 2H). ¹³C NMR (101 MHz, DMSO-d₆) δ 170.15, 150.39, 147.68, 144.85, 138.14, 136.47, 134.28, 133.18, 131.72, 131.11, 128.44, 127.23, 122.55, 120.43, 120.21, 119.77, 117.54, 115.36, 102.88, 52.24, 46.81, 46.29, 39.09, 38.84, 34.83, 18.91, 18.44, 15.98, 3.85. C₃₃H₃₉N₇O, HRMS calculated for m/z [M+H]⁺: 550.3294 (calculated), 550.3301 (found).

### Example 16: N-(1-(3-(1-isopropyl-1H-pyrazol-4-yl)-5-(1-methyl-1H-pyrazol-4-yl)phenyl)cyclopropyl)-5-(4-isopropylpiperazin-1-yl)-2-methylbenzamide

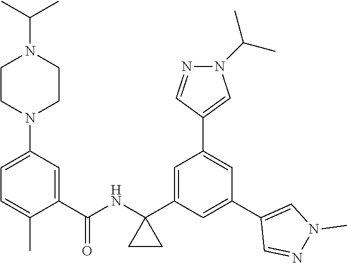 [SMILES: Cc1ccc(N2CCN(C(C)C)CC2)cc1C(=O)NC1(c2cc(-c3cnn(C)c3)cc(-c3cnn(C(C)C)c3)c2)CC1]

(Method A) White solid, 77% yield. ¹H NMR (400 MHz, DMSO-d₆) δ 8.87 (s, 1H), 8.17 (s, 1H), 8.08 (s, 1H), 7.82 (s, 1H), 7.79 (s, 2H), 7.55 (s, 1H), 7.51 (s, 1H), 7.18 (s, 2H), 6.92 (d, J=3.5 Hz, 1H), 4.48-4.42 (m, 1H), 4.40-4.35 (m, 5H), 3.81 (s, 3H), 3.11 (t, J=10.7 Hz, 2H), 2.91 (t, J=12.3 Hz, 2H), 2.20 (s, 3H), 1.40 (d, J=6.6 Hz, 6H), 1.30 (t, J=3.4 Hz, 2H), 1.24 (d, J=6.6 Hz, 6H), 1.22 (d, J=2.3 Hz, 2H). ¹³C NMR (101 MHz, DMSO-d₆) δ 170.14, 147.73, 145.06, 138.16, 136.45, 135.92, 135.85, 133.48, 133.23, 131.72, 128.33, 127.12, 125.16, 124.03, 122.64, 122.09, 119.74, 119.59, 119.57, 117.55, 115.31, 113.84, 57.58, 53.61, 53.28, 47.84, 46.55, 39.13, 34.85, 23.16, 18.86, 18.41, 16.96. C₃₄H₄₃N₇O, HRMS calculated for m/z [M+H]⁺: 566.3607 (calculated), 566.3612 (found).

### Example 17: 5-(4-cyclopropylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide

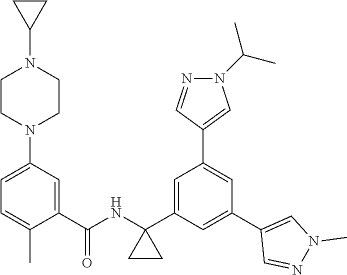 [SMILES: Cc1ccc(N2CCN(C3CC3)CC2)cc1C(=O)NC1(c2cc(-c3cnn(C)c3)cc(-c3cnn(C(C)C)c3)c2)CC1]

(Method A) White solid, 90% yield. ¹H NMR (400 MHz, DMSO-d₆) δ 8.87 (s, 1H), 8.17 (s, 1H), 8.07 (s, 1H), 7.79 (s, 2H), 7.55 (s, 1H), 7.18 (s, 2H), 7.08 (d, J=8.3 Hz, 1H), 6.92 (d, J=7.2 Hz, 2H), 4.70 (s, 4H), 4.44 (p, J=6.6 Hz, 1H), 3.81 (s, 3H), 3.80-3.75 (m, 1H), 3.42 (s, 4H), 2.90 (tt, J=7.6, 3.9 Hz, 1H), 2.20 (s, 3H), 1.39 (d, J=6.7 Hz, 6H), 1.31 (q, J=5.1, 4.2 Hz, 2H), 1.22 (q, J=5.6, 4.8 Hz, 2H), 0.94 (q, J=6.0, 5.1 Hz, 2H), 0.79 (t, J=6.9 Hz, 2H). ¹³C NMR (101 MHz, DMSO-d₆) δ 170.15, 147.71, 145.07, 138.15, 136.46, 135.93, 133.48, 133.23, 131.73, 128.33, 127.04, 125.16, 122.65, 122.10, 119.74, 119.60, 117.50, 115.31, 53.61, 52.24, 46.28, 39.12, 38.84, 34.86, 23.15, 18.86, 18.42, 3.85. C₃₄H₄₁N₇O, HRMS calculated for m/z [M+H]⁺. 564.3451 (calculated), 564.3457 (found).

### Example 18: (R)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide

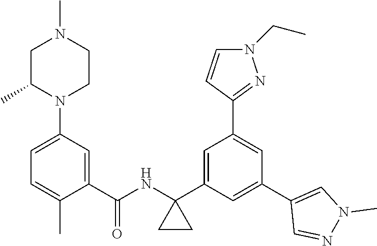

(Method A) White solid, 78% yield. ¹H NMR (400 MHz, DMSO-d₆) δ 9.88 (s, 1H), 8.95 (d, J=37.5 Hz, 1H), 8.12 (s, 1H), 7.81 (s, 1H), 7.72 (d, J=2.2 Hz, 1H), 7.68 (s, 1H), 7.56 (s, 1H), 7.16 (s, 1H), 7.06 (d, J=8.5 Hz, 1H), 6.90-6.82 (m, 1H), 6.64 (d, J=2.2 Hz, 1H), 4.94 (s, 4H), 4.27 (d, J=7.4 Hz, 1H), 4.10 (q, J=7.3 Hz, 2H), 3.81 (s, 3H), 3.48-3.39 (m, 2H), 3.13-3.06 (m, 1H), 2.80 (s, 3H), 2.21 (s, 2H), 1.34 (t, J=7.3 Hz, 3H), 1.29 (s, 2H), 1.23 (q, J=5.5, 4.6 Hz, 2H), 0.92 (dd, J=71.0, 6.4 Hz, 3H). ¹³C NMR (101 MHz, DMSO-d₆) δ 170.23, 150.39, 146.41, 144.87, 136.47, 134.27, 133.19, 131.78, 131.11, 128.44, 122.54, 120.35, 120.16, 119.74, 118.11, 117.39, 115.19, 114.97, 102.87, 57.28, 48.88, 46.81, 43.41, 39.09, 34.80, 18.90, 18.49, 16.00, 11.37. C₃₂H₃₉N₇O, HRMS calculated for m/z [M+H]⁺: 538.3294 (calculated), 538.3302 (found).

### Example 19: N-(1-(3-(1-ethyl-1H-pyrazol-3-yl)-5-(1-methyl-1H-pyrazol-4-yl)phenyl)cyclopropyl)-2-fluoro-6-methyl-3-(4-methylpiperazin-1-yl)benzamide

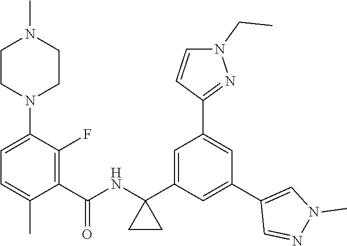 [SMILES: CCn1ccc(-c2cc(-c3cnn(C)c3)cc(C3(NC(=O)c4c(C)ccc(N5CCN(C)CC5)c4F)CC3)c2)n1]

(Method A) White solid, 61% yield. 1H NMR (400 MHz, DMSO-d₆) δ 8.17 (s, 1H), 7.87 (d, J=3.8 Hz, 2H), 7.76 (d, J=6.2 Hz, 2H), 7.62 (s, 1H), 7.37-7.31 (m, 1H), 7.12 (q, J=7.0, 5.8 Hz, 2H), 6.70 (d, J=2.3 Hz, 1H), 6.11 (t, J=6.8 Hz, 1H), 4.62 (s, 4H), 4.17 (q, J=7.3 Hz, 2H), 3.88 (s, 3H), 3.26 (d, J=12.4 Hz, 2H), 3.03 (t, J=11.5 Hz, 2H), 2.78 (s, 3H), 2.39 (s, 3H), 1.37 (t, J=7.3 Hz, 3H), 1.37 (d, J=4.2 Hz, 2H), 1.33-1.29 (m, 2H). ¹³C NMR (101 MHz, DMSO-d₆) δ 170.48, 163.97, 159.09, 158.74, 149.91, 138.19, 136.52, 134.80, 133.59, 131.33, 128.56, 126.61, 122.24, 122.08, 121.65, 120.71, 102.96, 54.77, 51.86, 49.45, 46.87, 42.65, 39.17, 19.35, 15.96. C₃₁H₃₆FN₇O, HRMS calculated for m/z [M+H]⁺: 542.3044 (calculated), 542.3052 (found).

### Example 20: 5-(4-(tert-butyl)piperazin-1-yl)-N-(1-(3-(1-ethyl-1H-pyrazol-3-yl)-5-(1-methyl-1H-pyrazol-4-yl)phenyl)cyclopropyl)-2-methylbenzamide

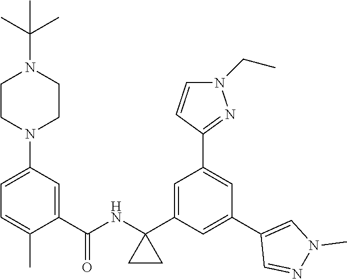 [SMILES: CCn1ccc(-c2cc(-c3cnn(C)c3)cc(C3(NC(=O)c4cc(N5CCN(C(C)(C)C)CC5)ccc4C)CC3)c2)n1]

(Method A) White solid, 77% yield. ¹H NMR (400 MHz, DMSO-d₆) δ 8.95 (s, 1H), 8.13 (s, 1H), 7.81 (s, 1H), 7.72 (d, J=2.3 Hz, 1H), 7.68 (s, 1H), 7.57 (s, 1H), 7.18 (s, 1H), 7.08 (d, J=8.4 Hz, 1H), 6.93 (dd, J=8.3, 2.7 Hz, 1H), 6.89 (d, J=2.7 Hz, 1H), 6.65 (d, J=2.3 Hz, 1H), 4.10 (q, J=7.2 Hz, 2H), 3.81 (s, 3H), 3.79 (d, J=9.9 Hz, 2H), 3.53 (d, J=11.5 Hz, 2H), 3.07 (q, J=11.1 Hz, 2H), 2.93 (t, J=12.1 Hz, 2H), 2.22 (s, 3H), 1.34 (t, J=7.3 Hz, 3H), 1.30 (s, 9H), 1.29 (s, 2H), 1.25-1.20 (m, 2H). ¹³C NMR (101 MHz, DMSO-d₆) δ 170.17, 150.40, 147.65, 144.85, 138.18, 136.48, 134.30, 133.19, 131.72, 131.10, 128.44, 127.27, 122.56, 121.13, 120.47, 120.21, 119.79, 118.20, 117.45, 115.28, 102.89, 63.32, 63.30, 46.81, 46.73, 46.10, 39.10, 34.84, 24.32, 18.90, 18.41, 15.97. C₃₄H₄₃N₇O, HRMS calculated for m/z [M+H]⁺: 566.3607 (calculated), 566.3611 (found).

### Example 21: (S)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide

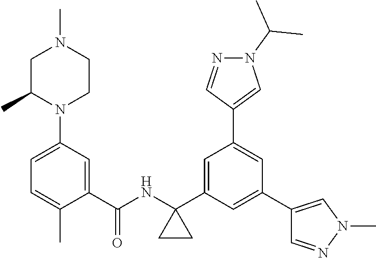

(Method A) White solid, 44% yield. ¹H NMR (400 MHz, DMSO-d₆) δ 9.77 (s, 1H), 8.91 (d, J=40.6 Hz, 1H), 8.17 (s, 1H), 8.07 (s, 1H), 7.79 (s, 2H), 7.55 (d, J=1.6 Hz, 1H), 7.17 (d, J=1.6 Hz, 2H), 7.06 (d, J=7.9 Hz, 1H), 6.87 (d, J=7.7 Hz, 1H), 4.48-4.39 (m, 1H), 4.29 (s, 1H), 3.81 (s, 3H), 3.43 (d, J=13.6 Hz, 2H), 3.27-3.00 (m, 3H), 2.80 (s, 3H), 2.19 (s, 2H), 1.39 (d, J=6.7 Hz, 6H), 1.34 (d, J=6.7 Hz, 2H), 1.31 (s, 2H), 1.22 (t, J=3.3 Hz, 2H), 0.92 (dd, J=69.9, 6.1 Hz, 3H). ¹³C NMR (101 MHz, DMSO-d₆) δ 170.22, 146.42, 145.08, 136.43, 135.91, 135.83, 133.49, 133.21, 131.78, 128.33, 125.15, 124.03, 122.63, 122.08, 119.71, 119.56, 119.49, 114.91, 113.83, 57.28, 53.61, 53.28, 48.82, 43.41, 39.12, 34.83, 23.15, 18.85, 18.48, 11.38. C₃₃H₄₁N₇O, HRMS calculated for m/z [M+H]⁺: 552.3451 (calculated), 552.3459 (found).

### Example 22: (S)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide

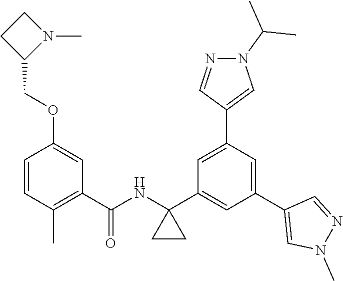

(Method A) White solid, 79% yield. ¹H NMR (400 MHz, DMSO-d₆) δ 8.92 (s, 1H), 8.18 (s, 1H), 8.08 (s, 1H), 7.80 (s, 2H), 7.55 (s, 1H), 7.17 (d, J=3.8 Hz, 2H), 7.14 (d, J=8.1 Hz, 1H), 6.95-6.85 (m, 2H), 4.62 (d, J=5.9 Hz, 1H), 4.44 (p, J=6.7 Hz, 1H), 4.27 (d, J=5.4 Hz, 1H), 4.04-3.83 (m, 1H), 3.81 (s, 3H), 3.65 (s, 1H), 3.33-3.03 (m, 1H), 2.90-2.75 (m, 3H), 2.34 (qd, J=8.2, 7.2, 3.8 Hz, 1H), 2.22 (d, J=1.6 Hz, 3H), 2.20-1.93 (m, 1H), 1.39 (d, J=6.6 Hz, 6H), 1.33-1.28 (m, 2H), 1.22 (d, J=3.8 Hz, 2H). ¹³C NMR (101 MHz, DMSO-d₆) δ 169.67, 155.81, 154.41, 144.98, 138.74, 138.69, 136.45, 135.92, 133.50, 133.25, 132.03, 128.35, 128.19, 125.16, 122.62, 122.08, 119.78, 119.60, 117.83, 114.93, 114.08, 68.25, 67.10, 53.61, 53.06, 41.46, 39.11, 23.15, 18.86, 18.36. C₃₂H₃₈N₆O₂, HRMS calculated for m/z [M+H]⁺. 539.3134 (calculated), 539.3141 (found).

### Example 23: N-(1-(3-(1-(cyclopropylmethyl)-1H-pyrazol-4-yl)-5-(1-ethyl-1H-pyrazol-3-yl)phenyl)cyclopropyl)-2-methyl-5-(4-methylpiperazin-1-yl)benzamide

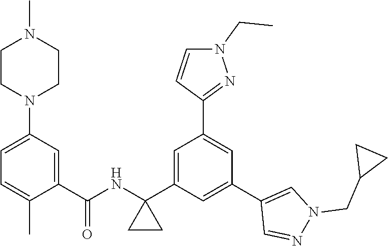 [SMILES: CCn1ccc(-c2cc(-c3cnn(CC4CC4)c3)cc(C3(NC(=O)c4cc(N5CCN(C)CC5)ccc4C)CC3)c2)n1]

(Method A) White solid, 70% yield. ¹H NMR (400 MHz, DMSO-d₆) δ 8.91 (s, 1H), 8.18 (s, 1H), 7.82 (s, 1H), 7.72 (d, J=2.2 Hz, 1H), 7.69 (s, 1H), 7.57 (s, 1H), 7.18 (s, 1H), 7.07 (d, J=8.2 Hz, 1H), 6.92 (d, J=8.6 Hz, 2H), 6.65 (d, J=2.3 Hz, 1H), 4.10 (q, J=7.3 Hz, 2H), 3.92 (d, J=7.1 Hz, 2H), 3.76 (d, J=13.1 Hz, 2H), 3.46 (d, J=11.9 Hz, 2H), 3.15-3.03 (m, 2H), 2.89 (t, J=12.5 Hz, 2H), 2.80 (s, 3H), 2.22 (s, 3H), 1.34 (t, J=7.3 Hz, 3H), 1.32-1.27 (m, 2H), 1.24 (dd, J=5.8, 2.3 Hz, 2H), 1.20 (dt, J=9.2, 3.1 Hz, 1H), 0.51-0.45 (m, 2H), 0.33 (q, J=5.1, 4.5 Hz, 2H). ¹³C NMR (101 MHz, DMSO-d₆) δ 170.14, 150.42, 147.70, 144.85, 138.12, 136.33, 134.26, 133.27, 131.70, 131.09, 127.21, 127.18, 122.38, 120.44, 120.21, 119.76, 117.58, 115.35, 102.90, 56.35, 52.75, 46.81, 46.36, 42.57, 34.84, 18.93, 18.48, 16.01, 11.98, 4.06. C₃₄H₄₁N₇O, HRMS calculated for m/z [M+H]⁺: 564.3451 (calculated), 564.3459 (found).

### Example 24: N-(1-(3-(1-(difluoromethyl)-1H-pyrazol-3-yl)-5-(1-ethyl-1H-pyrazol-3-yl)phenyl)cyclopropyl)-2-methyl-5-(4-methylpiperazin-1-yl)benzamide

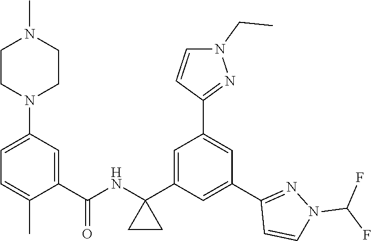 [SMILES: CCn1ccc(-c2cc(-c3ccn(C(F)F)n3)cc(C3(NC(=O)c4cc(N5CCN(C)CC5)ccc4C)CC3)c2)n1]

(Method A) White solid, 72% yield. ¹H NMR (400 MHz, DMSO-d₆) δ 8.98 (s, 1H), 8.25 (d, J=2.7 Hz, 1H), 8.01 (s, 1H), 7.74 (d, J=2.3 Hz, 1H), 7.67 (s, 1H), 7.60 (s, 1H), 7.07 (d, J=8.3 Hz, 1H), 7.02 (d, J=2.7 Hz, 1H), 6.92 (dd, J=10.9, 2.7 Hz, 2H), 6.68 (d, J=2.3 Hz, 1H), 4.12 (q, J=7.3 Hz, 2H), 3.75 (d, J=13.0 Hz, 2H), 3.46 (d, J=12.0 Hz, 2H), 3.07 (d, J=16.8 Hz, 2H), 2.89 (t, J=12.4 Hz, 2H), 2.80 (s, 3H), 2.21 (s, 3H), 1.35 (t, J=7.3 Hz, 3H), 1.32-1.29 (m, 2H), 1.28-1.25 (m, 2H), 1.25-1.20 (m, 1H). ¹³C NMR (101 MHz, DMSO-d₆) δ 170.20, 154.04, 150.05, 147.70, 145.03, 138.04, 134.43, 132.44, 131.71, 131.30, 127.27, 122.47, 121.55, 120.44, 117.63, 115.32, 110.76, 106.10, 102.97, 52.74, 46.86, 46.36, 42.57, 34.84, 18.88, 18.48, 16.02. C₃₁H₃₅F₂N₇O, HRMS calculated for m/z [M+H]⁺: 560.2949 (calculated), 560.2953 (found).

### Example 25: N-(1-(3-(1-(2,2-difluoroethyl)-1H-pyrazol-4-yl)-5-(1-ethyl-1H-pyrazol-3-yl)phenyl)cyclopropyl)-2-methyl-5-(4-methylpiperazin-1-yl)benzamide

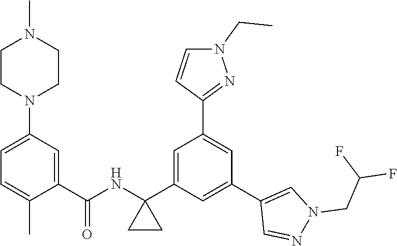 [SMILES: CCn1ccc(-c2cc(-c3cnn(CC(F)F)c3)cc(C3(NC(=O)c4cc(N5CCN(C)CC5)ccc4C)CC3)c2)n1]

(Method A) White solid, 58% yield. ¹H NMR (400 MHz, DMSO-d₆) δ 8.92 (s, 1H), 8.22 (s, 1H), 7.95 (s, 1H), 7.71 (d, J=3.0 Hz, 2H), 7.59 (s, 1H), 7.19 (s, 1H), 7.07 (d, J=8.2 Hz, 1H), 6.95-6.90 (m, 2H), 6.66 (d, J=2.3 Hz, 1H), 6.34 (m, 1H), 4.59 (td, J=15.1, 3.8 Hz, 2H), 4.10 (q, J=7.3 Hz, 2H), 3.76 (d, J=13.1 Hz, 2H), 3.46 (d, J=12.0 Hz, 2H), 3.07 (d, J=15.0 Hz, 2H), 2.89 (t, J=12.5 Hz, 2H), 2.80 (s, 3H), 2.21 (s, 3H), 1.34 (t, J=7.3 Hz, 3H), 1.30 (dd, J=6.5, 2.5 Hz, 2H), 1.27-1.23 (m, 2H). ¹³C NMR (101 MHz, DMSO-d₆) δ 170.16, 150.32, 147.71, 144.97, 138.10, 137.78, 134.34, 132.70, 131.70, 131.14, 129.16, 127.21, 123.18, 120.57, 120.54, 119.87, 117.59, 115.34, 114.51, 102.91, 52.75, 46.82, 46.36, 42.57, 34.84, 18.91, 18.50, 16.00. C₃₂H₃₇F₂N₇O, HRMS calculated for m/z [M+H]⁺: 574.3106 (calculated), 574.3112 (found).

### Example 26: N-(1-(3-(1-cyclopropyl-1H-pyrazol-4-yl)-5-(1-ethyl-1H-pyrazol-3-yl)phenyl)cyclopropyl)-2-methyl-5-(4-methylpiperazin-1-yl)benzamide

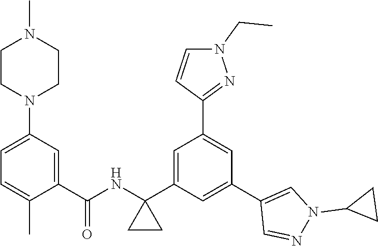 [SMILES: CCn1ccc(-c2cc(-c3cnn(C4CC4)c3)cc(C3(NC(=O)c4cc(N5CCN(C)CC5)ccc4C)CC3)c2)n1]

(Method A) White solid, 70% yield. ¹H NMR (400 MHz, DMSO-d₆) δ 8.91 (s, 1H), 8.22 (s, 1H), 7.81 (s, 1H), 7.72 (d, J=2.3 Hz, 1H), 7.69 (s, 1H), 7.59 (s, 1H), 7.15 (s, 1H), 7.07 (d, J=8.2 Hz, 1H), 6.95-6.89 (m, 2H), 6.65 (d, J=2.3 Hz, 1H), 4.09 (q, J=7.3 Hz, 2H), 3.76 (d, J=13.1 Hz, 2H), 3.68 (tt, J=7.5, 3.9 Hz, 1H), 3.47 (d, J=11.9 Hz, 2H), 3.09 (s, 2H), 2.89 (t, J=12.5 Hz, 2H), 2.80 (s, 3H), 2.22 (s, 3H), 1.34 (t, J=7.3 Hz, 3H), 1.29 (d, J=4.0 Hz, 2H), 1.23 (t, J=6.0 Hz, 2H), 1.02 (q, J=4.2 Hz, 2H), 0.96-0.89 (m, 2H). ¹³C NMR (101 MHz, DMSO-d₆) δ 170.15, 150.42, 147.69, 144.84, 138.11, 136.53, 134.22, 133.10, 131.70, 131.09, 127.52, 127.21, 122.28, 120.26, 119.79, 117.59, 115.34, 102.91, 52.75, 46.81, 46.36, 42.57, 34.82, 33.25, 18.92, 18.55, 16.00, 6.73. C₃₃H₃₉N₇O, HRMS calculated for m/z [M+H]⁺: 550.3294 (calculated), 550.3301 (found).

### Example 27: N-(1-(3-(1-(difluoromethyl)-1H-pyrazol-4-yl)-5-(1-ethyl-1H-pyrazol-3-yl)phenyl)cyclopropyl)-2-methyl-5-(4-methylpiperazin-1-yl)benzamide

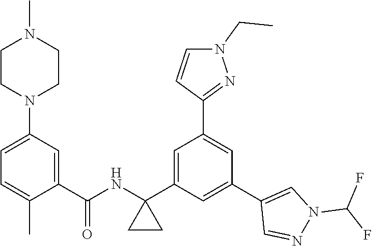 [SMILES: CCn1ccc(-c2cc(-c3cnn(C(F)F)c3)cc(C3(NC(=O)c4cc(N5CCN(C)CC5)ccc4C)CC3)c2)n1]

(Method A) White solid, 74% yield. ¹H NMR (400 MHz, DMSO-d₆) δ 8.93 (s, 1H), 8.74 (s, 1H), 8.26 (s, 1H), 7.81 (s, 1H), 7.78 (s, 1H), 7.73 (t, J=1.8 Hz, 2H), 7.24 (s, 1H), 7.08 (d, J=8.2 Hz, 1H), 6.92 (d, J=7.5 Hz, 2H), 6.70 (d, J=2.3 Hz, 1H), 4.46 (s, 2H), 4.10 (q, J=7.3 Hz, 2H), 3.76 (d, J=13.1 Hz, 2H), 3.47 (d, J=12.0 Hz, 2H), 2.89 (t, J=12.5 Hz, 2H), 2.80 (s, 3H), 2.22 (s, 3H), 1.34 (t, J=7.2 Hz, 5H), 1.28-1.21 (m, 2H). ¹³C NMR (101 MHz, DMSO-d₆) δ 170.21, 150.20, 147.70, 145.16, 140.55, 138.07, 134.38, 131.70, 131.54, 131.18, 127.24, 125.95, 125.12, 121.41, 120.69, 120.38, 117.60, 115.36, 111.08, 103.02, 52.75, 46.84, 46.36, 42.57, 34.84, 18.92, 18.66, 15.98. C₃₁H₃₅F₂N₇O, HRMS calculated for m/z [M+H]⁺. 560.2949 (calculated), 560.2955 (found).

### Example 28: N-(1-(3-(1-ethyl-1H-pyrazol-3-yl)-5-(1-(2,2,2-trifluoroethyl)-1H-pyrazol-4-yl)phenyl)cyclopropyl)-2-methyl-5-(4-methylpiperazin-1-yl)benzamide

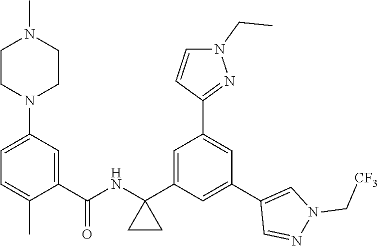 [SMILES: CCn1ccc(-c2cc(-c3cnn(CC(F)(F)F)c3)cc(C3(NC(=O)c4cc(N5CCN(C)CC5)ccc4C)CC3)c2)n1]

(Method A) White solid, 75% yield. ¹H NMR (400 MHz, DMSO-d₆) δ 8.92 (s, 1H), 8.29 (s, 1H), 8.01 (s, 1H), 7.72 (d, J=2.1 Hz, 2H), 7.62 (s, 1H), 7.21 (s, 1H), 7.07 (d, J=8.2 Hz, 1H), 6.95-6.89 (m, 2H), 6.67 (d, J=2.3 Hz, 1H), 5.10 (q, J=9.1 Hz, 3H), 5.03 (s, 2H), 4.10 (q, J=7.3 Hz, 2H), 3.76 (d, J=13.1 Hz, 2H), 3.46 (d, J=12.0 Hz, 2H), 2.89 (t, J=12.3 Hz, 2H), 2.80 (s, 3H), 2.22 (s, 3H), 1.34 (t, J=7.2 Hz, 3H), 1.32-1.28 (m, 2H), 1.25 (q, J=8.0, 6.6 Hz, 2H). ¹³C NMR (101 MHz, DMSO-d₆) δ 170.18, 150.27, 147.70, 145.02, 138.52, 138.08, 134.37, 132.38, 131.70, 131.15, 129.55, 127.22, 123.66, 120.73, 120.68, 119.97, 117.60, 115.34, 102.95, 52.75, 46.83, 46.36, 42.57, 34.84, 18.90, 18.50, 15.99. C₃₂H₃₆F₃N₇O, HRMS calculated for m/z [M+H]⁺: 592.3012 (calculated), 592.3019 (found).

### Example 29: N-(1-(3-(1-ethyl-1H-pyrazol-3-yl)-5-(thiazol-5-yl)phenyl)cyclopropyl)-2-methyl-5-(4-methylpiperazin-1-yl)benzamide

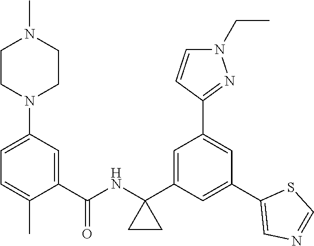 [SMILES: CCn1ccc(-c2cc(-c3cncs3)cc(C3(NC(=O)c4cc(N5CCN(C)CC5)ccc4C)CC3)c2)n1]

(Method A) White solid, 71% yield. ¹H NMR (400 MHz, DMSO-d₆) δ 9.05 (s, 1H), 8.98 (s, 1H), 8.31 (s, 1H), 7.81 (s, 1H), 7.75 (d, J=2.3 Hz, 1H), 7.64 (s, 1H), 7.33 (s, 1H), 7.08 (d, J=8.3 Hz, 1H), 6.96-6.90 (m, 2H), 6.73 (d, J=2.3 Hz, 1H), 4.86 (s, 2H), 4.12 (q, J=7.3 Hz, 2H), 3.77 (d, J=13.1 Hz, 2H), 3.48 (d, J=12.0 Hz, 2H), 2.90 (t, J=12.5 Hz, 2H), 2.81 (s, 3H), 2.21 (s, 3H), 1.38-1.30 (m, 5H), 1.30-1.24 (m, 2H). ¹³C NMR (101 MHz, DMSO-d₆) δ 170.25, 154.00, 149.69, 147.73, 145.63, 140.00, 139.25, 137.95, 134.90, 131.73, 131.40, 131.34, 127.18, 122.55, 121.95, 121.06, 117.70, 115.29, 103.23, 52.75, 46.88, 46.37, 42.57, 34.75, 18.92, 18.61, 15.99. C₃₀H₃₄N₆OS, HRMS calculated for m/z [M+H]⁺: 527.2593 (calculated), 527.2603 (found).

### Example 30: 5-(4-cyclobutylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide

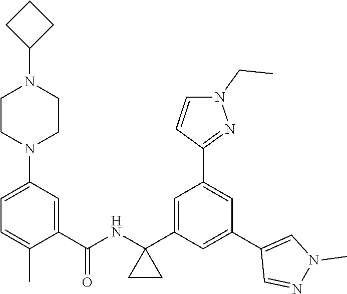 [SMILES: CCn1ccc(-c2cc(-c3cnn(C)c3)cc(C3(NC(=O)c4cc(N5CCN(C6CCC6)CC5)ccc4C)CC3)c2)n1]

(Method A) White solid, 78% yield. ¹H NMR (400 MHz, DMSO-d₆) δ 8.93 (s, 1H), 8.12 (s, 1H), 7.81 (s, 1H), 7.71 (d, J=2.2 Hz, 1H), 7.68 (s, 1H), 7.57 (s, 1H), 7.17 (s, 1H), 7.08 (d, J=8.4 Hz, 1H), 6.92 (dd, J=8.3, 2.7 Hz, 1H), 6.89 (d, J=2.6 Hz, 1H), 6.65 (d, J=2.3 Hz, 1H), 5.02 (s, 2H), 4.10 (q, J=7.2 Hz, 2H), 3.81 (s, 3H), 3.77 (d, J=9.9 Hz, 2H), 3.72-3.64 (m, 1H), 2.88 (d, J=8.9 Hz, 4H), 2.22 (s, 3H), 2.20-2.11 (m, 4H), 1.75-1.63 (m, 2H), 1.34 (t, J=7.3 Hz, 3H), 1.31-1.27 (m, 2H), 1.26-1.20 (m, 2H). ¹³C NMR (101 MHz, DMSO-d₆) δ 170.15, 150.38, 147.70, 144.85, 138.14, 136.48, 134.28, 133.18, 131.71, 131.11, 128.45, 127.26, 122.54, 120.42, 120.19, 119.76, 117.63, 115.36, 102.88, 58.33, 48.20, 46.81, 46.18, 39.09, 34.83, 25.21, 18.91, 18.44, 15.99, 13.65. C₃₄H₄₁N₇O, HRMS calculated for m/z [M+H]⁺: 564.3451 (calculated), 564.3462 (found).

### Example 31: (S)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide

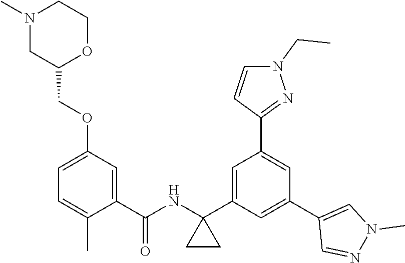

(Method A) White solid, 75% yield. ¹H NMR (400 MHz, DMSO-d₆) δ 9.02 (s, 1H), 8.19 (s, 1H), 7.88 (s, 1H), 7.78 (s, 1H), 7.75 (s, 1H), 7.65 (s, 1H), 7.23 (s, 1H), 7.18 (d, J=8.1 Hz, 1H), 6.95 (d, J=9.5 Hz, 2H), 6.72 (s, 1H), 4.18 (q, J=7.3 Hz, 2H), 4.13-4.02 (m, 4H), 3.88 (s, 3H), 3.76 (t, J=12.4 Hz, 1H), 3.57 (d, J=12.2 Hz, 1H), 3.44 (d, J=12.4 Hz, 1H), 3.07 (q, J=11.5, 10.9 Hz, 2H), 2.87 (s, 3H), 2.30 (s, 3H), 1.42 (t, J=7.2 Hz, 3H), 1.37 (d, J=7.9 Hz, 2H), 1.30 (q, J=7.0, 5.2 Hz, 2H). ¹³C NMR (101 MHz, DMSO-d₆) δ 138.69, 134.21, 133.31, 130.65, 122.54, 122.40, 122.00, 117.85, 116.30, 105.09, 74.46, 70.65, 66.08, 56.09, 54.69, 49.02, 45.52, 42.84, 42.64, 42.43, 42.22, 42.01, 41.80, 41.59, 41.30, 21.06, 20.63, 18.18. C₃₂H₃₈N₆O₃, HRMS calculated for m/z [M+H]⁺: 555.3084 (calculated), 555.3093 (found).

### Example 32: (R)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide

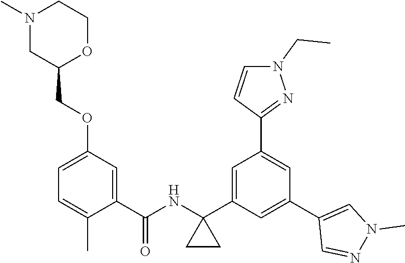

(Method A) White solid, 77% yield. ¹H NMR (400 MHz, DMSO-d₆) δ 8.94 (s, 1H), 8.12 (s, 1H), 7.81 (s, 1H), 7.71 (s, 1H), 7.67 (s, 1H), 7.58 (s, 1H), 7.15 (s, 1H), 7.11 (d, J=8.2 Hz, 1H), 6.88 (d, J=9.6 Hz, 2H), 6.65 (d, J=2.2 Hz, 1H), 4.10 (q, J=7.3 Hz, 2H), 4.00 (q, J=11.1, 7.9 Hz, 4H), 3.81 (s, 3H), 3.68 (t, J=12.4 Hz, 1H), 3.50 (d, J=12.2 Hz, 1H), 3.36 (d, J=12.5 Hz, 1H), 2.99 (q, J=10.7, 10.2 Hz, 2H), 2.79 (s, 3H), 2.23 (s, 3H), 1.34 (t, J=7.2 Hz, 3H), 1.28 (d, J=5.0 Hz, 2H), 1.23 (d, J=4.7 Hz, 2H). ¹³C NMR (101 MHz, DMSO-d₆) δ 156.21, 150.39, 144.78, 138.57, 136.48, 134.29, 133.22, 132.00, 131.10, 128.44, 128.10, 122.56, 120.33, 120.19, 119.79, 115.64, 114.09, 102.88, 72.25, 68.44, 63.87, 53.88, 52.47, 46.81, 43.31, 39.09, 34.81, 18.85, 18.42, 15.97. C₃₂H₃₈N₆O₃, HRMS calculated for m/z [M+H]⁺. 555.3084 (calculated), 555.3092 (found).

### Example 33: (R)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide

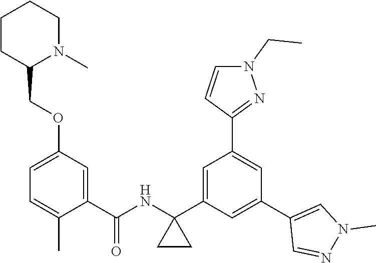

(Method A) White solid, 75% yield. ¹H NMR (400 MHz, DMSO-d₆) δ 8.95 (s, 1H), 8.12 (s, 1H), 7.80 (s, 1H), 7.71 (s, 1H), 7.68 (s, 1H), 7.57 (s, 1H), 7.16 (d, J=9.7 Hz, 1H), 7.12 (d, J=8.5 Hz, 1H), 6.99-6.89 (m, 2H), 6.65 (s, 1H), 4.81 (d, J=21.1 Hz, 1H), 4.10 (q, J=7.2 Hz, 2H), 3.81 (s, 3H), 3.50 (d, J=7.0 Hz, 1H), 3.41-3.30 (m, 1H), 3.26-3.11 (m, 1H), 2.81 (s, 2H), 2.73 (dd, J=26.0, 4.6 Hz, 1H), 2.24 (s, 3H), 2.01-1.50 (m, 6H), 1.35 (t, J=7.2 Hz, 3H), 1.29 (d, J=4.7 Hz, 2H), 1.22 (d, J=4.7 Hz, 2H). ¹³C NMR (101 MHz, DMSO-d₆) δ 169.67, 150.39, 144.74, 138.82, 136.48, 134.31, 133.22, 132.09, 131.10, 128.45, 122.55, 120.49, 120.26, 119.84, 118.00, 115.87, 115.08, 102.89, 58.89, 57.48, 46.81, 45.89, 41.17, 39.09, 34.85, 30.67, 24.22, 23.18, 21.11, 18.89, 18.35, 15.98. C₃₃H₄₀N₆O₂, HRMS calculated for m/z [M+H]⁺: 553.3291 (calculated), 553.3302 (found).

### Example 34: (S)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide

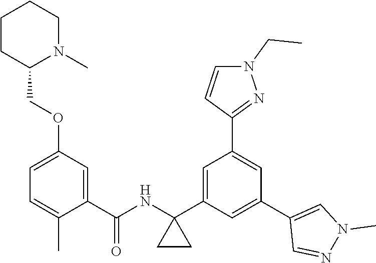 [SMILES: CCn1ccc(-c2cc(-c3cnn(C)c3)cc(C3(NC(=O)c4cc(OC[C@@H]5CCCCN5C)ccc4C)CC3)c2)n1]

(Method A) White solid, 75% yield. ¹H NMR (400 MHz, DMSO-d₆) δ 8.95 (s, 1H), 8.12 (s, 1H), 7.80 (s, 1H), 7.71 (s, 1H), 7.68 (s, 1H), 7.57 (s, 1H), 7.16 (d, J=9.8 Hz, 1H), 7.12 (d, J=8.5 Hz, 1H), 6.99-6.89 (m, 2H), 6.65 (s, 1H), 4.81 (d, J=19.8 Hz, 1H), 4.10 (q, J=7.1 Hz, 2H), 3.81 (s, 3H), 3.50 (d, J=7.1 Hz, 1H), 3.37 (d, J=14.5 Hz, 1H), 3.24-2.98 (m, 1H), 2.82 (s, 2H), 2.73 (dd, J=26.0, 4.6 Hz, 1H), 2.24 (s, 3H), 1.97-1.47 (m, 6H), 1.35 (t, J=7.2 Hz, 3H), 1.29 (d, J=4.7 Hz, 2H), 1.22 (d, J=4.6 Hz, 2H). ¹³C NMR (101 MHz, DMSO-d₆) δ 169.67, 150.39, 144.73, 136.48, 134.31, 133.22, 132.09, 131.10, 128.45, 122.54, 120.49, 120.29, 119.84, 118.09, 115.86, 115.16, 102.89, 72.15, 63.46, 58.88, 57.48, 46.81, 45.89, 41.17, 39.09, 34.85, 30.68, 24.22, 21.11, 18.89, 18.34, 15.98. C₃₃H₄₀N₆O₂, HRMS calculated for m/z [M+H]⁺: 553.3291 (calculated), 553.3301 (found).

### Example 35: N-(1-(3-(1-ethyl-1H-pyrazol-3-yl)-5-(1-methyl-1H-pyrazol-4-yl)phenyl)cyclopropyl)-2-methyl-5-(2-thiomorpholinoethoxy)benzamide

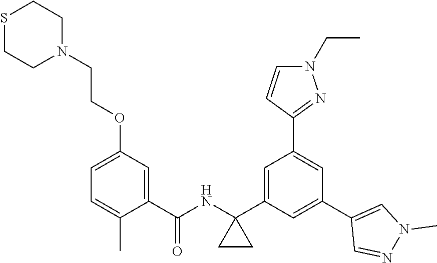 [SMILES: CCn1ccc(-c2cc(-c3cnn(C)c3)cc(C3(NC(=O)c4cc(OCCN5CCSCC5)ccc4C)CC3)c2)n1]

(Method A) White solid, 75% yield. ¹H NMR (400 MHz, DMSO-d₆) δ 8.95 (s, 1H), 8.12 (s, 1H), 7.80 (s, 1H), 7.71 (s, 1H), 7.68 (s, 1H), 7.57 (s, 1H), 7.20-7.11 (m, 2H), 6.91 (d, J=6.4 Hz, 2H), 6.64 (s, 1H), 4.30 (t, J=5.0 Hz, 2H), 4.10 (q, J=7.3 Hz, 2H), 3.81 (s, 3H), 3.71 (s, 2H), 3.51 (t, J=5.0 Hz, 2H), 3.23 (s, 2H), 2.89 (d, J=36.4 Hz, 4H), 2.24 (s, 3H), 1.35 (t, J=7.2 Hz, 3H), 1.29 (d, J=5.0 Hz, 2H), 1.23 (d, J=5.1 Hz, 2H). ¹³C NMR (101 MHz, DMSO-d₆) δ 169.68, 155.66, 150.39, 144.74, 138.66, 136.47, 134.32, 133.21, 132.02, 131.10, 128.45, 128.43, 122.55, 120.53, 120.27, 119.84, 115.91, 114.20, 102.89, 62.61, 55.76, 53.93, 46.81, 39.10, 34.87, 24.41, 18.90, 18.33, 15.97. C₃₂H₃₈N₆O₂S, HRMS calculated for m/z [M+H]⁺: 571.2855 (calculated), 571.2861 (found).

### Example 36: (S)-N-(1-(3,5-bis(1-ethyl-1H-pyrazol-3-yl)phenyl)cyclopropyl)-2-methyl-5-((1-methylazetidin-2-yl)methoxy)benzamide

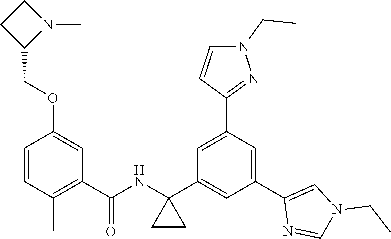

(Method A) White solid, 75% yield. ¹H NMR (400 MHz, DMSO-d₆) δ 9.00 (s, 1H), 7.92 (s, 1H), 7.72 (d, J=2.2 Hz, 2H), 7.54 (d, J=2.6 Hz, 2H), 7.13 (d, J=7.2 Hz, 1H), 6.97-6.84 (m, 2H), 6.64 (d, J=2.3 Hz, 2H), 5.11 (s, 1H), 4.60 (s, 1H), 4.26 (d, J=6.1 Hz, 1H), 4.11 (q, J=7.2 Hz, 4H), 4.04-3.60 (m, 2H), 3.35-3.04 (m, 1H), 2.89-2.78 (m, 3H), 2.39-2.27 (m, 1H), 2.25 (s, 3H), 1.35 (t, J=7.3 Hz, 6H), 1.26 (d, J=14.9 Hz, 4H). ¹³C NMR (101 MHz, DMSO-d₆) δ 169.65, 155.77, 154.38, 150.36, 144.47, 138.67, 134.17, 132.01, 131.17, 128.39, 121.14, 119.83, 115.82, 115.32, 114.20, 102.86, 68.22, 67.08, 60.19, 53.04, 46.82, 41.43, 34.88, 34.84, 18.94, 18.92, 18.26, 16.03. C₃₂H₃₈N₆O₂, HRMS calculated for m/z [M+H]⁺: 539.3134 (calculated), 539.3141 (found).

### Example 37: (S)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide

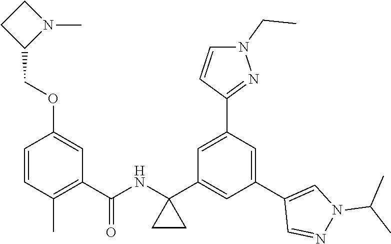

(Method A) White solid, 75% yield. ¹H NMR (400 MHz, DMSO-d₆) δ 8.96 (s, 1H), 8.20 (s, 1H), 7.81 (s, 1H), 7.72 (d, J=2.2 Hz, 1H), 7.70 (s, 1H), 7.58 (s, 1H), 7.18 (d, J=8.4 Hz, 1H), 7.13 (d, J=7.6 Hz, 1H), 6.94-6.84 (m, 2H), 6.66 (d, J=2.3 Hz, 1H), 4.45 (p, J=6.7 Hz, 1H), 4.26 (d, J=5.5 Hz, 1H), 4.10 (q, J=7.3 Hz, 2H), 4.04-3.62 (m, 2H), 3.34-3.03 (m, 1H), 2.90-2.79 (m, 3H), 2.42 (d, J=3.8 Hz, 1H), 2.34 (t, J=8.9 Hz, 1H), 2.25 (d, J=1.9 Hz, 3H), 2.20-1.95 (m, 1H), 1.39 (d, J=6.6 Hz, 6H), 1.35 (t, J=7.3 Hz, 3H), 1.29 (d, J=4.0 Hz, 2H), 1.23 (d, J=4.8 Hz, 2H). ¹³C NMR (101 MHz, DMSO-d₆) δ 169.66, 169.61, 155.79, 154.39, 150.43, 144.69, 138.79, 138.71, 135.97, 134.25, 133.42, 131.09, 125.27, 122.05, 120.45, 120.20, 119.81, 115.82, 115.31, 114.18, 102.91, 76.02, 68.23, 67.10, 60.20, 53.61, 46.80, 41.44, 34.88, 34.86, 23.16, 18.92, 18.90, 18.41, 16.00. C₃₃H₄₀N₆O₂, HRMS calculated for m/z [M+H]⁺: 553.3291 (calculated), 553.3299i (found).

### Example 38: N-(1-(3-(1-ethyl-1H-pyrazol-3-yl)-5-(1-(2-fluoroethyl)-1H-pyrazol-4-yl)phenyl)cyclopropyl)-2-methyl-5-(4-methylpiperazin-1-yl)benzamide

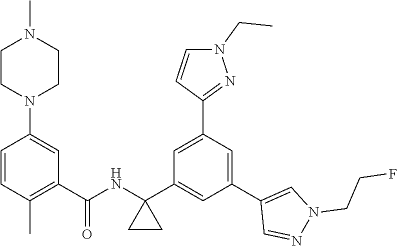 [SMILES: CCn1ccc(-c2cc(-c3cnn(CCF)c3)cc(C3(NC(=O)c4cc(N5CCN(C)CC5)ccc4C)CC3)c2)n1]

(Method A) White solid, 72% yield. ¹H NMR (400 MHz, DMSO-d₆) δ 8.91 (s, 1H), 8.21 (s, 1H), 7.89 (s, 1H), 7.72 (d, J=2.2 Hz, 1H), 7.70 (s, 1H), 7.58 (s, 1H), 7.19 (s, 1H), 7.07 (d, J=8.2 Hz, 1H), 6.92 (d, J=8.5 Hz, 2H), 6.66 (d, J=2.3 Hz, 1H), 4.80 (t, J=4.7 Hz, 1H), 4.69 (t, J=4.7 Hz, 1H), 4.43 (t, J=4.8 Hz, 1H), 4.36 (t, J=4.7 Hz, 1H), 4.10 (q, J=7.2 Hz, 2H), 3.76 (d, J=13.2 Hz, 2H), 3.46 (d, J=12.0 Hz, 2H), 3.08 (t, J=11.7 Hz, 2H), 2.89 (t, J=12.6 Hz, 2H), 2.80 (s, 3H), 2.22 (s, 3H), 1.34 (t, J=7.3 Hz, 3H), 1.29 (d, J=4.1 Hz, 2H), 1.26-1.21 (m, 2H). 13C NMR (101 MHz, DMSO-d₆) δ 170.15, 150.38, 147.70, 144.90, 138.12, 137.08, 134.30, 133.03, 131.70, 131.11, 128.34, 127.21, 122.68, 120.47, 120.33, 119.80, 117.59, 115.33, 102.90, 83.37, 81.70, 52.75, 52.54, 52.35, 46.82, 46.36, 42.57, 34.84, 18.91, 18.49, 16.00. C₃₂H₃₈FN₇O, HRMS calculated for m/z [M+H]⁺: 556.3200 (calculated), 556.3208 (found).

### Example 39: N-(1-(3-(1-ethyl-1H-pyrazol-3-yl)-5-(4-methylthiazol-5-yl)phenyl)cyclopropyl)-2-methyl-5-(4-methylpiperazin-1-yl)benzamide

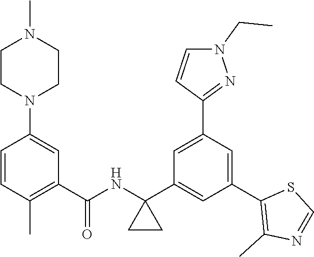 [SMILES: CCn1ccc(-c2cc(-c3scnc3C)cc(C3(NC(=O)c4cc(N5CCN(C)CC5)ccc4C)CC3)c2)n1]

(Method A) White solid, 79% yield. ¹H NMR (400 MHz, DMSO-d₆) δ 8.96 (s, 2H), 7.73 (s, 1H), 7.61 (s, 2H), 7.20 (s, 1H), 7.07 (d, J=8.4 Hz, 1H), 6.96-6.89 (m, 2H), 6.68 (d, J=2.3 Hz, 1H), 4.11 (q, J=7.3 Hz, 2H), 3.75 (d, J=13.1 Hz, 2H), 3.48 (d, J=12.0 Hz, 2H), 3.09 (t, J=11.5 Hz, 2H), 2.89 (t, J=12.4 Hz, 2H), 2.81 (s, 3H), 2.42 (s, 3H), 2.20 (s, 3H), 1.34 (t, J=7.2 Hz, 3H), 1.30 (d, J=5.0 Hz, 2H), 1.27 (d, J=4.3 Hz, 2H). ¹³C NMR (101 MHz, DMSO-d₆) δ 170.20, 152.05, 149.75, 148.51, 147.73, 145.20, 137.88, 134.59, 132.07, 131.85, 131.75, 131.36, 127.25, 124.94, 123.44, 121.43, 117.75, 115.29, 103.13, 52.75, 46.87, 46.38, 42.57, 34.77, 18.91, 18.66, 16.48, 15.96. C₃₁H₃₆N₆OS, HRMS calculated for m/z [M+H]⁺: 541.2750 (calculated), 541.2758 (found).

### Example 40: N-(1-(3-(1-ethyl-1H-pyrazol-3-yl)-5-(thiazol-2-yl)phenyl)cyclopropyl)-2-methyl-5-(4-methylpiperazin-1-yl)benzamide

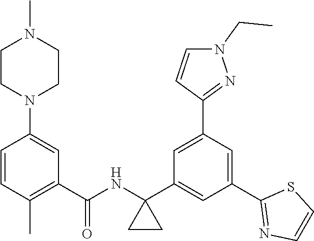 [SMILES: CCn1ccc(-c2cc(-c3nccs3)cc(C3(NC(=O)c4cc(N5CCN(C)CC5)ccc4C)CC3)c2)n1]

(Method A) White solid, 75% yield. ¹H NMR (400 MHz, DMSO-d₆) δ 9.02 (s, 1H), 8.10 (s, 1H), 7.88 (d, J=3.3 Hz, 1H), 7.75 (t, J=2.8 Hz, 3H), 7.62 (s, 1H), 7.08 (d, J=9.1 Hz, 1H), 6.94 (q, J=3.4, 2.9 Hz, 2H), 6.72 (d, J=2.2 Hz, 1H), 4.13 (q, J=7.3 Hz, 2H), 3.77 (d, J=13.2 Hz, 2H), 3.48 (d, J=12.1 Hz, 2H), 3.18-3.03 (m, 2H), 2.89 (d, J=12.5 Hz, 2H), 2.81 (s, 3H), 2.21 (s, 3H), 1.36 (t, J=7.3 Hz, 3H), 1.33 (t, J=2.4 Hz, 2H), 1.30-1.27 (m, 2H). ¹³C NMR (101 MHz, DMSO-d₆) δ 170.25, 167.73, 149.56, 147.73, 145.62, 144.27, 137.96, 134.95, 133.76, 131.72, 131.45, 127.19, 123.24, 122.28, 120.92, 120.56, 117.71, 115.26, 103.14, 52.75, 46.91, 46.37, 42.58, 34.72, 18.88, 18.58, 16.00. C₃₀H₃₄N₆OS, HRMS calculated for m/z [M+H]⁺: 527.2593 (calculated), 527.2601 (found).

### Example 41: N-(1-(3-(1-ethyl-1H-pyrazol-3-yl)-5-(5-methylthiazol-2-yl)phenyl)cyclopropyl)-2-methyl-5-(4-methylpiperazin-1-yl)benzamide

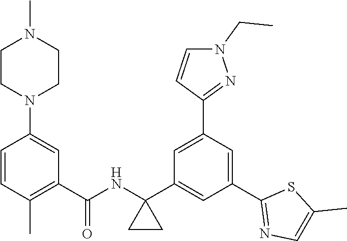 [SMILES: CCn1ccc(-c2cc(-c3ncc(C)s3)cc(C3(NC(=O)c4cc(N5CCN(C)CC5)ccc4C)CC3)c2)n1]

(Method A) White solid, 75% yield. ¹H NMR (400 MHz, DMSO-d₆) δ 9.01 (s, 1H), 8.02 (s, 1H), 7.75 (d, J=2.3 Hz, 1H), 7.67 (s, 1H), 7.59 (s, 1H), 7.55 (s, 1H), 7.08 (d, J=9.1 Hz, 1H), 6.94 (dd, J=5.9, 2.8 Hz, 2H), 6.70 (d, J=2.3 Hz, 1H), 4.13 (q, J=7.3 Hz, 2H), 3.77 (d, J=13.3 Hz, 2H), 3.48 (d, J=12.1 Hz, 2H), 3.18-3.04 (m, 2H), 2.91 (t, J=12.5 Hz, 2H), 2.81 (s, 3H), 2.44 (s, 3H), 2.21 (s, 3H), 1.35 (t, J=7.3 Hz, 3H), 1.30 (d, J=3.4 Hz, 2H), 1.28 (d, J=3.0 Hz, 2H). ¹³C NMR (101 MHz, DMSO-d₆) δ 170.23, 165.92, 149.60, 147.70, 145.52, 142.14, 137.95, 134.87, 134.67, 134.00, 131.73, 131.43, 127.19, 122.95, 121.83, 120.19, 117.68, 115.25, 103.07, 52.74, 46.90, 46.34, 42.58, 34.70, 18.88, 18.58, 15.99, 12.12. C₃₁H₃₆N₆OS, HRMS calculated for m/z [M+H]⁺: 541.2750 (calculated), 541.2759 (found).

### Example 42: 5-(2-(dimethylamino)ethoxy)-N-(1-(3-(1-ethyl-1H-pyrazol-3-yl)-5-(thiazol-5-yl)phenyl)cyclopropyl)-2-methylbenzamide

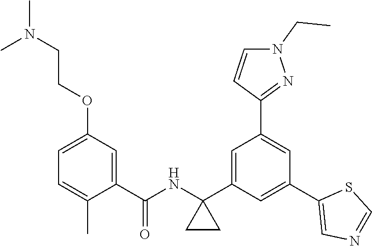 [SMILES: CCn1ccc(-c2cc(-c3cncs3)cc(C3(NC(=O)c4cc(OCCN(C)C)ccc4C)CC3)c2)n1]

(Method A) White solid, 75% yield. ¹H NMR (400 MHz, DMSO-d₆) δ 9.04 (d, J=5.4 Hz, 2H), 8.31 (s, 1H), 7.81 (s, 1H), 7.75 (d, J=2.2 Hz, 1H), 7.65 (s, 1H), 7.33 (s, 1H), 7.14 (d, J=9.2 Hz, 1H), 6.93 (q, J=5.3 Hz, 2H), 6.73 (d, J=2.2 Hz, 1H), 4.26 (t, J=5.0 Hz, 2H), 4.12 (q, J=7.2 Hz, 2H), 3.46 (q, J=4.2 Hz, 1H), 3.12 (s, 1H), 2.80 (d, J=2.7 Hz, 6H), 2.24 (s, 3H), 1.35 (t, J=7.1 Hz, 5H), 1.26 (d, J=4.9 Hz, 2H). ¹³C NMR (101 MHz, DMSO-d₆) δ 169.79, 155.70, 154.02, 149.68, 145.50, 140.02, 139.23, 138.45, 134.91, 132.06, 131.44, 131.35, 128.40, 122.57, 122.08, 121.16, 116.02, 114.13, 103.22, 62.89, 55.88, 46.88, 43.26, 34.79, 18.92, 18.49, 15.98. C₂₉H₃₃N₅O₂S, HRMS calculated for m/z [M+H]⁺: 516.2433 (calculated), 516.2441 (found).

### Example 43: 5-(2-(dimethylamino)ethoxy)-N-(1-(3-(1-ethyl-1H-pyrazol-3-yl)-5-(thiazol-2-yl)phenyl)cyclopropyl)-2-methylbenzamide

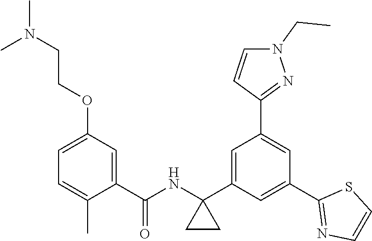 [SMILES: CCn1ccc(-c2cc(-c3nccs3)cc(C3(NC(=O)c4cc(OCCN(C)C)ccc4C)CC3)c2)n1]

(Method A) White solid, 69% yield. ¹H NMR (400 MHz, DMSO-d₆) δ 9.08 (s, 1H), 8.10 (s, 1H), 7.88 (d, J=3.3 Hz, 1H), 7.75 (q, J=4.9, 3.9 Hz, 3H), 7.66 (s, 1H), 7.14 (d, J=8.9 Hz, 1H), 6.93 (d, J=7.1 Hz, 2H), 6.72 (d, J=2.3 Hz, 1H), 4.26 (t, J=5.0 Hz, 2H), 4.14 (q, J=7.3 Hz, 2H), 3.46 (d, J=5.5 Hz, 2H), 2.80 (s, 6H), 2.24 (s, 3H), 1.36 (t, J=7.3 Hz, 3H), 1.33 (d, J=4.6 Hz, 2H), 1.30-1.25 (m, 2H). ¹³C NMR (101 MHz, DMSO-d₆) δ 169.79, 167.70, 155.69, 149.55, 145.47, 144.28, 138.41, 134.93, 133.81, 132.07, 131.47, 128.44, 123.45, 122.33, 120.95, 120.63, 116.05, 114.11, 103.13, 62.89, 55.88, 46.91, 43.26, 34.79, 18.92, 18.44, 16.00.

C₂₉H₃₃N₅O₂S, HRMS calculated for m/z [M+H]⁺: 516.2433 (calculated), 516.2442 (found).

### Example 44: N-(1-(3-(1-(2-cyanoethyl)-1H-pyrazol-4-yl)-5-(1-ethyl-1H-pyrazol-3-yl)phenyl)cyclopropyl)-2-methyl-5-(4-methylpiperazin-1-yl)benzamide

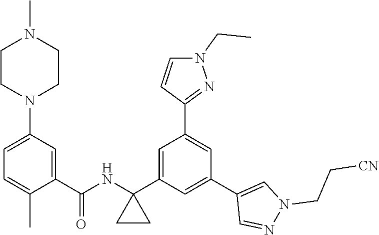 [SMILES: CCn1ccc(-c2cc(-c3cnn(CCC#N)c3)cc(C3(NC(=O)c4cc(N5CCN(C)CC5)ccc4C)CC3)c2)n1]

(Method A) White solid, 79% yield. ¹H NMR (400 MHz, DMSO-d₆) δ 8.92 (s, 1H), 8.26 (s, 1H), 7.92 (s, 1H), 7.72 (d, J=2.3 Hz, 1H), 7.70 (s, 1H), 7.59 (s, 1H), 7.19 (s, 1H), 7.07 (d, J=8.2 Hz, 1H), 6.94-6.89 (m, 2H), 6.65 (d, J=2.3 Hz, 1H), 4.36 (t, J=6.4 Hz, 2H), 4.10 (q, J=7.2 Hz, 2H), 3.76 (d, J=13.1 Hz, 2H), 3.46 (d, J=12.0 Hz, 2H), 3.09 (s, 2H), 3.05 (t, J=6.4 Hz, 2H), 2.89 (t, J=12.6 Hz, 2H), 2.80 (s, 3H), 2.22 (s, 3H), 1.34 (t, J=7.2 Hz, 3H), 1.29 (q, J=3.5, 1.9 Hz, 2H), 1.26-1.22 (m, 2H). ¹³C NMR (101 MHz, DMSO-d₆) δ 170.15, 150.34, 147.70, 144.95, 138.11, 137.44, 134.32, 132.90, 131.70, 131.14, 128.15, 127.21, 122.71, 120.49, 119.81, 119.02, 117.59, 115.33, 102.87, 52.75, 47.46, 46.83, 46.36, 42.57, 34.84, 19.01, 18.93, 18.48, 16.00. C₃₃H₃₈N₈O, HRMS calculated for m/z [M+H]⁺: 563.3247 (calculated), 563.3255 (found).

### Example 45: N-(1-(3-(1-ethyl-1H-pyrazol-3-yl)-5-(1-(2-hydroxyethyl)-1H-pyrazol-4-yl)phenyl)cyclopropyl)-2-methyl-5-(4-methylpiperazin-1-yl)benzamide

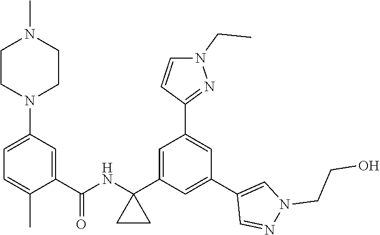 [SMILES: CCn1ccc(-c2cc(-c3cnn(CCO)c3)cc(C3(NC(=O)c4cc(N5CCN(C)CC5)ccc4C)CC3)c2)n1]

(Method A) White solid, 78% yield. ¹H NMR (400 MHz, DMSO-d₆) δ 8.95 (s, 1H), 8.19 (s, 1H), 7.90 (s, 1H), 7.79 (d, J=2.2 Hz, 1H), 7.75 (s, 1H), 7.63 (s, 1H), 7.22 (s, 1H), 7.09 (d, J=9.2 Hz, 1H), 6.95-6.91 (m, 2H), 6.72 (d, J=2.3 Hz, 1H), 4.94 (s, 1H), 4.17 (dd, J=6.8, 3.9 Hz, 4H), 3.78 (q, J=5.1 Hz, 2H), 3.30 (s, 2H), 3.14 (t, J=5.2 Hz, 4H), 2.25 (s, 7H), 1.42 (t, J=7.2 Hz, 3H), 1.35 (d, J=5.0 Hz, 2H), 1.30 (d, J=4.6 Hz, 2H). ¹³C NMR (101 MHz, DMSO-d₆) δ 170.42, 150.44, 149.21, 144.95, 137.98, 136.49, 134.25, 133.27, 131.49, 131.07, 128.14, 125.80, 122.20, 120.24, 120.03, 119.69, 117.07, 114.65, 102.87, 60.51, 54.96, 54.74, 48.76, 46.81, 46.07, 34.73, 18.82, 18.56, 16.02. C₃₂H₃₉N₇O₂, HRMS calculated for m/z [M+H]⁺: 554.3243 (calculated), 554.3250 (found).

### Example 46: 2-methyl-N-(1-(3-(1-methyl-1H-pyrazol-3-yl)-5-(1-methyl-1H-pyrazol-4-yl)phenyl)cyclopropyl)-5-(4-methylpiperazin-1-yl)benzamide

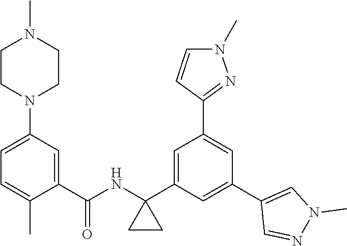 [SMILES: Cc1ccc(N2CCN(C)CC2)cc1C(=O)NC1(c2cc(-c3cnn(C)c3)cc(-c3ccn(C)n3)c2)CC1]

(Method A) White solid, 70% yield. ¹H NMR (400 MHz, DMSO-d₆) δ 8.90 (s, 1H), 8.11 (s, 1H), 7.80 (s, 1H), 7.70-7.65 (m, 2H), 7.54 (s, 1H), 7.18 (s, 1H), 7.07 (d, J=8.1 Hz, 1H), 6.92 (d, J=8.6 Hz, 2H), 6.64 (d, J=2.2 Hz, 1H), 3.81 (d, J=2.4 Hz, 6H), 3.79-3.74 (m, 2H), 3.47 (d, J=12.0 Hz, 2H), 3.15-3.04 (m, 2H), 2.89 (t, J=12.5 Hz, 2H), 2.80 (s, 3H), 2.20 (s, 3H), 1.28 (d, J=4.9 Hz, 2H), 1.23 (t, J=6.3 Hz, 2H). ¹³C NMR (101 MHz, DMSO-d₆) δ 170.13, 150.57, 147.70, 144.89, 138.12, 136.46, 134.17, 133.19, 132.67, 131.70, 128.43, 127.22, 122.55, 120.51, 120.12, 119.71, 117.63, 115.30, 103.02, 52.77, 46.38, 42.58, 39.10, 34.81, 18.87, 18.44. C₃₀H₃₅N₇O, HRMS calculated for m/z [M+H]⁺: 510.2981 (calculated), 510.2989 (found).

### Example 47: 5-(2-(diethylamino)ethoxy)-N-(1-(3-(1-ethyl-1H-pyrazol-3-yl)-5-(1-methyl-1H-pyrazol-4-yl)phenyl)cyclopropyl)-2-methylbenzamide

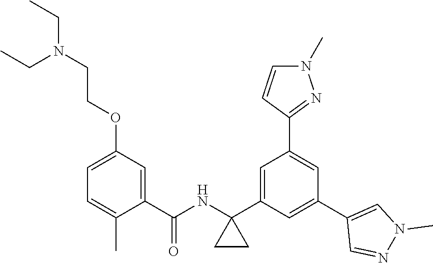 [SMILES: CCN(CC)CCOc1ccc(C)c(C(=O)NC2(c3cc(-c4cnn(C)c4)cc(-c4ccn(C)n4)c3)CC2)c1]

(Method A) White solid, 71% yield. ¹H NMR (400 MHz, DMSO-d₆) δ 9.05 (s, 1H), 8.20 (s, 1H), 7.89 (s, 1H), 7.79 (d, J=2.3 Hz, 1H), 7.76 (s, 1H), 7.65 (s, 1H), 7.25 (s, 1H), 7.21 (d, J=9.1 Hz, 1H), 6.99 (d, J=7.1 Hz, 2H), 6.73 (d, J=2.3 Hz, 1H), 4.33 (t, J=5.0 Hz, 2H), 4.18 (q, J=7.2 Hz, 2H), 3.89 (s, 3H), 3.53 (q, J=4.9 Hz, 2H), 3.24 (dq, J=14.0, 6.9 Hz, 4H), 2.32 (s, 3H), 1.42 (t, J=7.2 Hz, 3H), 1.37 (t, J=3.2 Hz, 2H), 1.33-1.29 (m, 2H), 1.24 (t, J=7.2 Hz, 6H). 13C NMR (101 MHz, DMSO-d₆) δ 169.68, 155.70, 150.38, 144.74, 138.65, 136.48, 134.30, 133.21, 132.04, 131.12, 128.46, 128.38, 122.53, 120.45, 120.22, 119.81, 115.89, 114.04, 102.88, 62.92, 50.54, 47.71, 46.81, 39.10, 34.85, 18.91, 18.37, 15.99, 9.01. C₃₂H₄₀N₆O₂, HRMS calculated for m/z [M+H]⁺: 541.3291 (calculated), 541.3299 (found).

### Example 48: 5-(3-(dimethylamino)azetidin-1-yl)-N-(1-(3-(1-isopropyl-1H-pyrazol-4-yl)-5-(1-methyl-1H-pyrazol-4-yl)phenyl)cyclopropyl)-2-methylbenzamide

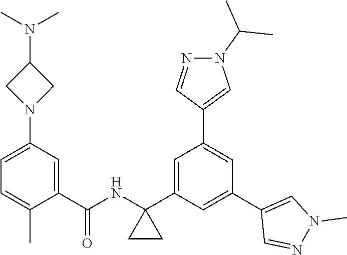 [SMILES: Cc1ccc(N2CC(N(C)C)C2)cc1C(=O)NC1(c2cc(-c3cnn(C)c3)cc(-c3cnn(C(C)C)c3)c2)CC1]

(Method A) White solid, 74% yield. ¹H NMR (400 MHz, DMSO-d₆) δ 8.85 (s, 1H), 8.17 (s, 1H), 8.08 (s, 1H), 7.79 (s, 2H), 7.55 (s, 1H), 7.17 (s, 2H), 7.04 (d, J=7.9 Hz, 1H), 6.44 (d, J=7.8 Hz, 2H), 4.45 (q, J=6.6 Hz, 1H), 4.42-4.33 (m, 1H), 4.04 (t, J=7.9 Hz, 2H), 3.87 (dd, J=8.8, 5.2 Hz, 2H), 3.81 (s, 3H), 2.75 (s, 6H), 2.18 (s, 3H), 1.39 (d, J=6.6 Hz, 6H), 1.34 (d, J=6.6 Hz, 3H), 1.30 (t, J=3.4 Hz, 2H), 1.19 (q, J=5.6, 4.9 Hz, 2H). ¹³C NMR (101 MHz, DMSO-d₆) δ 170.17, 149.02, 145.07, 138.15, 136.45, 135.92, 135.86, 133.48, 133.23, 131.47, 128.32, 125.16, 124.78, 124.02, 122.65, 122.09, 119.73, 119.55, 113.84, 113.33, 111.03, 55.74, 54.49, 53.61, 53.28, 39.13, 34.79, 23.16, 18.85, 18.42. C₃₂H₃₉N₇O, HRMS calculated for m/z [M+H]⁺: 538.3294 (calculated), 538.3302 (found).

### Example 49: N-(1-(3-(1-ethyl-1H-pyrazol-3-yl)-5-(1-ethyl-1H-pyrazol-4-yl)phenyl)cyclopropyl)-2-methyl-5-(4-methylpiperazin-1-yl)benzamide

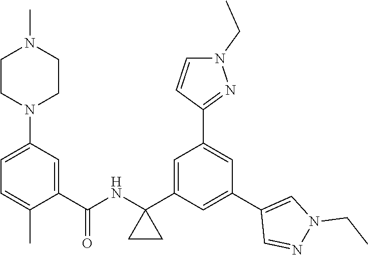 [SMILES: CCn1cc(-c2cc(-c3ccn(CC)n3)cc(C3(NC(=O)c4cc(N5CCN(C)CC5)ccc4C)CC3)c2)cn1]

(Method A) White solid, 85% yield. ¹H NMR (400 MHz, DMSO-d₆) δ 8.91 (s, 1H), 8.17 (s, 1H), 7.82 (s, 1H), 7.72 (d, J=2.3 Hz, 1H), 7.69 (s, 1H), 7.57 (s, 1H), 7.17 (s, 1H), 7.07 (d, J=8.2 Hz, 1H), 6.95-6.89 (m, 2H), 6.65 (d, J=2.3 Hz, 1H), 4.09 (q, J=6.6, 5.8 Hz, 4H), 3.76 (d, J=13.1 Hz, 2H), 3.46 (d, J=12.0 Hz, 2H), 3.15-3.03 (m, 2H), 2.89 (t, J=12.4 Hz, 2H), 2.80 (s, 3H), 2.22 (s, 3H), 1.35 (td, J=7.3, 3.7 Hz, 6H), 1.32-1.27 (m, 2H), 1.26-1.21 (m, 2H). ¹³C NMR (101 MHz, DMSO-d₆) δ 170.14, 150.42, 147.70, 144.84, 138.12, 136.31, 134.24, 133.29, 131.70, 131.10, 127.21, 126.96, 122.32, 120.36, 120.19, 119.74, 117.59, 115.34, 102.88, 52.75, 46.85, 46.81, 46.36, 42.57, 34.83, 18.92, 18.50, 16.00, 15.88. C₃₂H₃₉N₇O, HRMS calculated for m/z [M+H]⁺: 538.3294 (calculated), 538.3301 (found).

### Example 50: N-(1-(3-(1-ethyl-1H-pyrazol-3-yl)-5-(1-(methoxymethyl)-1H-pyrazol-4-yl)phenyl)cyclopropyl)-2-methyl-5-(4-methylpiperazin-1-yl)benzamide

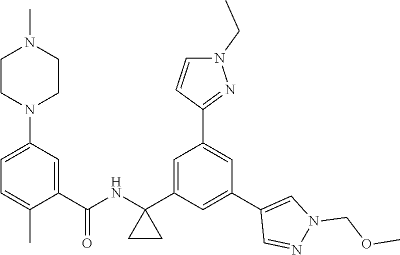 [SMILES: CCn1ccc(-c2cc(-c3cnn(COC)c3)cc(C3(NC(=O)c4cc(N5CCN(C)CC5)ccc4C)CC3)c2)n1]

(Method A) White solid, 81% yield. ¹H NMR (400 MHz, DMSO-d₆) δ 8.92 (s, 1H), 8.35 (s, 1H), 7.96 (s, 1H), 7.73 (s, 1H), 7.72 (d, J=2.2 Hz, 1H), 7.61 (t, J=1.7 Hz, 1H), 7.21 (s, 1H), 7.07 (d, J=8.1 Hz, 1H), 6.92 (d, J=8.2 Hz, 2H), 6.67 (d, J=2.3 Hz, 1H), 5.34 (s, 2H), 4.10 (q, J=7.2 Hz, 2H), 3.76 (d, J=13.1 Hz, 2H), 3.47 (d, J=12.0 Hz, 2H), 3.21 (s, 3H), 3.15-3.04 (m, 2H), 2.89 (t, J=12.7 Hz, 2H), 2.80 (s, 3H), 2.22 (s, 3H), 1.34 (t, J=7.2 Hz, 3H), 1.30 (d, J=4.1 Hz, 2H), 1.27-1.21 (m, 2H). ¹³C NMR (101 MHz, DMSO-d₆) δ 170.16, 150.35, 147.70, 144.95, 138.11, 137.88, 134.31, 132.78, 131.70, 131.13, 128.58, 127.22, 123.40, 120.55, 119.96, 117.59, 115.34, 102.93, 81.78, 56.60, 52.75, 46.82, 46.36, 42.57, 34.84, 18.92, 18.51, 16.00. C₃₂H₃₉N₇O₂, HRMS calculated for m/z [M+H]⁺: 554.3243 (calculated), 554.3250 (found).

### Example 51: (R)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide

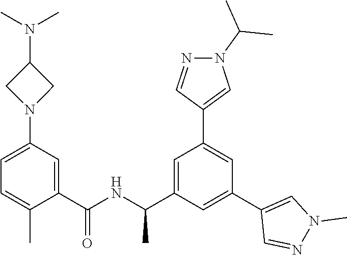 [SMILES: Cc1ccc(N2CC(N(C)C)C2)cc1C(=O)N[C@H](C)c1cc(-c2cnn(C)c2)cc(-c2cnn(C(C)C)c2)c1]

(Method B) White solid, 83% yield. ¹H NMR (400 MHz, DMSO-d₆) δ 8.56 (d, J=8.3 Hz, 1H), 8.18 (s, 1H), 8.09 (s, 1H), 7.82 (s, 2H), 7.61 (s, 1H), 7.38 (s, 1H), 7.35 (s, 1H), 7.02 (d, J=8.0 Hz, 1H), 6.43-6.39 (m, 2H), 5.11-5.04 (m, 1H), 4.48-4.41 (m, 1H), 4.01 (t, J=8.0 Hz, 2H), 3.84 (q, J=4.6, 4.2 Hz, 2H), 3.81 (s, 3H), 2.73 (s, 6H), 2.13 (s, 3H), 1.40 (dd, J=6.9, 4.1 Hz, 10H). ¹³C NMR (101 MHz, DMSO-d₆) δ 168.79, 148.96, 146.28, 138.34, 136.51, 135.98, 133.68, 133.45, 131.35, 128.30, 125.13, 124.70, 122.55, 122.01, 120.95, 120.83, 120.44, 113.22, 111.11, 55.70, 54.45, 53.61, 48.83, 39.14, 23.16, 18.82. C₃₁H₃₉N₇O, HRMS calculated for m/z [M+H]⁺: 526.3294 (calculated), 526.3302 (found).

### Example 52: N-(1-(3-(1-ethyl-1H-pyrazol-3-yl)-5-(1-(oxetan-3-yl)-1H-pyrazol-4-yl)phenyl)cyclopropyl)-2-methyl-5-(4-methylpiperazin-1-yl)benzamide

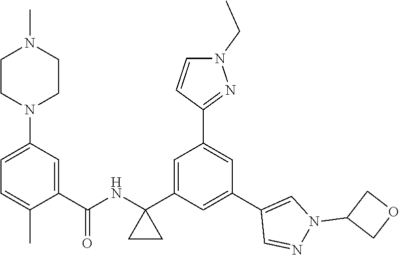 [SMILES: CCn1ccc(-c2cc(-c3cnn(C4COC4)c3)cc(C3(NC(=O)c4cc(N5CCN(C)CC5)ccc4C)CC3)c2)n1]

(Method A) White solid, 85% yield. ¹H NMR (400 MHz, DMSO-d₆) δ 8.91 (s, 1H), 8.40 (s, 1H), 7.98 (s, 1H), 7.72 (s, 2H), 7.61 (s, 1H), 7.19 (s, 1H), 7.07 (d, J=8.1 Hz, 1H), 6.92 (d, J=8.1 Hz, 2H), 6.66 (d, J=2.4 Hz, 1H), 5.57-5.50 (m, 1H), 4.93-4.84 (m, 3H), 4.10 (q, J=7.3 Hz, 2H), 3.74 (dd, J=18.0, 9.5 Hz, 3H), 3.47 (d, J=12.0 Hz, 2H), 3.16-3.03 (m, 2H), 2.89 (t, J=12.3 Hz, 2H), 2.80 (s, 3H), 2.22 (s, 3H), 1.34 (t, J=7.2 Hz, 3H), 1.32-1.28 (m, 2H), 1.25 (q, J=4.5, 4.0 Hz, 2H). ¹³C NMR (101 MHz, DMSO-d₆) δ 170.16, 150.37, 147.70, 144.93, 138.11, 137.47, 134.27, 132.91, 131.70, 131.12, 127.24, 122.94, 120.49, 120.36, 119.87, 117.59, 115.36, 102.90, 77.13, 55.15, 52.75, 46.82, 46.37, 42.57, 34.83, 18.93, 18.57, 15.99. C₃₃H₃₉N₇O₂, HRMS calculated for m/z [M+H]⁺: 566.3243(calculated), 566.3249 (found).

### Example 53: N-((R)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide

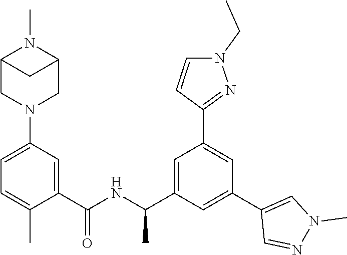 [SMILES: CCn1ccc(-c2cc(-c3cnn(C)c3)cc([C@@H](C)NC(=O)c3cc(N4CC5CC(C4)N5C)ccc3C)c2)n1]

(Method B) White solid, 64% yield. ¹H NMR (400 MHz, DMSO-d₆) δ 8.65 (d, J=8.2 Hz, 1H), 8.12 (s, 1H), 7.82 (s, 1H), 7.74 (s, 1H), 7.72 (d, J=2.3 Hz, 1H), 7.63 (s, 1H), 7.46 (s, 1H), 7.06 (dd, J=8.3, 5.3 Hz, 1H), 6.71-6.63 (m, 3H), 5.14-5.07 (m, 1H), 4.45-4.38 (m, 1H), 4.28-4.22 (m, 1H), 4.10 (q, J=7.3 Hz, 2H), 3.81 (s, 3H), 3.80-3.68 (m, 2H), 3.61 (ddd, J=18.5, 12.2, 6.3 Hz, 2H), 2.97 (d, J=5.0 Hz, 1H), 2.80 (dd, J=11.6, 5.1 Hz, 1H), 2.39 (s, 2H), 2.15 (s, 3H), 1.84 (t, J=9.9 Hz, 1H), 1.43 (d, J=7.0 Hz, 3H), 1.34 (t, J=7.3 Hz, 3H). ¹³C NMR (101 MHz, DMSO-d₆) δ 169.13, 150.36, 146.33, 146.30, 145.67, 144.95, 138.35, 136.46, 134.58, 133.26, 131.62, 131.13, 128.39, 123.67, 123.51, 122.46, 122.36, 120.89, 120.52, 109.97, 109.86, 102.94, 64.83, 62.80, 48.86, 46.81, 43.82, 39.11, 37.12, 29.39, 23.05, 18.72, 18.68, 16.01. C₃₁H₃₇N₇O, HRMS calculated for m/z [M+H]⁺: 524.3138 (calculated), 524.3143 (found).

### Example 54: N-(1-(3-(1-ethyl-1H-pyrazol-3-yl)-5-(1-(2-methoxyethyl)-1H-pyrazol-4-yl)phenyl)cyclopropyl)-2-methyl-5-(4-methylpiperazin-1-yl)benzamide

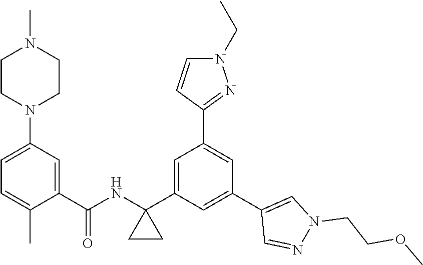 [SMILES: CCn1ccc(-c2cc(-c3cnn(CCOC)c3)cc(C3(NC(=O)c4cc(N5CCN(C)CC5)ccc4C)CC3)c2)n1]

(Method A) White solid, 75% yield. ¹H NMR (400 MHz, DMSO-d₆) δ 8.90 (s, 1H), 8.13 (s, 1H), 7.83 (s, 1H), 7.71 (s, 1H), 7.69 (s, 1H), 7.57 (s, 1H), 7.18 (s, 1H), 7.07 (d, J=8.2 Hz, 1H), 6.92 (d, J=10.1 Hz, 2H), 6.65 (s, 1H), 4.22 (t, J=5.3 Hz, 2H), 4.10 (q, J=7.8, 7.3 Hz, 2H), 3.76 (d, J=13.1 Hz, 2H), 3.66 (t, J=5.4 Hz, 2H), 3.46 (d, J=12.0 Hz, 2H), 3.18 (s, 3H), 3.09 (t, J=16.7 Hz, 2H), 2.89 (t, J=12.7 Hz, 2H), 2.80 (s, 3H), 2.22 (s, 3H), 1.35 (t, J=7.3 Hz, 3H), 1.29 (d, J=5.3 Hz, 2H), 1.24 (d, J=5.2 Hz, 2H). ¹³C NMR (101 MHz, DMSO-d₆) δ 170.13, 150.42, 147.70, 144.86, 138.13, 136.64, 134.29, 133.18, 131.70, 131.09, 128.19, 127.22, 122.34, 120.49, 120.25, 119.77, 117.59, 115.35, 102.90, 70.96, 58.44, 52.77, 51.78, 46.82, 46.37, 42.59, 34.85, 18.91, 18.45, 15.99. C₃₃H₄₁N₇O₂, HRMS calculated for m/z [M+H]⁺: 568.3400 (calculated), 568.3404 (found).

### Example 55: N-(1-(3-(1-(cyanomethyl)-1H-pyrazol-4-yl)-5-(1-ethyl-1H-pyrazol-3-yl)phenyl)cyclopropyl)-2-methyl-5-(4-methylpiperazin-1-yl)benzamide

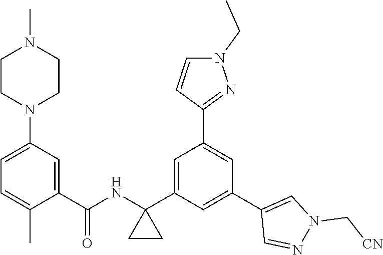 [SMILES: CCn1ccc(-c2cc(-c3cnn(CC#N)c3)cc(C3(NC(=O)c4cc(N5CCN(C)CC5)ccc4C)CC3)c2)n1]

(Method A) White solid, 75% yield. ¹H NMR (400 MHz, DMSO-d₆) δ 8.90 (s, 1H), 8.29 (s, 1H), 8.01 (s, 1H), 7.72 (s, 2H), 7.62 (s, 1H), 7.20 (s, 1H), 7.07 (d, J=8.2 Hz, 1H), 6.92 (d, J=9.4 Hz, 2H), 6.66 (s, 1H), 5.46 (s, 2H), 4.10 (q, J=7.2 Hz, 2H), 3.76 (d, J=13.2 Hz, 2H), 3.46 (d, J=12.0 Hz, 2H), 3.14-3.03 (m, 2H), 2.91 (d, J=12.6 Hz, 2H), 2.80 (s, 3H), 2.22 (s, 3H), 1.34 (t, J=7.2 Hz, 3H), 1.30 (d, J=7.0 Hz, 2H), 1.23 (d, J=8.0 Hz, 2H). ¹³C NMR (101 MHz, DMSO-d₆) δ 170.16, 150.28, 147.71, 145.04, 138.62, 138.10, 134.38, 132.35, 131.70, 131.14, 128.74, 127.24, 123.78, 120.81, 120.69, 120.03, 117.61, 116.38, 115.35, 102.93, 52.78, 46.83, 46.37, 42.60, 34.84, 18.91, 18.50, 15.97. C₃₂H₃₆N₈O, HRMS calculated for m/z [M+H]⁺: 549.3090 (calculated), 549.3101 (found).

### Example 56: N-(1-(3-(1-(difluoromethyl)-1H-pyrazol-3-yl)-5-(1-methyl-1H-pyrazol-4-yl)phenyl)cyclopropyl)-2-methyl-5-(4-methylpiperazin-1-yl)benzamide

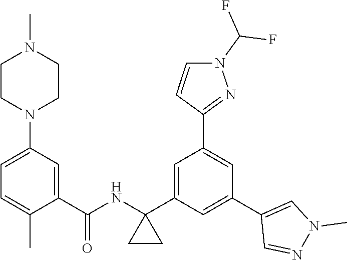 [SMILES: Cc1ccc(N2CCN(C)CC2)cc1C(=O)NC1(c2cc(-c3cnn(C)c3)cc(-c3ccn(C(F)F)n3)c2)CC1]

(Method A) White solid, 89% yield. ¹H NMR (400 MHz, DMSO-d₆) δ 8.92 (s, 1H), 8.24 (s, 1H), 8.15 (s, 1H), 7.84 (s, 1H), 7.77 (d, J=2.2 Hz, 1H), 7.63 (d, J=6.2 Hz, 1H), 7.29 (s, 1H), 7.07 (d, J=8.2 Hz, 1H), 7.01 (s, 1H), 6.92 (d, J=10.9 Hz, 2H), 3.81 (s, 3H), 3.75 (d, J=13.3 Hz, 2H), 3.46 (d, J=12.0 Hz, 2H), 3.10 (t, J=17.3 Hz, 2H), 2.91 (d, J=12.8 Hz, 2H), 2.80 (s, 3H), 2.20 (s, 3H), 1.31 (d, J=5.2 Hz, 2H), 1.26 (d, J=5.4 Hz, 2H). ¹³C NMR (101 MHz, DMSO-d₆) δ 170.20, 154.12, 147.71, 145.28, 138.08, 136.57, 133.54, 132.55, 131.71, 131.29, 128.60, 127.24, 122.26, 121.83, 120.70, 120.55, 117.62, 115.30, 110.72, 106.08, 52.77, 46.36, 42.59, 39.12, 34.84, 18.83, 18.50. C₃₀H₃₃F₂N₇O, HRMS calculated for m/z [M+H]⁺: 546.2793 (calculated), 546.2801 (found).

### Example 57: N-(1-(3-(1-isopropyl-1H-pyrazol-4-yl)-5-(1-methyl-1H-pyrazol-3-yl)phenyl)cyclopropyl)-2-methyl-5-(4-methylpiperazin-1-yl)benzamide

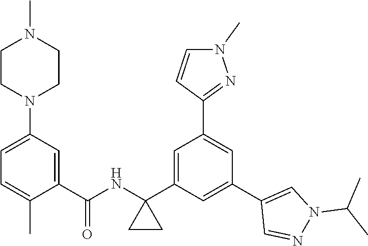 [SMILES: Cc1ccc(N2CCN(C)CC2)cc1C(=O)NC1(c2cc(-c3cnn(C(C)C)c3)cc(-c3ccn(C)n3)c2)CC1]

(Method A) White solid, 85% yield. ¹H NMR (400 MHz, DMSO-d₆) δ 8.89 (s, 1H), 8.19 (s, 1H), 7.80 (s, 1H), 7.70 (s, 1H), 7.67 (s, 1H), 7.55 (s, 1H), 7.19 (s, 1H), 7.07 (d, J=8.2 Hz, 1H), 6.92 (d, J=8.2 Hz, 2H), 6.64 (s, 1H), 4.45 (p, J=6.6 Hz, 1H), 3.81 (s, 3H), 3.76 (d, J=13.3 Hz, 2H), 3.47 (d, J=12.0 Hz, 2H), 3.09 (t, J=14.2 Hz, 2H), 2.90 (t, J=12.7 Hz, 2H), 2.80 (s, 3H), 2.21 (s, 3H), 1.39 (d, J=6.7 Hz, 6H), 1.34 (d, J=6.6 Hz, 1H), 1.29 (d, J=5.3 Hz, 2H), 1.24 (d, J=5.2 Hz, 2H). ¹³C NMR (101 MHz, DMSO-d₆) δ 170.12, 150.64, 147.70, 144.83, 138.14, 135.94, 134.12, 133.40, 132.64, 131.70, 127.21, 125.24, 122.07, 120.50, 120.15, 119.73, 117.60, 115.32, 103.04, 53.61, 52.77, 46.37, 42.59, 39.10, 34.84, 23.15, 18.88, 18.44. C₃₂H₃₉N₇O, HRMS calculated for m/z [M+H]⁺: 538.3294 (calculated), 538.3302 (found).

### Example 58: N-(1-(3-(1-ethyl-1H-pyrazol-3-yl)-5-(1-methyl-1H-pyrazol-3-yl)phenyl)cyclopropyl)-2-methyl-5-(4-methylpiperazin-1-yl)benzamide

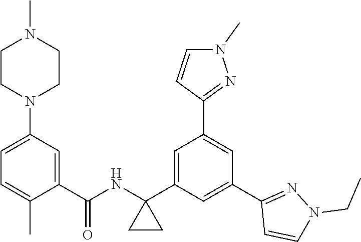 [SMILES: CCn1ccc(-c2cc(-c3ccn(C)n3)cc(C3(NC(=O)c4cc(N5CCN(C)CC5)ccc4C)CC3)c2)n1]

(Method A) White solid, 88% yield. ¹H NMR (400 MHz, DMSO-d₆) δ 8.93 (s, 1H), 7.92 (s, 1H), 7.71 (s, 1H), 7.67 (s, 1H), 7.55 (s, 1H), 7.52 (s, 1H), 7.07 (d, J=8.3 Hz, 1H), 6.95-6.88 (m, 2H), 6.63 (d, J=2.1 Hz, 2H), 4.11 (q, J=6.9 Hz, 2H), 3.82 (s, 3H), 3.75 (d, J=13.3 Hz, 2H), 3.47 (d, J=12.0 Hz, 2H), 3.09 (t, J=15.8 Hz, 2H), 2.89 (t, J=12.4 Hz, 2H), 2.80 (s, 3H), 2.21 (s, 3H), 1.35 (t, J=7.3 Hz, 3H), 1.26 (d, J=7.4 Hz, 4H). ¹³C NMR (101 MHz, DMSO-d₆) δ 170.13, 150.56, 150.37, 147.69, 144.62, 138.11, 134.16, 134.05, 132.72, 131.70, 131.16, 127.28, 121.15, 120.97, 119.77, 117.63, 115.33, 102.99, 102.82, 52.77, 46.83, 46.38, 42.59, 39.11, 34.83, 18.90, 18.37, 16.00. C₃₁H₃₇N₇O, HRMS calculated for m/z [M+H]⁺: 524.3138 (calculated), 524.3141 (found).

### Example 59: N-(1-(3-(1-ethyl-1H-pyrazol-3-yl)-5-(1-methyl-1H-imidazol-4-yl)phenyl)cyclopropyl)-2-methyl-5-(4-methylpiperazin-1-yl)benzamide

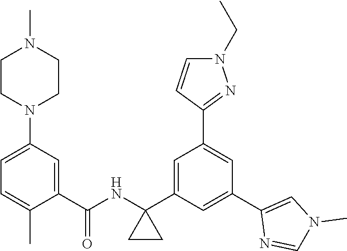 [SMILES: CCn1ccc(-c2cc(-c3cn(C)cn3)cc(C3(NC(=O)c4cc(N5CCN(C)CC5)ccc4C)CC3)c2)n1]

(Method A) White solid, 81% yield. ¹H NMR (400 MHz, DMSO-d₆) δ 9.11 (s, 1H), 8.98 (s, 1H), 8.20 (s, 1H), 7.92 (s, 1H), 7.89 (s, 1H), 7.76 (d, J=2.2 Hz, 1H), 7.29 (s, 1H), 7.08 (d, J=9.0 Hz, 1H), 6.94 (d, J=6.8 Hz, 2H), 6.64 (d, J=2.3 Hz, 1H), 4.11 (q, J=7.3 Hz, 2H), 3.85 (s, 3H), 3.77 (d, J=12.2 Hz, 2H), 3.46 (t, J=14.1 Hz, 2H), 3.15-3.01 (m, 2H), 2.99-2.86 (m, 2H), 2.81 (s, 3H), 2.22 (s, 3H), 1.35 (dd, J=9.1, 5.5 Hz, 5H), 1.31-1.28 (m, 2H). ¹³C NMR (101 MHz, DMSO-d₆) δ 170.31, 149.65, 147.72, 145.82, 137.90, 137.05, 134.66, 133.64, 131.72, 131.46, 127.70, 127.29, 123.45, 119.85, 119.73, 117.66, 115.37, 102.91, 52.73, 46.92, 46.33, 42.57, 36.07, 34.81, 18.93, 18.87, 15.93. C₃₁H₃₇N₇O, HRMS calculated for m/z [M+H]⁺: 524.3138 (calculated), 524.3143 (found).

### Example 60: N-(1-(3,5-bis(1-methyl-1H-pyrazol-4-yl)phenyl)cyclopropyl)-2-methyl-5-(4-methylpiperazin-1-yl)benzamide

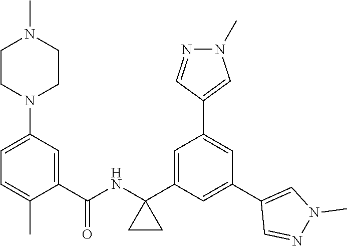 [SMILES: Cc1ccc(N2CCN(C)CC2)cc1C(=O)NC1(c2cc(-c3cnn(C)c3)cc(-c3cnn(C)c3)c2)CC1]

(Method A) White solid, 80% yield. ¹H NMR (400 MHz, DMSO-d₆) δ 8.94 (s, 1H), 8.15 (s, 2H), 7.86 (s, 2H), 7.60 (s, 1H), 7.24 (s, 2H), 7.15 (d, J=8.5 Hz, 1H), 7.00 (d, J=7.2 Hz, 2H), 3.88 (s, 6H), 3.87-3.82 (m, 2H), 3.54 (d, J=12.1 Hz, 2H), 3.16 (t, J=12.8 Hz, 2H), 2.97 (t, J=12.5 Hz, 2H), 2.88 (s, 3H), 2.27 (s, 3H), 1.37 (q, J=5.1, 4.1 Hz, 2H), 1.30 (dt, J=7.9, 4.4 Hz, 2H). ¹³C NMR (101 MHz, DMSO-d₆) δ 170.15, 147.73, 145.14, 138.10, 136.45, 133.28, 131.71, 128.34, 127.08, 122.61, 119.75, 119.62, 117.61, 115.28, 52.78, 46.35, 42.58, 39.12, 34.83, 18.87, 18.45. C₃₀H₃₅N₇O, HRMS calculated for m/z [M+H]⁺: 510.2981 (calculated), 510.2989 (found).

### Example 61: 2-methyl-N-(1-(3-(1-methyl-1H-pyrazol-3-yl)-5-(thiazol-5-yl)phenyl)cyclopropyl)-5-(4-methylpiperazin-1-yl)benzamide

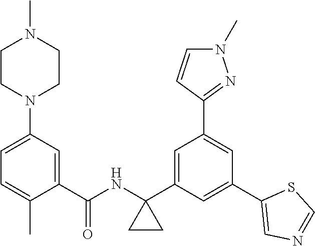 [SMILES: Cc1ccc(N2CCN(C)CC2)cc1C(=O)NC1(c2cc(-c3ccn(C)n3)cc(-c3cncs3)c2)CC1]

(Method A) White solid, 80% yield. ¹H NMR (400 MHz, DMSO-d₆) δ 9.05 (s, 1H), 8.96 (s, 1H), 8.30 (s, 1H), 7.81 (s, 1H), 7.70 (d, J=2.2 Hz, 1H), 7.61 (s, 1H), 7.34 (s, 1H), 7.08 (d, J=8.3 Hz, 1H), 6.96-6.90 (m, 2H), 6.73 (d, J=2.2 Hz, 1H), 4.87 (s, 2H), 3.83 (s, 3H), 3.77 (d, J=13.2 Hz, 2H), 3.48 (d, J=12.0 Hz, 2H), 2.89 (t, J=12.5 Hz, 2H), 2.81 (s, 3H), 2.21 (s, 3H), 1.36-1.30 (m, 2H), 1.29-1.23 (m, 2H). ¹³C NMR (101 MHz, DMSO-d₆) δ 170.22, 154.02, 149.87, 147.73, 145.65, 140.00, 139.26, 137.97, 134.79, 132.91, 131.73, 131.40, 127.18, 122.67, 121.87, 121.01, 117.72, 115.27, 103.36, 52.77, 46.39, 42.59, 39.18, 39.10, 34.75, 18.89, 18.58. C₂₉H₃₂N₆OS, HRMS calculated for m/z [M+H]⁺: 51513.2436 (calculated), 513.2443 (found).

### Example 62: (R)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide

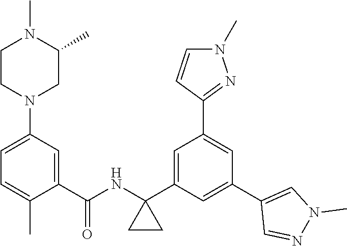 [SMILES: Cc1ccc(N2CCN(C)[C@H](C)C2)cc1C(=O)NC1(c2cc(-c3cnn(C)c3)cc(-c3ccn(C)n3)c2)CC1]

(Method A) White solid, 77% yield. ¹H NMR (400 MHz, DMSO-d₆) δ 8.97 (s, 1H), 8.20 (s, 1H), 7.88 (s, 1H), 7.76 (s, 1H), 7.74 (d, J=2.2 Hz, 1H), 7.61 (s, 1H), 7.27 (s, 1H), 7.15 (d, J=8.3 Hz, 1H), 7.01 (dd, J=8.2, 2.7 Hz, 1H), 6.98 (d, J=2.8 Hz, 1H), 6.72 (d, J=2.2 Hz, 1H), 3.89 (d, J=2.8 Hz, 8H), 3.59 (d, J=12.4 Hz, 1H), 3.35 (d, J=10.7 Hz, 1H), 3.23 (d, J=11.4 Hz, 1H), 2.97 (t, J=11.4 Hz, 1H), 2.88 (s, 2H), 2.81-2.70 (m, 1H), 2.28 (s, 3H), 1.44-1.21 (m, 7H). 13C NMR (101 MHz, DMSO-d₆) δ 170.14, 159.07, 158.71, 150.56, 147.53, 144.89, 138.18, 136.44, 134.17, 133.18, 132.67, 131.68, 128.45, 127.11, 122.54, 120.58, 120.11, 119.71, 117.56, 115.20, 103.02, 59.30, 53.64, 53.11, 46.49, 39.10, 34.82, 18.88, 18.42, 14.49. C₃₁H₃₇N₇O, HRMS calculated for m/z [M+H]⁺: 524.3138 (calculated), 524.3142 (found).

### Example 63: N-(1-(3-(1-(2-fluoroethyl)-1H-pyrazol-4-yl)-5-(1-methyl-1H-pyrazol-3-yl)phenyl)cyclopropyl)-2-methyl-5-(4-methylpiperazin-1-yl)benzamide

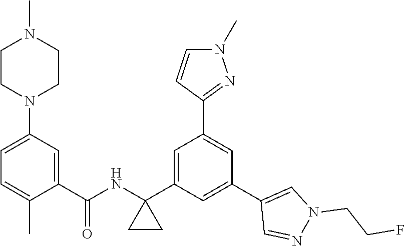 [SMILES: Cc1ccc(N2CCN(C)CC2)cc1C(=O)NC1(c2cc(-c3cnn(CCF)c3)cc(-c3ccn(C)n3)c2)CC1]

(Method A) White solid, 80% yield. ¹H NMR (400 MHz, DMSO-d₆) δ 9.03 (s, 1H), 8.33 (s, 1H), 8.01 (s, 1H), 7.83 (s, 1H), 7.79 (d, J=2.3 Hz, 1H), 7.68 (s, 1H), 7.32 (s, 1H), 7.18 (t, J=6.9 Hz, 1H), 7.04 (d, J=8.2 Hz, 2H), 6.78 (d, J=2.3 Hz, 1H), 4.93 (t, J=4.7 Hz, 1H), 4.81 (t, J=4.7 Hz, 1H), 4.55 (t, J=4.8 Hz, 1H), 4.48 (t, J=4.8 Hz, 1H), 3.94 (s, 3H), 3.88 (d, J=13.2 Hz, 2H), 3.59 (d, J=12.1 Hz, 2H), 3.28-3.15 (m, 2H), 3.01 (t, J=12.4 Hz, 2H), 2.92 (s, 3H), 2.33 (s, 3H), 1.45-1.39 (m, 2H), 1.35 (dd, J=7.1, 4.9 Hz, 2H). ¹³C NMR (101 MHz, DMSO-d₆) δ 170.14, 159.07, 158.72, 150.55, 147.70, 144.93, 138.11, 137.06, 134.18, 133.03, 132.68, 131.70, 128.34, 127.20, 122.66, 120.57, 120.24, 119.75, 117.62, 115.31, 103.05, 83.37, 81.70, 52.75, 52.54, 52.35, 46.37, 42.57, 39.11, 34.82, 18.88, 18.47. C₃₁H₃₆FN₇O, HRMS calculated for m/z [M+H]⁺: 542.3044 (calculated), 542.3050 (found).

### Example 64: 2-methyl-N-(1-(3-(1-methyl-1H-pyrazol-3-yl)-5-(thiazol-2-yl)phenyl)cyclopropyl)-5-(4-methylpiperazin-1-yl)benzamide

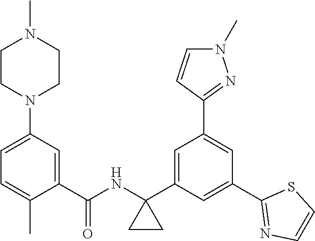 [SMILES: Cc1ccc(N2CCN(C)CC2)cc1C(=O)NC1(c2cc(-c3ccn(C)n3)cc(-c3nccs3)c2)CC1]

(Method A) White solid, 70% yield. ¹H NMR (400 MHz, DMSO-d₆) δ 9.02 (s, 1H), 8.09 (s, 1H), 7.88 (d, J=3.2 Hz, 1H), 7.77 (s, 1H), 7.75 (d, J=3.3 Hz, 1H), 7.71 (d, J=2.3 Hz, 1H), 7.61 (s, 1H), 7.08 (d, J=9.2 Hz, 1H), 6.94 (d, J=5.6 Hz, 2H), 6.72 (d, J=2.3 Hz, 1H), 3.84 (s, 3H), 3.77 (d, J=13.3 Hz, 2H), 3.49 (d, J=11.9 Hz, 2H), 3.11 (t, J=15.7 Hz, 2H), 2.91 (t, J=12.4 Hz, 2H), 2.81 (s, 3H), 2.20 (s, 3H), 1.33 (d, J=2.2 Hz, 2H), 1.30-1.26 (m, 2H). ¹³C NMR (101 MHz, DMSO-d₆) δ 170.25, 167.72, 149.72, 147.73, 145.64, 144.28, 137.95, 134.84, 133.76, 133.01, 131.73, 127.19, 123.13, 122.31, 120.93, 120.54, 117.73, 115.24, 103.28, 52.75, 46.37, 42.58, 39.19, 34.71, 18.86, 18.59. C₂₉H₃₂N₆OS, HRMS calculated for m/z [M+H]⁺: 513.2436 (calculated), 513.2441 (found).

### Example 65: 5-(3,6-diazabicyclo[3.1.1]heptan-3-yl)-N-(1-(3-(1-ethyl-1H-pyrazol-3-yl)-5-(1-methyl-1H-pyrazol-4-yl)phenyl)cyclopropyl)-2-methylbenzamide

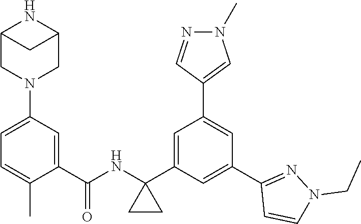 [SMILES: CCn1ccc(-c2cc(-c3cnn(C)c3)cc(C3(NC(=O)c4cc(N5CC6CC(C5)N6)ccc4C)CC3)c2)n1]

(Method A) White solid, 76% yield. ¹H NMR (400 MHz, DMSO-d₆) δ 9.04 (s, 1H), 8.24 (s, 1H), 7.93 (s, 1H), 7.84 (d, J=2.3 Hz, 1H), 7.80 (s, 1H), 7.69 (s, 1H), 7.30 (s, 1H), 7.19 (d, J=8.3 Hz, 1H), 6.81 (d, J=7.1 Hz, 2H), 6.77 (d, J=2.3 Hz, 1H), 4.64 (s, 1H), 4.55 (d, J=5.0 Hz, 2H), 4.22 (q, J=7.2 Hz, 2H), 3.93 (s, 3H), 3.85 (d, J=11.7 Hz, 2H), 3.73 (d, J=11.7 Hz, 2H), 2.95 (dt, J=10.1, 6.4 Hz, 1H), 2.33 (s, 3H), 1.89 (dd, J=10.0, 5.6 Hz, 1H), 1.46 (t, J=7.3 Hz, 3H), 1.41 (d, J=4.1 Hz, 2H), 1.36 (q, J=4.2 Hz, 2H). ¹³C NMR (101 MHz, DMSO-d₆) δ 170.49, 150.41, 145.88, 144.94, 138.20, 136.47, 134.28, 133.18, 131.74, 131.11, 128.43, 123.61, 122.56, 120.38, 120.15, 119.74, 112.07, 109.79, 102.89, 58.31, 48.11, 46.81, 39.10, 34.79, 29.31, 18.81, 18.53, 16.00. C₃₁H₃₅N₇O, HRMS calculated for m/z [M+H]⁺: 522.2981 (calculated), 522.2989 (found).

### Example 66: 2-methyl-N-(1-(3-(1-methyl-1H-pyrazol-3-yl)-5-(1-methyl-1H-pyrazol-4-yl)phenyl)cyclopropyl)-5-(6-methyl-3,6-diazabicyclo[3.1.1]heptan-3-yl)benzamide

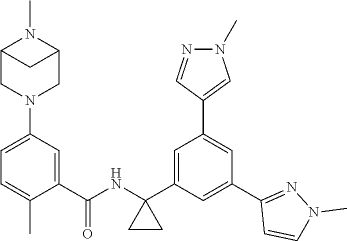 [SMILES: Cc1ccc(N2CC3CC(C2)N3C)cc1C(=O)NC1(c2cc(-c3cnn(C)c3)cc(-c3ccn(C)n3)c2)CC1]

(Method A) White solid, 74% yield. ¹H NMR (400 MHz, DMSO-d₆) δ 9.04 (s, 1H), 8.24 (s, 1H), 7.92 (s, 1H), 7.80 (d, J=3.3 Hz, 2H), 7.68 (s, 1H), 7.31 (s, 1H), 7.20 (t, J=7.2 Hz, 1H), 6.82 (d, J=9.5 Hz, 2H), 6.76 (d, J=2.2 Hz, 1H), 4.56 (d, J=6.1 Hz, 1H), 4.40 (d, J=6.3 Hz, 1H), 3.93 (s, 7H), 3.91-3.84 (m, 2H), 3.76 (dd, J=23.6, 12.3 Hz, 2H), 3.10 (d, J=5.0 Hz, 1H), 2.54-2.50 (m, 2H), 2.33 (s, 3H), 1.99 (d, J=10.2 Hz, 1H), 1.41 (d, J=4.8 Hz, 2H), 1.35 (d, J=4.8 Hz, 2H). ¹³C NMR (101 MHz, DMSO-d₆) δ 170.46, 170.43, 159.02, 158.67, 150.57, 145.70, 145.00, 144.94, 138.27, 138.11, 136.46, 134.17, 133.19, 132.69, 131.75, 128.43, 123.85, 122.55, 120.51, 120.10, 119.70, 118.07, 115.15, 112.02, 109.83, 103.03, 64.83, 62.79, 48.94, 43.84, 39.10, 37.13, 34.78, 29.39, 28.41, 18.75, 18.49. C₃₁H₃₅N₇O, HRMS calculated for m/z [M+H]⁺: 522.2981 (calculated), 522.2990 (found).

### Example 67: N-(1-(3-(1-ethyl-1H-pyrazol-3-yl)-5-(1-methyl-1H-pyrazol-4-yl)phenyl)cyclopropyl)-2-methyl-5-(6-methyl-3,6-diazabicyclo[3.1.1]heptan-3-yl)benzamide

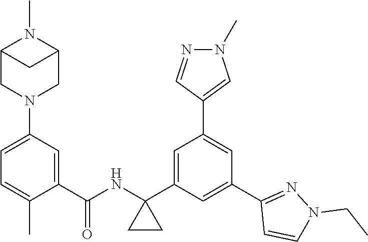 [SMILES: CCn1ccc(-c2cc(-c3cnn(C)c3)cc(C3(NC(=O)c4cc(N5CC6CC(C5)N6C)ccc4C)CC3)c2)n1]

(Method A) White solid, 79% yield. ¹H NMR (400 MHz, DMSO-d₆) δ 8.92 (s, 1H), 8.12 (s, 1H), 7.81 (s, 1H), 7.71 (d, J=2.2 Hz, 1H), 7.68 (s, 1H), 7.57 (s, 1H), 7.18 (s, 1H), 7.10-7.05 (m, 1H), 6.71 (s, 1H), 6.70-6.67 (m, 1H), 6.64 (d, J=2.3 Hz, 1H), 4.44 (d, J=6.1 Hz, 1H), 4.27 (d, J=6.2 Hz, 1H), 4.09 (q, J=7.3 Hz, 2H), 3.81 (s, 3H), 3.78-3.56 (m, 4H), 2.98 (d, J=5.0 Hz, 1H), 2.40 (d, J=3.4 Hz, 2H), 2.21 (s, 3H), 1.86 (d, J=10.2 Hz, 1H), 1.34 (t, J=7.3 Hz, 3H), 1.29 (d, J=4.9 Hz, 2H), 1.26-1.21 (m, 2H). ¹³C NMR (101 MHz, DMSO-d₆) δ 170.47, 170.44, 159.04, 158.68, 150.40, 145.70, 145.00, 144.92, 138.09, 136.48, 134.29, 133.18, 131.74, 131.11, 128.43, 123.88, 122.56, 120.41, 120.18, 119.75, 115.04, 112.03, 109.84, 102.89, 64.83, 62.79, 48.94, 46.81, 43.84, 39.10, 37.14, 34.80, 29.39, 28.40, 18.79, 18.50, 15.99. C₃₂H₃₇N₇O, HRMS calculated for m/z [M+H]⁺: 536.3138 (calculated), 536.3142 (found).

### Example 68: N-(1-(3-(1-ethyl-1H-pyrazol-4-yl)-5-(1-methyl-1H-pyrazol-3-yl)phenyl)cyclopropyl)-2-methyl-5-(6-methyl-3,6-diazabicyclo[3.1.1]heptan-3-yl)benzamide

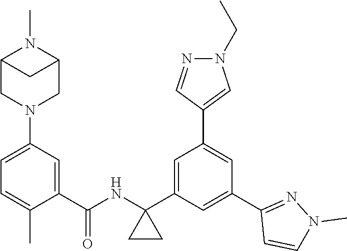 [SMILES: CCn1cc(-c2cc(-c3ccn(C)n3)cc(C3(NC(=O)c4cc(N5CC6CC(C5)N6C)ccc4C)CC3)c2)cn1]

(Method A) White solid, 78% yield. ¹H NMR (400 MHz, DMSO-d₆) δ 8.91 (s, 1H), 8.17 (s, 1H), 7.81 (s, 1H), 7.69 (s, 1H), 7.67 (d, J=2.2 Hz, 1H), 7.56 (s, 1H), 7.20 (s, 1H), 7.07 (t, J=7.2 Hz, 1H), 6.70 (d, J=9.4 Hz, 2H), 6.64 (d, J=2.2 Hz, 1H), 4.44 (d, J=6.1 Hz, 1H), 4.28 (d, J=6.3 Hz, 1H), 4.09 (q, J=7.3 Hz, 2H), 3.81 (s, 3H), 3.79-3.58 (m, 4H), 2.98 (d, J=5.0 Hz, 1H), 2.81 (dd, J=11.5, 5.3 Hz, 1H), 2.40 (d, J=3.8 Hz, 2H), 2.21 (s, 3H), 1.87 (d, J=10.1 Hz, 1H), 1.35 (t, J=7.3 Hz, 3H), 1.32-1.27 (m, 2H), 1.23 (q, J=5.8, 4.9 Hz, 2H). ¹³C NMR (101 MHz, DMSO-d₆) δ 170.46, 170.44, 159.03, 158.68, 150.60, 145.70, 145.00, 144.92, 138.27, 138.11, 136.29, 134.15, 133.29, 132.68, 131.75, 126.94, 123.85, 122.32, 120.12, 119.71, 112.18, 112.02, 109.84, 103.03, 64.83, 62.79, 48.94, 46.85, 43.85, 39.10, 37.14, 34.80, 29.39, 18.77, 18.75, 18.47, 15.87. C₃₂H₃₇N₇O, HRMS calculated for m/z [M+H]⁺: 536.3138 (calculated), 536.3144 (found).

### Example 69: N-(1-(3-(1-ethyl-1H-pyrazol-3-yl)-5-(1-methyl-1H-pyrazol-3-yl)phenyl)cyclopropyl)-2-methyl-5-(6-methyl-3,6-diazabicyclo[3.1.1]heptan-3-yl)benzamide

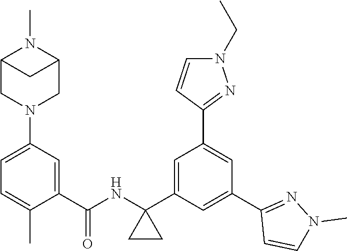 [SMILES: CCn1ccc(-c2cc(-c3ccn(C)n3)cc(C3(NC(=O)c4cc(N5CC6CC(C5)N6C)ccc4C)CC3)c2)n1]

(Method A) White solid, 77% yield. ¹H NMR (400 MHz, DMSO-d₆) δ 8.95 (s, 1H), 7.92 (s, 1H), 7.72 (d, J=2.3 Hz, 1H), 7.67 (d, J=2.2 Hz, 1H), 7.56 (s, 1H), 7.53 (s, 1H), 7.11-7.04 (m, 1H), 6.71 (s, 1H), 6.70-6.67 (m, 1H), 6.64 (s, 2H), 4.44 (d, J=6.1 Hz, 1H), 4.27 (d, J=6.3 Hz, 1H), 4.11 (q, J=7.3 Hz, 2H), 3.82 (s, 3H), 3.81-3.58 (m, 4H), 2.98 (d, J=5.1 Hz, 1H), 2.81 (dt, J=10.4, 6.4 Hz, 1H), 2.40 (d, J=3.7 Hz, 2H), 2.21 (s, 3H), 1.86 (d, J=10.2 Hz, 1H), 1.34 (t, J=7.2 Hz, 3H), 1.27 (d, J=3.4 Hz, 2H), 1.25 (d, J=2.5 Hz, 2H). ¹³C NMR (101 MHz, DMSO-d₆) δ 170.47, 170.43, 150.55, 150.37, 145.69, 144.98, 144.68, 144.66, 138.26, 138.09, 134.16, 134.04, 132.75, 131.74, 131.18, 123.92, 123.72, 121.14, 120.96, 119.75, 109.91, 109.84, 102.99, 102.82, 64.82, 62.77, 48.93, 46.82, 43.85, 39.11, 37.14, 34.81, 29.38, 28.40, 18.79, 18.42, 16.03. C₃₂H₃₇N₇O, HRMS calculated for m/z [M+H]⁺: 536.3138 (calculated), 536.3145 (found).

### Example 70: (S)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide

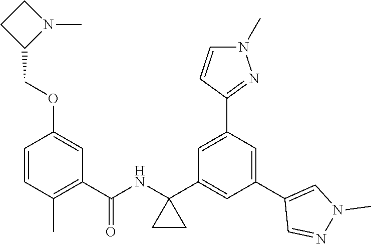

(Method A) White solid, 81% yield. ¹H NMR (400 MHz, DMSO-d₆) δ 9.03 (s, 1H), 8.19 (s, 1H), 7.88 (s, 1H), 7.76 (s, 1H), 7.74 (d, J=2.2 Hz, 1H), 7.62 (s, 1H), 7.27 (d, J=7.6 Hz, 1H), 7.21 (d, J=9.3 Hz, 1H), 7.02-6.93 (m, 2H), 6.72 (d, J=2.2 Hz, 1H), 4.69 (q, J=6.6 Hz, 1H), 4.34 (d, J=5.3 Hz, 1H), 4.08 (m, 1H), 3.89 (d, J=3.7 Hz, 7H), 3.73 (d, J=14.1 Hz, 1H), 3.43-3.10 (m, 1H), 2.96-2.87 (m, 3H), 2.46-2.37 (m, 1H), 2.31 (d, J=2.0 Hz, 3H), 1.40-1.33 (m, 2H), 1.30 (d, J=4.4 Hz, 2H). ¹³C NMR (101 MHz, DMSO-d₆) δ 169.66, 155.78, 150.54, 144.77, 138.67, 136.46, 134.20, 133.20, 132.67, 132.02, 128.45, 128.35, 122.53, 120.63, 120.18, 119.77, 115.86, 115.24, 114.14, 103.04, 68.26, 67.08, 53.07, 41.45, 39.10, 34.85, 18.89, 18.87, 18.30. C₃₀H₃₄N₆O₂, HRMS calculated for m/z [M+H]⁺: 511.2821 (calculated), 511.2829 (found).

### Example 71: (S)-N-(1-(3,5-bis(1-methyl-1H-pyrazol-4-yl)phenyl)cyclopropyl)-2-methyl-5-((1-methylazetidin-2-yl)methoxy)benzamide

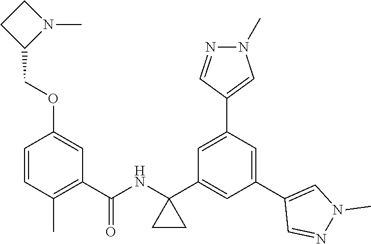

(Method A) White solid, 88% yield. ¹H NMR (400 MHz, DMSO-d₆) δ 8.91 (s, 1H), 8.07 (d, J=2.0 Hz, 2H), 7.79 (s, 2H), 7.52 (s, 1H), 7.17 (d, J=5.1 Hz, 2H), 7.13 (d, J=8.0 Hz, 1H), 6.93 (s, 1H), 6.92-6.85 (m, 1H), 4.62 (q, J=7.6, 6.5 Hz, 1H), 4.27 (d, J=5.3 Hz, 1H), 4.07-3.96 (m, 1H), 3.81 (s, 7H), 3.64 (d, J=9.0 Hz, 1H), 3.34-3.04 (m, 1H), 2.82 (t, J=5.3 Hz, 3H), 2.35 (dd, J=11.4, 7.0 Hz, 1H), 2.22 (d, J=2.1 Hz, 3H), 1.30 (q, J=4.9, 4.1 Hz, 2H), 1.21 (q, J=5.0, 4.6 Hz, 2H). ¹³C NMR (101 MHz, DMSO-d₆) δ 169.67, 155.81, 145.04, 138.66, 136.45, 133.31, 132.03, 128.36, 128.22, 122.59, 119.81, 119.66, 117.85, 115.90, 114.94, 114.08, 68.27, 67.09, 53.07, 41.47, 39.12, 34.85, 18.88, 18.86, 18.33. C₃₀H₃₄N₆O₂, HRMS calculated for m/z [M+H]⁺: 511.2821 (calculated), 511.2830 (found).

### Example 72: N-(1-(3-(1-ethyl-1H-pyrazol-3-yl)-5-(1-(oxetan-3-yl)-1H-pyrazol-3-yl)phenyl)cyclopropyl)-2-methyl-5-(4-methylpiperazin-1-yl)benzamide

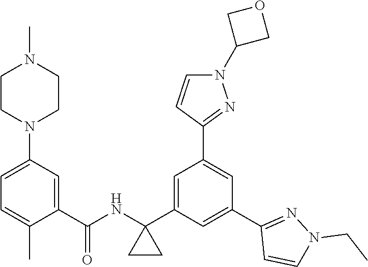 [SMILES: CCn1ccc(-c2cc(-c3ccn(C4COC4)n3)cc(C3(NC(=O)c4cc(N5CCN(C)CC5)ccc4C)CC3)c2)n1]

(Method A) White solid, 83% yield. ¹H NMR (400 MHz, DMSO-d₆) δ 8.96 (s, 1H), 7.97 (s, 1H), 7.89 (d, J=2.3 Hz, 1H), 7.73 (d, J=2.3 Hz, 1H), 7.60 (s, 1H), 7.56 (s, 1H), 7.07 (d, J=8.2 Hz, 1H), 6.92 (dd, J=10.7, 2.1 Hz, 2H), 6.74 (d, J=2.3 Hz, 1H), 6.66 (d, J=2.2 Hz, 1H), 5.56 (p, J=7.0 Hz, 1H), 4.93-4.86 (m, 4H), 4.12 (q, J=7.3 Hz, 2H), 3.75 (d, J=12.4 Hz, 2H), 3.46 (d, J=12.0 Hz, 2H), 3.09 (t, J=13.6 Hz, 2H), 2.89 (t, J=12.4 Hz, 2H), 2.23 (s, 3H), 1.35 (t, J=7.3 Hz, 3H), 1.29 (s, 2H), 1.26 (d, J=4.5 Hz, 2H). ¹³C NMR (101 MHz, DMSO-d₆) δ 170.17, 151.48, 150.31, 147.72, 144.73, 138.10, 134.25, 133.83, 131.74, 131.69, 131.18, 127.28, 121.34, 121.25, 120.00, 117.62, 115.38, 103.57, 102.93, 77.13, 55.03, 52.76, 46.83, 46.38, 42.58, 34.86, 18.93, 18.47, 16.03. C₃₃H₃₉N₇O₂, HRMS calculated for m/z [M+H]⁺: 566.3243 (calculated), 566.3249 (found).

### Example 73: N-(1-(3-(1-ethyl-1H-pyrazol-3-yl)-5-(1-methyl-1H-pyrazol-4-yl)phenyl)cyclopropyl)-2-methyl-5-(3-methyl-3,6-diazabicyclo[3.1.1]heptan-6-yl)benzamide

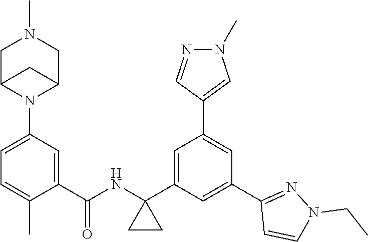 [SMILES: CCn1ccc(-c2cc(-c3cnn(C)c3)cc(C3(NC(=O)c4cc(N5C6CC5CN(C)C6)ccc4C)CC3)c2)n1]

(Method A) White solid, 75% yield. ¹H NMR (400 MHz, DMSO-d₆) δ 8.89 (s, 1H), 8.12 (s, 1H), 7.80 (s, 1H), 7.71 (d, J=2.2 Hz, 1H), 7.68 (s, 1H), 7.56 (s, 1H), 7.17 (s, 1H), 7.08 (d, J=8.1 Hz, 1H), 6.64 (d, J=2.3 Hz, 1H), 6.55-6.49 (m, 2H), 4.31 (d, J=5.9 Hz, 2H), 4.10 (q, J=7.3 Hz, 2H), 3.81 (s, 3H), 3.55 (d, J=12.7 Hz, 2H), 3.33 (dd, J=13.4, 4.3 Hz, 2H), 2.66 (dd, J=10.1, 5.6 Hz, 1H), 2.51 (s, 3H), 2.21 (s, 3H), 1.98 (d, J=9.7 Hz, 1H), 1.34 (t, J=7.3 Hz, 3H), 1.30 (t, J=3.1 Hz, 2H), 1.27-1.21 (m, 2H). ¹³C NMR (101 MHz, DMSO-d₆) δ 170.07, 150.40, 144.85, 142.30, 138.64, 136.48, 134.30, 133.19, 132.12, 131.12, 128.46, 125.43, 122.55, 120.40, 120.16, 119.80, 117.87, 116.07, 114.96, 113.58, 102.90, 57.99, 49.83, 46.81, 43.29, 39.09, 34.80, 25.27, 19.01, 18.57, 15.98. C₃₂H₃₇N₇O, HRMS calculated for m/z [M+H]⁺: 536.3138 (calculated), 536.3146 (found).

### Example 74: (S)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide

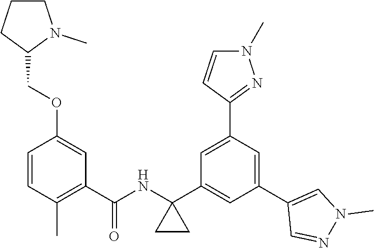

(Method A) White solid, 84% yield. ¹H NMR (400 MHz, DMSO-d₆) δ 9.03 (s, 1H), 8.19 (s, 1H), 7.88 (s, 1H), 7.76 (s, 1H), 7.74 (d, J=2.2 Hz, 1H), 7.62 (s, 1H), 7.27 (d, J=4.8 Hz, 1H), 7.21 (d, J=7.4 Hz, 1H), 7.04-6.97 (m, 2H), 6.72 (d, J=2.2 Hz, 1H), 4.39-4.20 (m, 1H), 3.89 (d, J=3.5 Hz, 6H), 3.81 (d, J=8.8 Hz, 1H), 3.62 (dd, J=16.7, 11.3 Hz, 1H), 3.31 (t, J=11.6 Hz, 1H), 3.20-3.00 (m, 1H), 2.98 (d, J=4.3 Hz, 1H), 2.80 (d, J=4.6 Hz, 1H), 2.31 (s, 3H), 2.29-2.14 (m, 1H), 2.07 (dq, J=7.8, 5.1, 2.9 Hz, 1H), 2.01-1.79 (m, 2H), 1.77-1.58 (m, 1H), 1.36 (d, J=4.7 Hz, 2H), 1.30 (q, J=5.0, 4.4 Hz, 2H). ¹³C NMR (101 MHz, DMSO-d₆) δ 169.66, 155.77, 150.54, 144.78, 138.74, 138.66, 136.46, 134.20, 133.20, 132.67, 128.45, 128.36, 122.53, 120.63, 120.17, 119.77, 115.89, 115.79, 114.06, 103.03, 69.22, 67.25, 66.71, 57.08, 55.96, 53.82, 43.85, 41.16, 39.09, 34.85, 34.83, 26.76, 24.39, 22.51, 18.89, 18.31, 18.03. C₃₁H₃₆N₆O₂, HRMS calculated for m/z [M+H]⁺: 525.2978 (calculated), 525.2984 (found).

### Example 75: N-(1-(3-(1-ethyl-1H-pyrazol-4-yl)-5-(1-methyl-1H-pyrazol-3-yl)phenyl)cyclopropyl)-2-methyl-5-(4-methylpiperazin-1-yl)benzamide

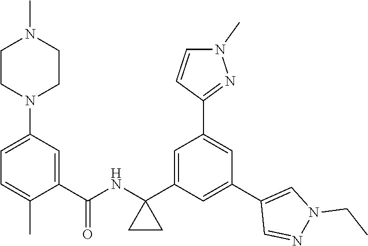 [SMILES: CCn1cc(-c2cc(-c3ccn(C)n3)cc(C3(NC(=O)c4cc(N5CCN(C)CC5)ccc4C)CC3)c2)cn1]

(Method A) White solid, 82% yield. ¹H NMR (400 MHz, DMSO-d₆) δ 8.90 (s, 1H), 8.17 (s, 1H), 7.81 (s, 1H), 7.69 (s, 1H), 7.67 (d, J=2.2 Hz, 1H), 7.55 (s, 1H), 7.18 (s, 1H), 7.07 (d, J=8.2 Hz, 1H), 6.92 (d, J=8.2 Hz, 2H), 6.64 (d, J=2.2 Hz, 1H), 4.09 (q, J=7.2 Hz, 2H), 3.81 (s, 3H), 3.76 (d, J=13.2 Hz, 2H), 3.47 (d, J=12.0 Hz, 2H), 3.09 (t, J=16.3 Hz, 2H), 2.89 (t, J=12.6 Hz, 2H), 2.80 (s, 3H), 2.21 (s, 3H), 1.35 (t, J=7.2 Hz, 3H), 1.28 (d, J=5.1 Hz, 2H), 1.23 (t, J=6.1 Hz, 2H). ¹³C NMR (101 MHz, DMSO-d₆) δ 170.13, 150.60, 147.70, 144.86, 138.12, 136.29, 134.14, 133.29, 132.67, 131.70, 127.21, 126.95, 122.32, 120.49, 120.13, 119.71, 117.62, 115.31, 103.03, 52.76, 46.86, 46.37, 42.58, 39.11, 34.82, 18.88, 18.44, 15.86. C₃₁H₃₇N₇O, HRMS calculated for m/z [M+H]⁺: 524.3138 (calculated), 524.3147 (found).

### Example 76: N-(1-(3-(1-(methoxymethyl)-1H-pyrazol-4-yl)-5-(1-methyl-1H-pyrazol-3-yl)phenyl)cyclopropyl)-2-methyl-5-(4-methylpiperazin-1-yl)benzamide

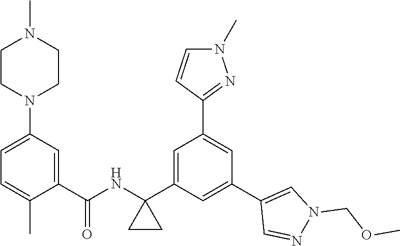 [SMILES: COCn1cc(-c2cc(-c3ccn(C)n3)cc(C3(NC(=O)c4cc(N5CCN(C)CC5)ccc4C)CC3)c2)cn1]

(Method A) White solid, 83% yield. ¹H NMR (400 MHz, DMSO-d₆) δ 8.91 (s, 1H), 8.35 (s, 1H), 7.95 (s, 1H), 7.73 (s, 1H), 7.67 (d, J=2.2 Hz, 1H), 7.59 (s, 1H), 7.22 (s, 1H), 7.07 (d, J=8.1 Hz, 1H), 6.92 (d, J=7.7 Hz, 2H), 6.66 (d, J=2.2 Hz, 1H), 5.34 (s, 2H), 3.82 (s, 3H), 3.76 (d, J=13.2 Hz, 2H), 3.47 (d, J=12.0 Hz, 2H), 3.21 (s, 3H), 3.08 (d, J=12.6 Hz, 2H), 2.96-2.85 (m, 2H), 2.80 (s, 3H), 2.21 (s, 3H), 1.31 (d, J=7.9 Hz, 2H), 1.23 (t, J=6.1 Hz, 2H). ¹³C NMR (101 MHz, DMSO-d₆) δ 170.15, 150.52, 147.70, 144.98, 138.11, 137.86, 134.21, 132.79, 132.69, 131.71, 128.57, 127.21, 123.38, 120.67, 120.50, 119.93, 117.63, 115.31, 103.08, 81.79, 56.60, 52.76, 46.37, 42.58, 39.12, 34.83, 18.88, 18.46. C₃₁H₃₇N₇O₂, HRMS calculated for m/z [M+H]⁺: 540.3087 (calculated), 540.3095 (found).

### Example 77: 2-methyl-N-((R)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide

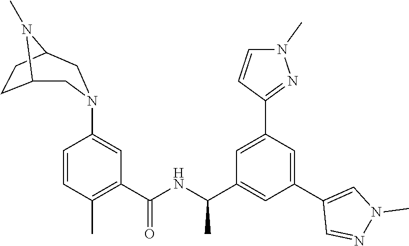 [SMILES: Cc1ccc(N2CC3CCC(C2)N3C)cc1C(=O)N[C@H](C)c1cc(-c2cnn(C)c2)cc(-c2ccn(C)n2)c1]

(Method B) White solid, 79% yield. ¹H NMR (400 MHz, DMSO-d₆) δ 8.64 (dt, J=6.5, 3.0 Hz, 1H), 8.11 (d, J=3.0 Hz, 1H), 7.81 (d, J=3.0 Hz, 1H), 7.74 (s, 1H), 7.67 (s, 1H), 7.61 (s, 1H), 7.44 (s, 1H), 7.02 (dt, J=6.4, 3.0 Hz, 1H), 6.79 (dt, J=10.4, 3.5 Hz, 2H), 6.65 (d, J=3.5 Hz, 1H), 5.09 (dd, J=9.9, 4.9 Hz, 1H), 3.96 (s, 2H), 3.81 (s, 6H), 3.61 (d, J=12.7 Hz, 2H), 3.02 (d, J=12.4 Hz, 2H), 2.70 (s, 3H), 2.12 (q, J=5.3, 3.1 Hz, 5H), 1.93-1.84 (m, 2H), 1.41 (dd, J=7.1, 3.2 Hz, 3H). ¹³C NMR (101 MHz, DMSO-d₆) δ 168.92, 150.53, 147.63, 146.30, 138.38, 136.45, 134.46, 133.27, 132.70, 131.43, 128.38, 125.99, 122.44, 122.38, 120.87, 120.45, 115.90, 113.96, 103.06, 62.57, 51.98, 48.86, 39.10, 38.73, 23.84, 23.08, 18.69. C₃₁H₃₇N₇O, HRMS calculated for m/z [M+H]⁺: 524.3138 (calculated), 524.3145 (found).

### Example 78: 2-methyl-N-(1-(3-(1-methyl-1H-pyrazol-3-yl)-5-(1-methyl-1H-pyrazol-4-yl)phenyl)cyclopropyl)-5-((1R,5S)-8-methyl-3,8-diazabicyclo[3.2.1]octan-3-yl)benzamide

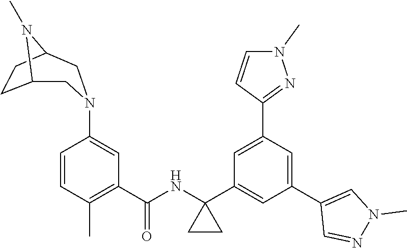 [SMILES: Cc1ccc(N2CC3CCC(C2)N3C)cc1C(=O)NC1(c2cc(-c3cnn(C)c3)cc(-c3ccn(C)n3)c2)CC1]

(Method A) White solid, 76% yield. ¹H NMR (400 MHz, DMSO-d₆) δ 8.89 (s, 1H), 8.11 (s, 1H), 7.80 (s, 1H), 7.68 (s, 1H), 7.67 (s, 1H), 7.53 (s, 1H), 7.19 (s, 1H), 7.04 (d, J=8.3 Hz, 1H), 6.82 (d, J=7.4 Hz, 2H), 6.64 (d, J=2.2 Hz, 1H), 3.99 (s, 2H), 3.81 (d, J=2.9 Hz, 6H), 3.64 (d, J=10.3 Hz, 2H), 3.04 (d, J=12.4 Hz, 2H), 2.71 (d, J=4.7 Hz, 3H), 2.19 (s, 3H), 2.14 (t, J=6.5 Hz, 2H), 1.91 (q, J=5.6 Hz, 2H), 1.28 (d, J=5.3 Hz, 2H), 1.23 (d, J=5.3 Hz, 2H). 13C NMR (101 MHz, DMSO-d₆) δ 170.23, 150.57, 147.63, 144.90, 138.11, 136.45, 134.17, 133.18, 132.67, 131.58, 128.43, 126.21, 122.55, 120.55, 120.11, 119.70, 115.99, 113.95, 103.01, 62.58, 51.98, 39.10, 38.75, 34.80, 23.85, 18.79, 18.43. C₃₂H₃₇N₇O, HRMS calculated for m/z [M+H]⁺: 536.3138 (calculated), 536.3145 (found).

### Example 79: 2-methyl-N-(1-(3-(1-methyl-1H-pyrazol-3-yl)-5-(5-methylthiazol-2-yl)phenyl)cyclopropyl)-5-(4-methylpiperazin-1-yl)benzamide

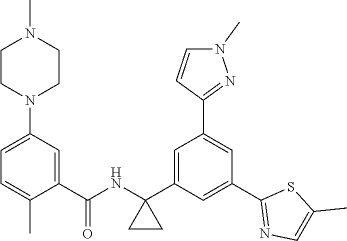 [SMILES: Cc1cnc(-c2cc(-c3ccn(C)n3)cc(C3(NC(=O)c4cc(N5CCN(C)CC5)ccc4C)CC3)c2)s1]

(Method A) White solid, 79% yield. ¹H NMR (400 MHz, DMSO-d₆) δ 8.96 (s, 1H), 8.02 (s, 1H), 7.74 (s, 1H), 7.69 (d, J=2.3 Hz, 1H), 7.65 (d, J=2.2 Hz, 1H), 7.58 (s, 1H), 7.27 (s, 1H), 7.08 (d, J=8.3 Hz, 1H), 6.94 (d, J=2.7 Hz, 1H), 6.92 (t, J=3.7 Hz, 2H), 6.71 (d, J=2.3 Hz, 1H), 3.83 (s, 3H), 3.80 (s, 1H), 3.77 (d, J=13.4 Hz, 2H), 3.47 (d, J=12.0 Hz, 2H), 2.95-2.87 (m, 2H), 2.80 (s, 3H), 2.62 (s, 3H), 2.20 (d, J=5.4 Hz, 3H), 1.34-1.28 (m, 2H), 1.28-1.23 (m, 2H). ¹³C NMR (101 MHz, DMSO-d₆) δ 170.21, 165.23, 149.90, 147.71, 145.56, 138.72, 138.67, 137.96, 134.71, 132.89, 131.73, 131.70, 127.18, 122.22, 121.55, 120.61, 117.68, 115.26, 103.30, 52.75, 46.36, 42.57, 39.16, 39.09, 34.72, 19.45, 18.89, 18.57. C₃₀H₃₄N₆OS, HRMS calculated for m/z [M+H]⁺: 527.2593 (calculated), 527.2600 (found).

### Example 80: 2-methyl-N-(1-(3-(1-methyl-1H-pyrazol-3-yl)-5-(4-methylthiazol-5-yl)phenyl)cyclopropyl)-5-(4-methylpiperazin-1-yl)benzamide

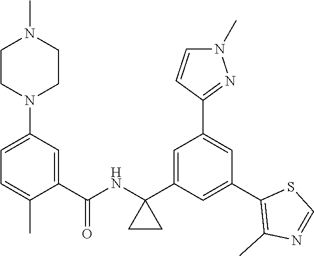 [SMILES: Cc1ccc(N2CCN(C)CC2)cc1C(=O)NC1(c2cc(-c3ccn(C)n3)cc(-c3scnc3C)c2)CC1]

(Method A) White solid, 70% yield. ¹H NMR (400 MHz, DMSO-d₆) δ 8.96 (s, 2H), 7.68 (d, J=2.3 Hz, 1H), 7.61 (s, 1H), 7.59 (s, 1H), 7.21 (s, 1H), 7.07 (d, J=8.2 Hz, 1H), 6.93-6.89 (m, 2H), 6.68 (d, J=2.3 Hz, 1H), 3.82 (s, 3H), 3.80 (s, 1H), 3.78-3.72 (m, 2H), 3.48 (d, J=12.0 Hz, 2H), 3.09 (dd, J=21.6, 11.7 Hz, 2H), 2.89 (t, J=12.5 Hz, 2H), 2.80 (s, 3H), 2.19 (s, 3H), 1.29 (d, J=2.6 Hz, 2H), 1.27 (d, J=2.6 Hz, 2H), 1.22 (s, 1H). ¹³C NMR (101 MHz, DMSO-d₆) δ 170.20, 170.09, 152.08, 149.92, 148.49, 147.73, 147.71, 145.22, 137.87, 134.49, 132.92, 132.06, 131.86, 131.75, 127.24, 125.02, 123.36, 121.32, 117.77, 115.27, 103.28, 52.76, 46.39, 42.57, 39.17, 39.09, 34.75, 18.89, 18.66, 16.50. C₃₀H₃₄N₆OS, HRMS calculated for m/z [M+H]⁺: 527.2593 (calculated), 527.2600 (found).

### Example 81: 5-(3,6-diazabicyclo[3.1.1]heptan-6-yl)-N-(1-(3-(1-ethyl-1H-pyrazol-3-yl)-5-(1-methyl-1H-pyrazol-4-yl)phenyl)cyclopropyl)-2-methylbenzamide

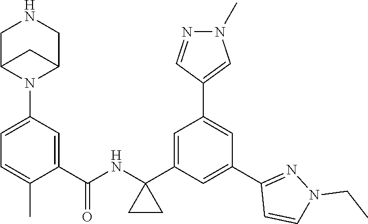 [SMILES: CCn1ccc(-c2cc(-c3cnn(C)c3)cc(C3(NC(=O)c4cc(N5C6CNCC5C6)ccc4C)CC3)c2)n1]

(Method A) White solid, 75% yield. ¹H NMR (400 MHz, DMSO-d₆) δ 8.90 (s, 1H), 8.12 (s, 1H), 7.80 (s, 1H), 7.71 (d, J=2.3 Hz, 1H), 7.67 (s, 1H), 7.55 (s, 1H), 7.17 (s, 1H), 7.07 (d, J=8.2 Hz, 1H), 6.64 (d, J=2.3 Hz, 1H), 6.53 (d, J=7.1 Hz, 2H), 4.28 (d, J=6.1 Hz, 2H), 4.10 (q, J=7.3 Hz, 2H), 3.81 (s, 3H), 3.49 (dt, J=13.1, 6.4 Hz, 2H), 3.13 (dd, J=13.0, 6.3 Hz, 2H), 2.70 (dt, J=11.8, 6.3 Hz, 1H), 2.20 (s, 3H), 1.89 (d, J=9.4 Hz, 1H), 1.34 (t, J=7.3 Hz, 3H), 1.29 (q, J=5.2, 4.0 Hz, 2H), 1.25-1.20 (m, 2H). ¹³C NMR (101 MHz, DMSO-d₆) δ 170.01, 150.41, 144.88, 143.00, 138.59, 136.47, 134.29, 133.16, 132.04, 131.11, 128.44, 125.29, 122.55, 120.43, 120.13, 119.76, 116.14, 113.59, 102.90, 57.63, 46.81, 39.09, 38.89, 34.81, 25.69, 18.99, 18.51, 15.98. C₃₁H₃₅N₇O, HRMS calculated for m/z [M+H]⁺: 522.2981 (calculated), 522.2990 (found).

### Example 82: (R)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide

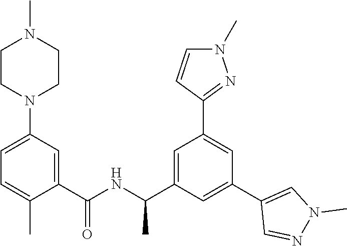 [SMILES: Cc1ccc(N2CCN(C)CC2)cc1C(=O)N[C@H](C)c1cc(-c2cnn(C)c2)cc(-c2ccn(C)n2)c1]

(Method B) White solid, 89% yield. ¹H NMR (400 MHz, DMSO-d₆) δ 8.66 (d, J=8.2 Hz, 1H), 8.12 (s, 1H), 7.82 (s, 1H), 7.74 (s, 1H), 7.68 (d, J=2.3 Hz, 1H), 7.62 (s, 1H), 7.45 (s, 1H), 7.05 (d, J=8.4 Hz, 1H), 6.91 (dd, J=8.4, 2.7 Hz, 1H), 6.87 (d, J=2.7 Hz, 1H), 6.66 (d, J=2.3 Hz, 1H), 5.09 (p, J=7.1 Hz, 1H), 3.82 (d, J=2.6 Hz, 6H), 3.74 (d, J=13.2 Hz, 2H), 3.45 (d, J=11.8 Hz, 2H), 3.08 (d, J=10.6 Hz, 2H), 2.87 (t, J=12.5 Hz, 2H), 2.79 (s, 3H), 2.14 (s, 3H), 1.42 (d, J=7.0 Hz, 3H). ¹³C NMR (101 MHz, DMSO-d₆) δ 168.80, 150.53, 147.69, 146.29, 138.38, 136.45, 134.47, 133.28, 132.71, 131.56, 128.39, 127.00, 122.44, 122.40, 120.84, 120.47, 117.50, 115.32, 103.07, 52.77, 48.86, 46.39, 42.56, 39.11, 23.06, 18.79. C₂₉H₃₅N₇O, HRMS calculated for m/z [M+H]⁺: 498.2981 (calculated), 498.2990 (found).

### Example 83: 2-methyl-N-((R)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide

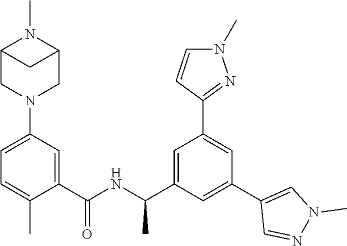 [SMILES: Cc1ccc(N2CC3CC(C2)N3C)cc1C(=O)N[C@H](C)c1cc(-c2cnn(C)c2)cc(-c2ccn(C)n2)c1]

(Method B) White solid, 77% yield. ¹H NMR (400 MHz, DMSO-d₆) δ 8.65 (d, J=8.2 Hz, 1H), 8.12 (s, 1H), 7.81 (s, 1H), 7.74 (s, 1H), 7.67 (d, J=2.3 Hz, 1H), 7.63 (s, 1H), 7.45 (s, 1H), 7.06 (dd, J=8.3, 5.2 Hz, 1H), 6.67 (q, J=7.0 Hz, 3H), 5.11 (p, J=7.1 Hz, 1H), 4.44-4.39 (m, 1H), 4.25 (d, J=6.3 Hz, 1H), 3.81 (d, J=1.9 Hz, 6H), 3.77-3.69 (m, 2H), 3.61 (ddd, J=21.3, 12.2, 6.3 Hz, 2H), 2.97 (d, J=5.0 Hz, 1H), 2.39 (d, J=3.3 Hz, 2H), 2.15 (s, 3H), 1.85 (d, J=10.2 Hz, 1H), 1.43 (d, J=7.0 Hz, 3H). ¹³C NMR (101 MHz, DMSO-d₆) δ 169.12, 150.53, 146.37, 146.34, 145.67, 144.95, 138.47, 138.33, 136.46, 134.47, 133.28, 132.72, 131.62, 128.39, 123.67, 123.51, 122.45, 122.39, 120.86, 120.46, 118.03, 115.11, 111.87, 109.98, 109.87, 103.07, 64.82, 62.78, 48.93, 48.84, 43.82, 39.11, 37.12, 29.39, 28.40, 23.06, 18.72, 18.69. C₃₀H₃₅N₇O, HRMS calculated for m/z [M+H]⁺: 510.2981 (calculated), 510.2989 (found).

### Example 84: N-(1-(4′-(2-(2-methoxyethoxy)ethoxy)-5-(1-methyl-1H-pyrazol-3-yl)-[1,1′-biphenyl]-3-yl)cyclopropyl)-2-methyl-5-(4-methylpiperazin-1-yl)benzamide

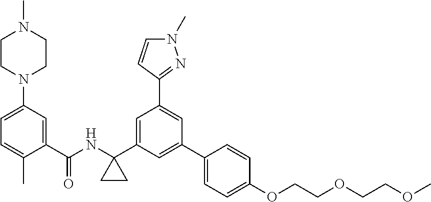 [SMILES: COCCOCCOc1ccc(-c2cc(-c3ccn(C)n3)cc(C3(NC(=O)c4cc(N5CCN(C)CC5)ccc4C)CC3)c2)cc1]

(Method A) White solid, 95% yield. ¹H NMR (400 MHz, DMSO-d₆) δ 8.93 (s, 1H), 7.72 (s, 1H), 7.67 (s, 1H), 7.59 (s, 1H), 7.56 (s, 1H), 7.54 (s, 1H), 7.29 (s, 1H), 7.07 (d, J=8.1 Hz, 1H), 6.98 (d, J=8.3 Hz, 2H), 6.92 (d, J=8.1 Hz, 2H), 6.68 (d, J=2.2 Hz, 1H), 4.22 (s, 2H), 4.07 (t, J=4.5 Hz, 2H), 3.82 (s, 3H), 3.69 (t, J=4.6 Hz, 2H), 3.53 (t, J=4.8 Hz, 2H), 3.18 (s, 3H), 3.09 (s, 2H), 2.90 (t, J=12.5 Hz, 2H), 2.80 (s, 3H), 2.20 (s, 3H), 1.31 (d, J=5.3 Hz, 2H), 1.26 (d, J=5.0 Hz, 2H). ¹³C NMR (101 MHz, DMSO-d₆) δ 170.16, 158.66, 150.56, 147.71, 144.92, 140.54, 138.07, 134.31, 133.34, 132.74, 131.73, 128.32, 127.21, 122.28, 120.94, 120.54, 117.65, 115.37, 115.27, 103.14, 71.78, 70.22, 69.43, 67.75, 58.54, 52.76, 46.35, 42.58, 39.12, 34.88, 18.87, 18.49. C₃₇H₄₅N₅O₄, HRMS calculated for m/z [M+H]⁺: 624.3550 (calculated), 624.3559 (found).

### Example 85: N-(1-(3,5-bis(1-methyl-1H-pyrazol-4-yl)phenyl)cyclopropyl)-2-methyl-5-(6-methyl-3,6-diazabicyclo[3.1.1]heptan-3-yl)benzamide

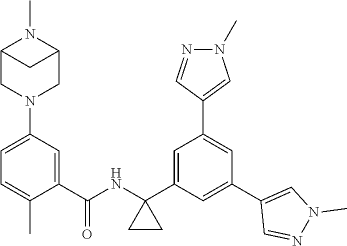 [SMILES: Cc1ccc(N2CC3CC(C2)N3C)cc1C(=O)NC1(c2cc(-c3cnn(C)c3)cc(-c3cnn(C)c3)c2)CC1]

(Method A) White solid, 79% yield. ¹H NMR (400 MHz, DMSO-d₆) δ 8.94 (s, 1H), 8.14 (s, 2H), 7.86 (d, J=2.0 Hz, 2H), 7.60 (d, J=1.7 Hz, 1H), 7.25 (s, 2H), 7.15 (t, J=7.2 Hz, 1H), 6.80-6.75 (m, 2H), 4.51 (d, J=6.0 Hz, 1H), 4.35 (d, J=6.2 Hz, 1H), 3.88 (s, 6H), 3.87-3.66 (m, 5H), 3.06 (d, J=5.0 Hz, 1H), 2.89 (dd, J=10.8, 5.9 Hz, 1H), 2.49 (s, 3H), 2.27 (s, 3H), 1.95 (d, J=10.3 Hz, 1H), 1.37 (d, J=5.0 Hz, 2H), 1.30 (d, J=4.8 Hz, 2H). ¹³C NMR (101 MHz, DMSO-d₆) δ 170.46, 145.74, 145.20, 145.03, 138.24, 138.10, 136.47, 136.44, 133.29, 131.74, 128.32, 123.74, 122.62, 119.76, 119.63, 112.04, 109.81, 64.87, 62.84, 48.96, 43.84, 39.12, 37.15, 34.81, 29.41, 18.72, 18.46. C₃₁H₃₅N₇O, HRMS calculated for m/z [M+H]⁺: 522.2981 (calculated), 522.2991 (found).

### Example 86: 2-methyl-N-(1-(3-(1-methyl-1H-pyrazol-3-yl)-5-(1-(oxetan-3-yl)-1H-pyrazol-4-yl)phenyl)cyclopropyl)-5-(4-methylpiperazin-1-yl)benzamide

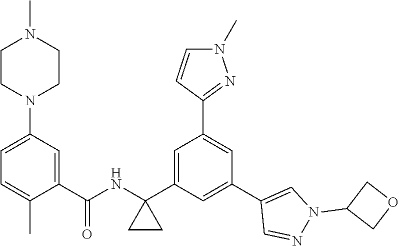 [SMILES: Cc1ccc(N2CCN(C)CC2)cc1C(=O)NC1(c2cc(-c3cnn(C4COC4)c3)cc(-c3ccn(C)n3)c2)CC1]

(Method A) White solid, 79% yield. ¹H NMR (400 MHz, DMSO-d₆) δ 8.90 (s, 1H), 8.38 (s, 1H), 7.97 (s, 1H), 7.73 (d, J=1.7 Hz, 1H), 7.67 (d, J=1.6 Hz, 1H), 7.59 (s, 1H), 7.21 (s, 1H), 7.07 (d, J=8.1 Hz, 1H), 6.92 (d, J=7.5 Hz, 2H), 6.65 (d, J=3.3 Hz, 1H), 5.56-5.49 (m, 1H), 4.92-4.85 (m, 4H), 3.81 (s, 3H), 3.76 (dd, J=22.0, 8.2 Hz, 4H), 3.47 (d, J=12.0 Hz, 2H), 2.90 (t, J=12.5 Hz, 2H), 2.80 (s, 3H), 2.21 (s, 3H), 1.29 (d, J=4.8 Hz, 2H), 1.24 (d, J=5.0 Hz, 2H). 13C NMR (101 MHz, DMSO-d₆) δ 170.14, 150.55, 147.70, 144.96, 138.11, 137.44, 134.18, 132.91, 132.67, 131.70, 127.23, 122.94, 120.53, 120.45, 119.86, 117.62, 115.33, 103.05, 77.11, 55.17, 52.77, 46.38, 42.59, 39.11, 34.83, 18.88, 18.50. C₃₂H₃₇N₇O₂, HRMS calculated for m/z [M+H]⁺: 552.3087 (calculated), 552.3093 (found).

### Example 87: N-(1-(3-(1-ethyl-1H-pyrazol-3-yl)-5-(1-(methoxymethyl)-1H-pyrazol-4-yl)phenyl)cyclopropyl)-2-methyl-5-(6-methyl-3,6-diazabicyclo[3.1.1]heptan-3-yl)benzamide

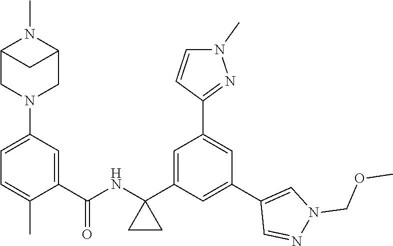 [SMILES: COCn1cc(-c2cc(-c3ccn(C)n3)cc(C3(NC(=O)c4cc(N5CC6CC(C5)N6C)ccc4C)CC3)c2)cn1]

(Method A) White solid, 87% yield. ¹H NMR (400 MHz, DMSO-d₆) δ 8.90 (s, 1H), 8.34 (s, 1H), 7.95 (s, 1H), 7.73 (s, 1H), 7.67 (d, J=2.1 Hz, 1H), 7.59 (d, J=3.3 Hz, 1H), 7.24 (d, J=2.4 Hz, 1H), 7.08 (t, J=7.3 Hz, 1H), 6.74-6.65 (m, 3H), 5.34 (s, 2H), 4.44 (d, J=6.0 Hz, 1H), 4.28 (d, J=6.3 Hz, 1H), 3.81 (s, 3H), 3.79-3.72 (m, 2H), 3.64 (dd, J=22.3, 12.2 Hz, 2H), 3.21 (s, 3H), 2.98 (d, J=5.0 Hz, 1H), 2.80 (dt, J=11.8, 6.5 Hz, 1H), 2.44-2.39 (m, 4H), 2.21 (s, 3H), 1.88 (d, J=10.1 Hz, 1H), 1.34-1.28 (m, 2H), 1.22 (d, J=6.2 Hz, 2H). ¹³C NMR (101 MHz, DMSO-d₆) δ 170.43, 150.52, 145.70, 145.03, 145.00, 138.28, 138.13, 137.85, 134.24, 132.77, 132.70, 131.74, 128.52, 123.85, 123.41, 120.76, 120.46, 119.94, 112.19, 112.04, 109.83, 103.09, 81.79, 64.87, 62.86, 56.62, 48.97, 43.85, 39.12, 37.15, 34.81, 29.43, 28.43, 26.16, 18.76, 18.74, 18.48. C₃₃H₃₉N₇O₂, HRMS calculated for m/z [M+H]⁺: 566.3243 (calculated), 566.3251 (found).

### Example 88: N-(1-(3-(1-(2-methoxyethyl)-1H-pyrazol-4-yl)-5-(1-methyl-1H-pyrazol-3-yl)phenyl)cyclopropyl)-2-methyl-5-(4-methylpiperazin-1-yl)benzamide

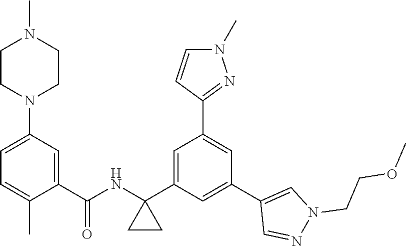 [SMILES: COCCn1cc(-c2cc(-c3ccn(C)n3)cc(C3(NC(=O)c4cc(N5CCN(C)CC5)ccc4C)CC3)c2)cn1]

(Method A) White solid, 87% yield. ¹H NMR (400 MHz, DMSO-d₆) δ 8.98 (s, 1H), 8.21 (s, 1H), 7.91 (s, 1H), 7.77 (s, 1H), 7.75 (d, J=2.2 Hz, 1H), 7.62 (d, J=4.6 Hz, 1H), 7.27 (s, 1H), 7.15 (d, J=8.1 Hz, 1H), 7.00 (d, J=8.0 Hz, 2H), 6.73 (d, J=2.3 Hz, 1H), 4.29 (t, J=5.3 Hz, 2H), 3.89 (s, 3H), 3.88-3.81 (m, 2H), 3.73 (t, J=5.3 Hz, 2H), 3.54 (d, J=12.0 Hz, 2H), 3.25 (s, 3H), 3.17 (t, J=14.4 Hz, 2H), 2.97 (t, J=12.5 Hz, 2H), 2.88 (s, 3H), 2.29 (s, 3H), 1.36 (d, J=5.1 Hz, 2H), 1.34-1.28 (m, 2H). ¹³C NMR (101 MHz, DMSO-d₆) δ 170.13, 150.58, 147.71, 144.89, 138.11, 136.62, 134.17, 133.18, 132.67, 131.71, 128.19, 127.20, 122.32, 120.55, 120.14, 119.70, 117.61, 115.31, 103.05, 70.95, 58.44, 52.75, 51.78, 46.37, 42.58, 39.11, 34.83, 18.89, 18.44. C₃₂H₃₉N₇O₂, HRMS calculated for m/z [M+H]⁺: 553.3165 (calculated), 553.3172 (found).

### Example 89: FRET-Based PL^{pro} Enzymatic Assay

The SARS-CoV-2 PL^{pro} enzymnatic assays were carried out as follows: The assay was assembled in 96-well plates with 100 μl of 200 nM PL^{pro} protein in PL^{pro} reaction buffer [50 mM Hepes (pH 7.5), 0.01% Triton X-100, and 5 mM DTT], Then, 1 μL of testing compound at various concentrations was added to each well and incubated a 30° C. for 30 minutes. The enzymatic reaction was initiated by adding 1 μl of 1 mM FRET substrate (the final substrate concentration is 10 μM). The reaction was monitored in a Cytation 5 image reader with filters for excitation at 360/40 nm and emission at 460/40 nm at 30° C. for 1 hour. The initial velocity of the enzymatic reaction with and without testing compounds was calculated by linear regression for the first 15 minutes of the kinetic progress curve. The IC₅₀ values were calculated by plotting the initial velocity against various concentrations of testing compounds with a dose-response function in Prism 8 software (Table 2).

TABLE 2
PL^{pro} enzymatic assay data

| Cmpd | IC₅₀ (μM) |
|---|---|
| 1 | 0.14 ± 0.01 |
| 2 | 0.19 ± 0.02 |
| 3 | 0.23 ± 0.02 |
| 4 | 0.27 ± 0.01 |
| 5 | 0.39 ± 0.04 |
| 6 | 0.40 ± 0.04 |
| 7 | 0.62 ± 0.13 |
| 8 | 0.18 ± 0.01 |
| 9 | 0.27 ± 0.02 |
| 10 | 0.20 ± 0.01 |
| 11 | 0.15 ± 0.01 |
| 12 | 0.11 ± 0.01 |
| 13 | 0.65 ± 0.06 |
| 14 | 0.12 ± 0.01 |
| 15 | 0.20 ± 0.01 |
| 16 | 0.15 ± 0.01 |
| 17 | 0.20 ± 0.01 |
| 18 | 0.29 ± 0.02 |
| 19 | 5.23 ± 0.28 |
| 20 | 0.16 ± 0.04 |
| 21 | 1.30 ± 0.11 |
| 22 | 0.18 ± 0.02 |
| 23 | 0.11 ± 0.01 |
| 24 | 0.14 ± 0.02 |
| 25 | 0.10 ± 0.06 |
| 26 | 0.12 ± 0.01 |
| 27 | 0.12 ± 0.01 |
| 28 | 0.08 ± 0.01 |
| 29 | 0.12 ± 0.01 |
| 30 | 0.12 ± 0.01 |
| 31 | 0.86 ± 0.05 |
| 32 | 0.76 ± 0.05 |
| 33 | 0.53 ± 0.06 |
| 34 | 0.48 ± 0.04 |
| 35 | 0.33 ± 0.04 |
| 36 | 0.17 ± 0.01 |
| 37 | 0.16 ± 0.01 |
| 38 | 0.12 ± 0.01 |
| 39 | 0.12 ± 0.01 |
| 40 | 0.11 ± 0.01 |
| 41 | 0.11 ± 0.01 |
| 42 | 0.21 ± 0.01 |
| 43 | 0.21 ± 0.01 |
| 44 | 0.11 ± 0.01 |
| 45 | 0.08 ± 0.01 |
| 46 | 0.10 ± 0.01 |
| 47 | 0.25 ± 0.02 |
| 48 | 0.10 ± 0.01 |
| 49 | 0.10 ± 0.01 |
| 50 | 0.11 ± 0.01 |
| 51 | 0.08 ± 0.01 |
| 52 | 0.10 ± 0.01 |
| 53 | 0.09 ± 0.01 |
| 54 | 0.11 ± 0.01 |
| 55 | 0.11 ± 0.01 |
| 56 | 0.12 ± 0.01 |
| 57 | 0.13 ± 0.01 |
| 58 | 0.13 ± 0.01 |
| 59 | 0.19 ± 0.01 |
| 60 | 0.10 ± 0.02 |
| 61 | 0.10 ± 0.01 |
| 62 | 0.14 ± 0.01 |
| 63 | 0.13 ± 0.01 |
| 64 | 0.10 ± 0.01 |
| 65 | 0.10 ± 0.01 |
| 66 | 0.10 ± 0.01 |
| 67 | 0.11 ± 0.01 |
| 68 | 0.10 ± 0.01 |
| 69 | 0.11 ± 0.01 |
| 70 | 0.16 ± 0.01 |
| 71 | 0.15 ± 0.01 |
| 72 | 0.13 ± 0.01 |
| 73 | 2.53 ± 0.15 |
| 74 | 0.23 ± 0.02 |
| 75 | 0.14 ± 0.01 |
| 76 | 0.11 ± 0.01 |
| 77 | 0.08 ± 0.01 |
| 78 | 0.08 ± 0.01 |
| 79 | 0.14 ± 0.01 |
| 80 | 0.14 ± 0.01 |
| 81 | 0.31 ± 0.01 |
| 82 | 0.13 ± 0.01 |
| 83 | 0.13 ± 0.01 |
| 84 | 0.10 ± 0.01 |
| 85 | 0.14 ± 0.01 |
| 86 | 0.15 ± 0.01 |
| 87 | 0.13 ± 0.01 |
| 88 | 0.10 ± 0.01 |

### Enumerated Embodiments

The following exemplary embodiments are provided, the numbering of which is not to be construed as designating levels of importance:

Embodiment 1 provides a compound of Formula (I), or a salt, stereoisomer, or isotopically labeled derivative thereof:

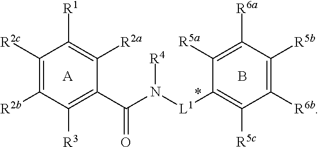

wherein:
R¹ is selected from the group consisting of

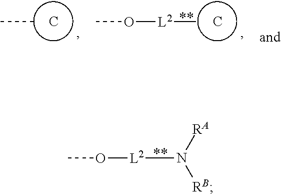

R^{2a}, R^{2b}, and R^{2c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R³ is CH₃;
R⁴ is H;
R^{5a}, R^{5b}, and R^{5c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{6a} is selected from the group consisting of

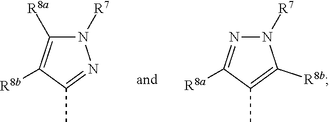

R^{6b} is selected from the group consisting of

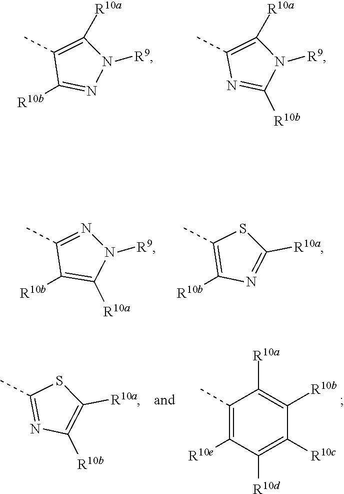

ring C is selected from the group consisting of

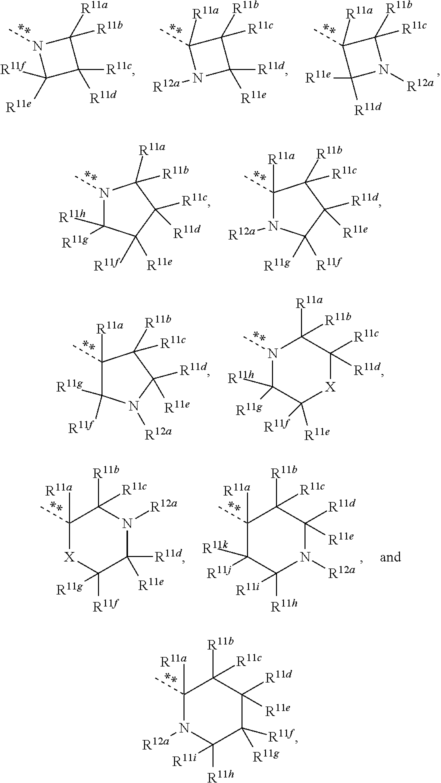

wherein ** indicates a bond between L² and ring C or N(R^{A})(R^{B});
L¹ is selected from the group consisting of

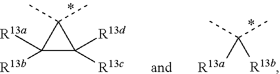

wherein * indicates a bond between L¹ and ring B;
L² is selected from the group consisting of -(optionally substituted C₁-C₆ alkylenyl)- and -(optionally substituted C₂-C₆ heteroalkylenyl)-;
R⁷ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{8a} and R^{8b} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R⁹ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{10a}, R^{10b}, R^{10c}, R^{10d}, and R^{10e}, if present, are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{1a}, R^{11b}, R^{11c}, R^{11d}, R^{11e}, R^{11f}, R^{11g}, R^{11h}, R¹¹ⁱ, and R^{11j}, if present, are each independently selected from the group consisting of H, halogen, optionally substituted C₁-C₆ alkyl, and N(R^{A})(R^{B}), or
   two substituents selected from the group consisting of R^{11a}, R^{11b}, R^{11c}, R^{11d}, R^{11e}, R^{11f}, R^{11g}, R^{11h}, R¹¹ⁱ, and R^{11j} can combine with the atoms to which they are bound to form an optionally substituted C₃-C₈ heterocycloalkyl or optionally substituted C₃-C₈ cycloalkyl;
R^{12a} and R^{12b}, if present, are each independently selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{13a}, R^{13b}, R^{13c}, and R^{13d}, if present, are each independently selected from the group consisting of H and optionally substituted C₁-C₆ alkyl;
X is selected from the group consisting of —C(R¹¹ⁱ)(R^{11j})—, —N(R^{12b})—, —O—, and —S—;
each occurrence of R^{A} and R^{B}, if present, is independently selected from the group consisting of H, C(═O)R^{C}, C(═O)OR^{C}, C(═O)N(R^{C})(R^{D}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
R^{C} and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
wherein the compound is not 2-methyl-N-(1-(3-(1-methyl-1H-pyrazol-4-yl)-5-(thiazol-5-yl)phenyl)cyclopropyl)-5-(piperazin-1-yl)benzamide.

Embodiment 2 provides the compound of Embodiment 1, wherein L¹ is

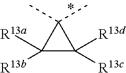

### and R^{6b} is

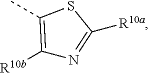

and at least one of the following applies:
(a) no more than one of R^{10a} and R^{10b} is H, optionally wherein at least one of R^{10a} and R^{10b} is C₁-C₆ alkyl;
(b) R^{6a} is

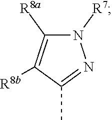

and
(c) R^{6a} is

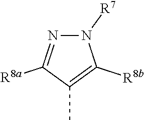

and R⁷ is C₂-C₆ alkyl.

Embodiment 3 provides the compound of Embodiment 1 or 2, wherein L¹ is

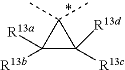

and one of the following applies:
(a) R¹ is

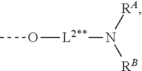

R^{6a} is

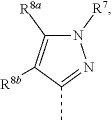

and R^{6b} is selected from the group consisting of consisting of R

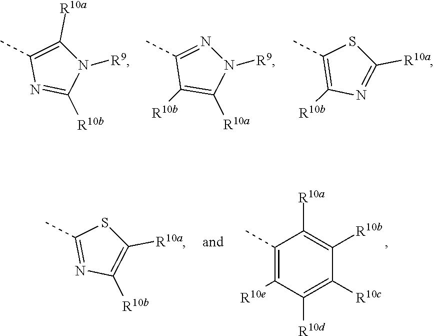

(b) R¹ is

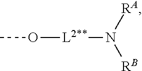

R^{6a} is

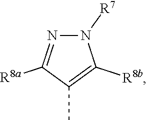

and R^{6b} is selected from the group consisting of

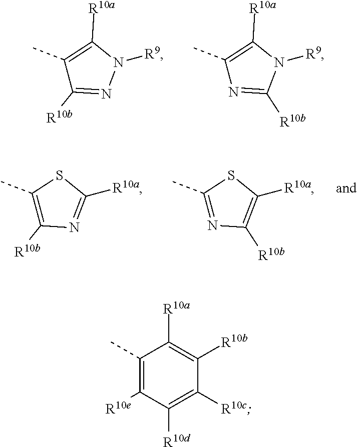

(c) R¹ is

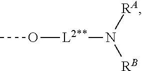

R^{6a} is

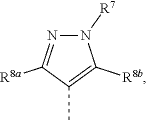

and N(R^{A})(R^{B}) is NEt₂;
(d) R¹ is

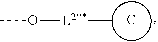

ring C is

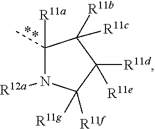

and at least one of the following applies: (i) at least one of R^{11a}, R^{11b}, R^{11c}, R^{11d}, R^{11e}, R^{11f}, R^{11g} is F, or (ii) the carbon atom substituted with R^{11a} has (S)-configuration;
(e) R¹ is

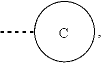

ring C is

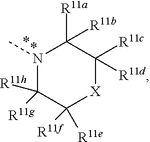

X is N(R^{12b}), and at least one of the following applies: (i) one of R^{2a}, R^{2b}, and R^{2c} is F, (ii) R^{12b} is C₂-C₆ alkyl or C₃-C₈ cycloalkyl, (iii) no more than three of R^{11c}, R^{11d}, R^{11e}, and R^{11f} are H, (iv) at least one of R⁷ or R⁹ is substituted C₁-C₆ alkyl, optionally substituted C₃-C₈ cycloalkyl, or optionally substituted C₂-C₈ heterocycloalkyl, (v) R^{6b} is

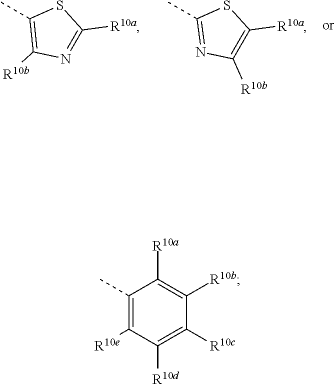

or (vi) R⁷ and R⁹ are both CH₃ or CH₂CH₃;
(f) R¹ is

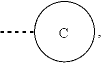

ring C is

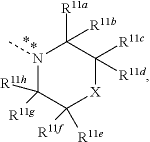

X is N(R^{12b}), one of R^{11a}, R^{11b}, R^{11h} and R^{11g} is CH₃, and at least one of the following applies: (i) R⁷ and R⁹ are both
CH₃, or (ii) R^{6a} is

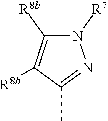

and R^{6b} is selected from the group consisting of

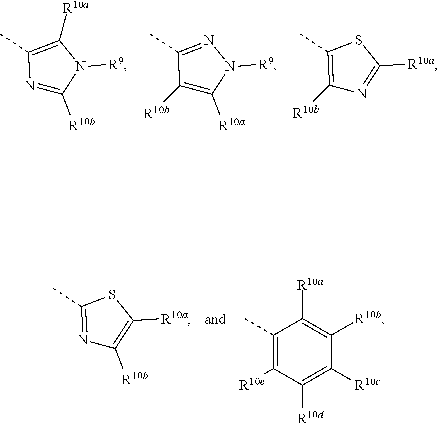

or (iii) R^{6a} is

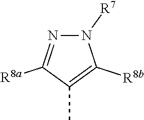

and R^{6b} is selected from the group consisting of

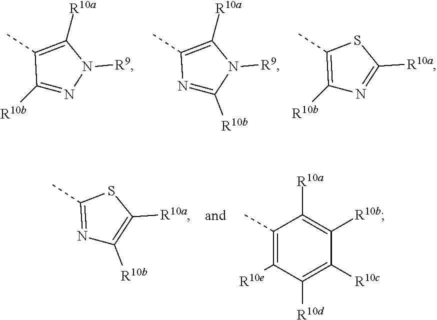

(g) R¹ is

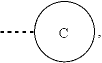

ring C is

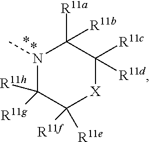

X is N(R^{12b}), one substituent selected from the group consisting of R^{11a} and R^{11b} combines with one substituent selected from the group consisting of R^{11e} and R^{11f} to form a C₃-C₅ heterocycloalkyl; and
(h) R¹ is

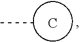

ring C is

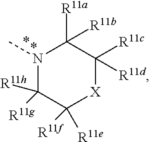

X is N(R^{12b}), one substituent selected from the group consisting of R^{11a}, R^{11b}, R^{11c}, and R^{11d} combines with one substituent selected from the group consisting of R^{11e}, R^{11f}, R^{11g} and R^{11h} to form a C₄-C₈ heterocycloalkyl, and R⁷ and R⁹ are both CH₃.

Embodiment 4 provides the compound of any one of Embodiments 1-3, which is selected from the group consisting of:

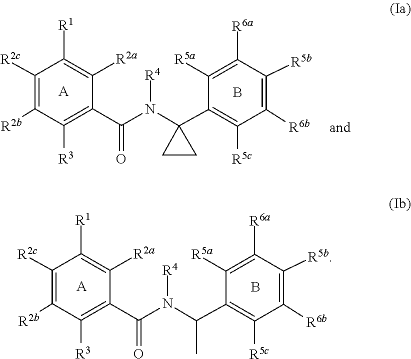

Embodiment 5 provides the compound of any one of Embodiments 1-4, which is selected from the group consisting of:

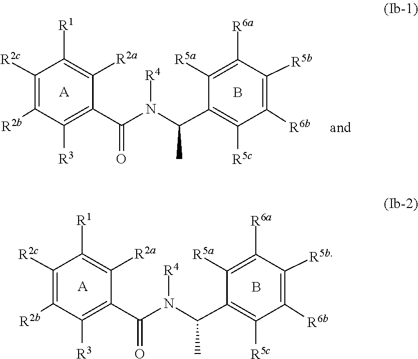

Embodiment 6 provides the compound of any one of Embodiments 1-5, wherein at least one of the following applies:
(a) R^{11a}, R^{11b}, R^{11e}, R^{11d}, R^{11e}, R^{11f}, R^{11g}, R^{11h}h, R¹¹ⁱ, and R^{11j}, if present, are each independently selected from the group consisting of H, F, and CH₃;
(b) two substituents selected from the group consisting of R^{11a}, R^{11b}, R^{11c}, R^{11d}, R^{11e}, R^{11f}, R^{11g}, R^{11h}, R¹¹ⁱ, and R^{11j}, combine with the atoms to which they are bound to form an optionally substituted azetidine or optionally substituted pyrrolidine; and (c) R^{12a} and R^{12b}, if present, are each independently selected from the group consisting of H, CH₃, CH(CH₃)₂, C(CH₃)₃, cyclopropyl, and cyclobutyl.

Embodiment 7 provides the compound of any one of Embodiments 1-6, wherein ring C is selected from the group consisting of

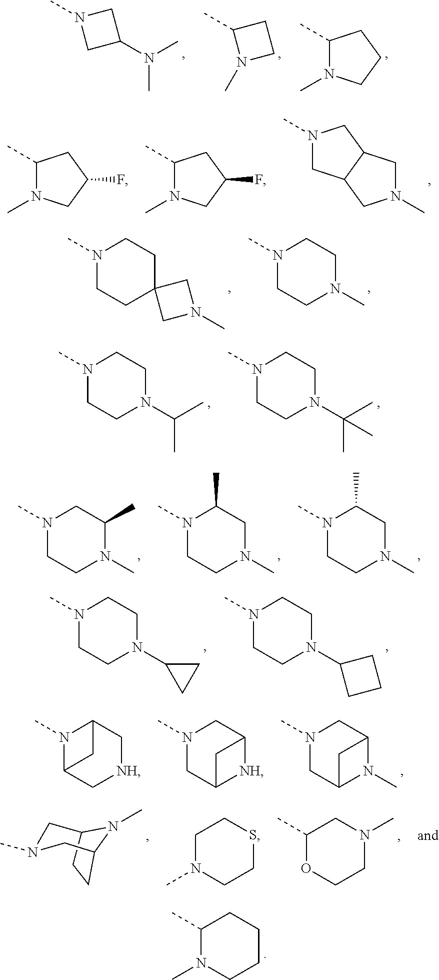

Embodiment 8 provides the compound of any one of Embodiments 1-7, wherein R¹ is selected from the group consisting of

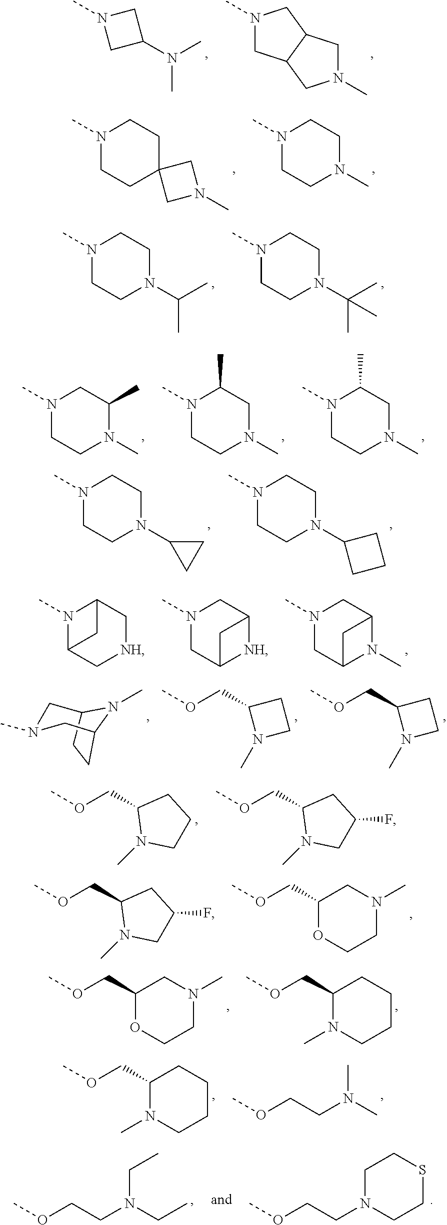

Embodiment 9 provides the compound of any one of Embodiments 1-8, wherein R^{2a}, R^{2b}, and R^{2c} are each independently selected from the group consisting of H and F.

Embodiment 10 provides the compound of any one of Embodiments 1-9, wherein R^{5a}, R^{5b}, and R^{5c} are each independently H.

Embodiment 11 provides the compound of any one of Embodiments 1-10, wherein R⁷ is selected from the group consisting of CH₃, CF₂H, CH₂CH₃, CH(CH₃)₂, and oxetanyl.

Embodiment 12 provides the compound of any one of Embodiments 1-11, wherein R^{8a} and R^{8b} are each independently H.

Embodiment 13 provides the compound of any one of Embodiments 1-12, wherein R^{6a} is selected from the group consisting of

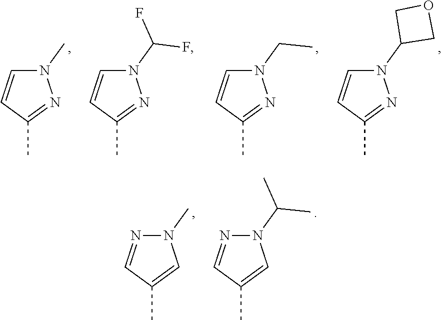

Embodiment 14 provides the compound of any one of Embodiments 1-13, wherein R⁹ is selected from the group consisting of CH₃, CH₂CH₃, CF₂H, CH₂CN, CH₂OCH₃, CH₂CF₂H, CH₂CF₃, CH₂(cyclopropyl), (CH₂)₂F, (CH₂)₂CN, (CH₂)₂OH, (CH₂)₂OCH₃, cyclopropyl, and oxetanyl.

Embodiment 15 provides the compound of any one of Embodiments 1-14, wherein R^{10a}, R^{10b}, R^{10c}, R^{10d}, and R^{11e}, if present, are each independently selected from the group consisting of H, CH₃, and O(CH₂)₂O(CH₂)₂OCH₃.

Embodiment 16 provides the compound of any one of Embodiments 1-15, wherein R^{6b} is selected from the group consisting of

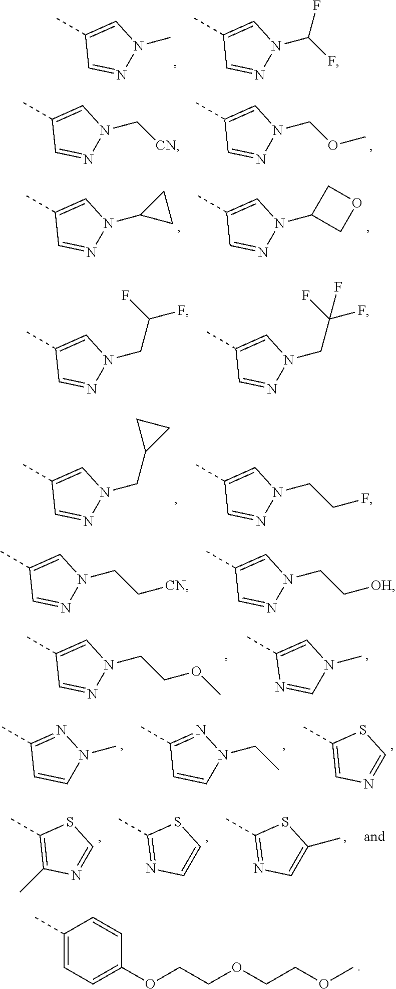

Embodiment 17 provides the compound of any one of Embodiments 1-16, which is selected from the group consisting of:
N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
5-(3-(dimethylamino)azetidin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-5-((4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4S)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4R)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2R,4R)-4-fluoro-1-methylpyrrolidin-2-yl)methoxy)-2-methylbenzamide;
N-(1-(3-(1-ethyl-1H-pyrazol-3-yl)-5-(1-methyl-H-pyrazol-4-yl)phenyl)cyclopropyl)-5-(((2R,4S)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-2-methyl-5-(2-methyl-2,7-diazaspiro[3.5]nonan-7-yl)benzamide;
N-(1-(3-(1-ethyl-1H-pyrazol-3-yl)-5-(1-methyl-1H-pyrazol-4-yl)phenyl)cyclopropyl)-2-methyl-5-(5-methylhexahydropyrrolo[3,4-c]pyrrol-2(1H)-yl)benzamide;
4-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-4-fluoro-2-methyl-5-(4-methylpiperazin-1-yl)benzamide;
3-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-3-fluoro-2-methyl-5-(4-methylpiperazin-1-yl)benzamide;
5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-fluoro-6-methyl-3-(4-methylpiperazin-1-yl)benzamide;
5-(4-(tert-butyl)piperazin-1-yl)-N-(1-(3-(1-ethyl-1H-pyrazol-3-yl)-5-(1-methyl-1H-pyrazol-4-yl)phenyl)cyclopropyl)-2-methylbenzamide;
5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
N-(1-(3-(1-(cyclopropylmethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-ethyl-1H-pyrazol-3-yl)phenyl)cyclopropyl)-2-methyl-5-(4-methylpiperazin-1-yl)benzamide;
N-(1-(3-(1-(2,2-difluoroethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-cyclopropyl-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2,2,2-trifluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(thiazol-5-yl)phenyl)cyclopropyl)-2-methyl-5-(4-methylpiperazin-1-yl)benzamide;
5-(4-cyclobutylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-(2-thiomorpholinoethoxy)benzamide;
N-(1-(3,5-bis(1-ethyl-1H-pyrazol-3-yl)phenyl)cyclopropyl)-2-methyl-5-((1-methylazetidin-2-yl)methoxy)benzamide;
(S)-N-(1-(3,5-bis(1-ethyl-1H-pyrazol-3-yl)phenyl)cyclopropyl)-2-methyl-5-((1-methylazetidin-2-yl)methoxy)benzamide;
(R)-N-(1-(3,5-bis(1-ethyl-1H-pyrazol-3-yl)phenyl)cyclopropyl)-2-methyl-5-((1-methylazetidin-2-yl)methoxy)benzamide;
N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(2-fluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(4-methylthiazol-5-yl)phenyl)cyclopropyl)-2-methyl-5-(4-methylpiperazin-1-yl)benzamide;
N-(1-(3-(1-ethyl-1H-pyrazol-3-yl)-5-(thiazol-2-yl)phenyl)cyclopropyl)-2-methyl-5-(4-methylpiperazin-1-yl)benzamide;
N-(1-(3-(1-ethyl-1H-pyrazol-3-yl)-5-(5-methylthiazol-2-yl)phenyl)cyclopropyl)-2-methyl-5-(4-methylpiperazin-1-yl)benzamide;
5-(2-(dimethylamino)ethoxy)-N-(1-(3-(1-ethyl-1H-pyrazol-3-yl)-5-(thiazol-5-yl)phenyl)cyclopropyl)-2-methylbenzamide;
5-(2-(dimethylamino)ethoxy)-N-(1-(3-(1-ethyl-1H-pyrazol-3-yl)-5-(thiazol-2-yl)phenyl)cyclopropyl)-2-methylbenzamide;
N-(1-(3-(1-(2-cyanoethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2-hydroxyethyl)-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(1-methyl-1H-pyrazol-4-yl)phenyl)cyclopropyl)-5-(4-methylpiperazin-1-yl)benzamide;
5-(2-(diethylamino)ethoxy)-N-(1-(3-(1-ethyl-1H-pyrazol-3-yl)-5-(1-methyl-1H-pyrazol-4-yl)phenyl)cyclopropyl)-2-methylbenzamide;
5-(3-(dimethylamino)azetidin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-ethyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(4-methylpiperazin-1-yl)benzamide;
5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(R)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(S)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
N-(1-(3-(1-ethyl-1H-pyrazol-3-yl)-5-(1-(oxetan-3-yl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((S)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((R)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-(1-(3-(1-ethyl-1H-pyrazol-3-yl)-5-(1-(2-methoxyethyl)-1H-pyrazol-4-yl)phenyl)cyclopropyl)-2-methyl-5-(4-methylpiperazin-1-yl)benzamide;
N-(1-(3-(1-(cyanomethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-methyl-1H-pyrazol-4-yl)phenyl)cyclopropyl)-2-methyl-5-(4-methylpiperazin-1-yl)benzamide;
N-(1-(3-(1-isopropyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-imidazol-4-yl)phenyl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-5-yl)phenyl)cyclopropyl)-5-(4-methylpiperazin-1-yl)benzamide;
5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(R)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(S)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
N-(1-(3-(1-(2-fluoroethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-2-yl)phenyl)cyclopropyl)-5-(4-methylpiperazin-1-yl)benzamide;
5-(3,6-diazabicyclo[3.1.1]heptan-3-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)cyclopropyl)-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-4-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-4-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
N-(1-(3,5-bis(1-methyl-1H-pyrazol-4-yl)phenyl)cyclopropyl)-2-methyl-5-((1-methylazetidin-2-yl)methoxy)benzamide;
(S)-N-(1-(3,5-bis(1-methyl-1H-pyrazol-4-yl)phenyl)cyclopropyl)-2-methyl-5-((1-methylazetidin-2-yl)methoxy)benzamide;
(R)-N-(1-(3,5-bis(1-methyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(oxetan-3-yl)-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-2-methyl-5-(3-methyl-3,6-diazabicyclo[3.1.1]heptan-6-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
N-(1-(3-(1-ethyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-(methoxymethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-((R)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((R)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1R,5S)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(5-methylthiazol-2-yl)phenyl)cyclopropyl)-5-(4-methylpiperazin-1-yl)benzamide;
2-methyl-N-(1-(3-(1-methyl-1H-pyrazol-3-yl)-5-(4-methylthiazol-5-yl)phenyl)cyclopropyl)-5-(4-methylpiperazin-1-yl)benzamide;
5-(3,6-diazabicyclo[3.1.1]heptan-6-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(R)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(S)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
2-methyl-N-(1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((R)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((S)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
N-(1-(4′-(2-(2-methoxyethoxy)ethoxy)-5-(1-methyl-1H-pyrazol-3-yl)-[1,1′-biphenyl]-3-yl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-(oxetan-3-yl)-1H-pyrazol-4-yl)phenyl)cyclopropyl)-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(6-methyl-3,6-diazabicyclo[3.1.1]heptan-3-yl)benzamide; and
N-(1-(3-(1-(2-methoxyethyl)-1H-pyrazol-4-yl)-5-(1-methyl-1H-pyrazol-3-yl)phenyl)cyclopropyl)-2-methyl-5-(4-methylpiperazin-1-yl)benzamide.

Embodiment 18 provides a pharmaceutical composition comprising at least one compound of any one of Embodiments 1-17 and a pharmaceutically acceptable carrier.

Embodiment 19 provides the pharmaceutical composition of Embodiment 18, further comprising at least one antiviral agent.

Embodiment 20 provides the pharmaceutical composition of Embodiment 19, wherein the antiviral agent is a protease inhibitor.

Embodiment 21 provides the pharmaceutical composition of Embodiment 19 or 20, wherein the antiviral agent is selected from the group consisting of nirmatrelvir, ensiltrelvir, molnupiravir, VV116, and remdesivir.

Embodiment 22 provides a method for promoting an antiviral effect in a subject, the method comprising administering at least one compound of any one of Embodiments 1-17 and/or the pharmaceutical composition of any one of Embodiments 18-20.

Embodiment 23 provides the method of Embodiment 22, wherein the method further comprises administering to the subject at least one antiviral agent.

Embodiment 24 provides the method of Embodiment 23, wherein the antiviral agent is a protease inhibitor.

Embodiment 25 provides the method of Embodiment 23, wherein the antiviral agent is selected from the group consisting of nirmatrelvir, ensiltrelvir, molnupiravir, VV116, and remdesivir.

Embodiment 26 provides a method for inhibiting a papain-like protease in a subject, the method comprising administering to the subject at least one compound of any one of Embodiments 1-17 and/or the pharmaceutical composition of any one of Embodiments 18-20.

Embodiment 27 provides a method for treating, preventing, and/or ameliorating a viral infection in a subject, the method comprising administering to the subject at least one compound of any one of Embodiments 1-17 and/or the pharmaceutical composition of any one of Embodiments 18-20.

Embodiment 28 provides the method of Embodiment 27, wherein the viral infection is caused by a coronavirus.

Embodiment 29 provides the method of Embodiment 28, wherein the coronavirus is SARS-CoV-2.

Embodiment 30 provides the method of any one of Embodiments 27-29, wherein the viral infection is COVID-19.

Embodiment 31 provides the method of any one of Embodiments 22-30, wherein the subject is a mammal.

Embodiment 32 provides the method of Embodiment 31, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound of Formula (I), or a salt, stereoisomer, or isotopically labeled derivative thereof: 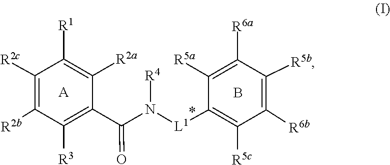
wherein:
R¹ is selected from the group consisting of 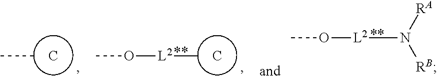
R^{2a}, R^{2b}, and R^{2c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R³ is CH₃;
R⁴ is H;
R^{5a}, R^{5b}, and R^{5c} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{6a} is selected from the group consisting of 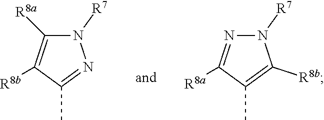
R^{6b} is selected from the group consisting of 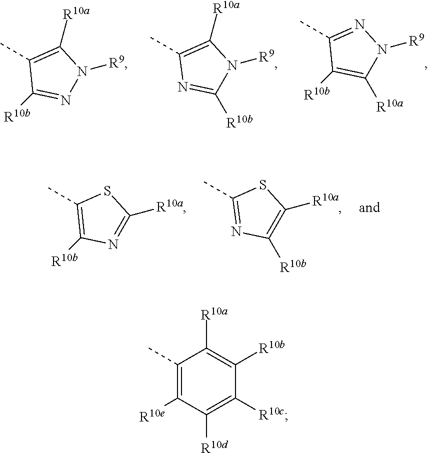
ring C is selected from the group consisting of 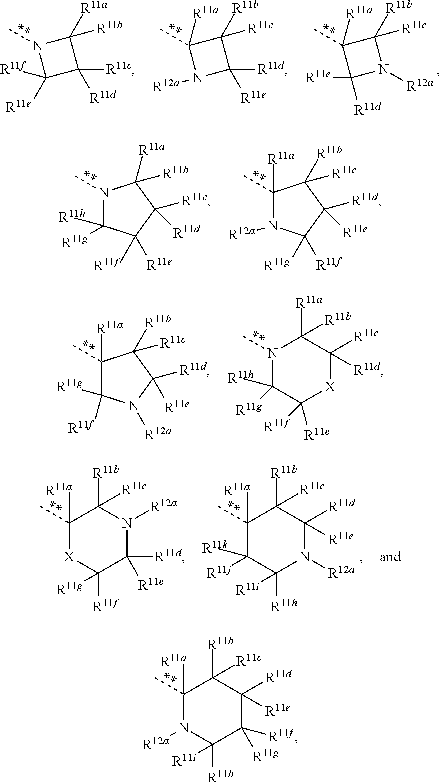
wherein ** indicates a bond between L² and ring C or N(R^{A})(R^{B});
L¹ is selected from the group consisting of 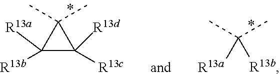
wherein * indicates a bond between L¹ and ring B;
L² is selected from the group consisting of -(optionally substituted C₁-C₆ alkylenyl)- and -(optionally substituted C₂-C₆ heteroalkylenyl)-;
R⁷ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B})
R^{8a} and R^{8b} are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}) CN, and NO₂;
R⁹ is selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{10a}, R^{10b}, R^{10c}, R^{10d}, and R^{10e}, if present, are each independently selected from the group consisting of H, halogen, optionally substituted C₁-C₆ alkyl, OR^{A}, N(R^{A})(R^{B}), C(═O)R^{A}, C(═O)OR^{A}, C(═O)N(R^{A})(R^{B}), CN, and NO₂;
R^{11a}, R^{11b}, R^{11c}, R^{11d}, R^{11e}, R^{11f}, R^{11g}, R^{11h}, R¹¹ⁱ, and R^{11j}, if present, are each independently selected from the group consisting of H, halogen, optionally substituted C₁-C₆ alkyl, and N(R^{A})(R^{B}), or
two substituents selected from the group consisting of R^{11a}, R^{11b}, R^{11c}, R^{11d}, R^{11e}, R^{11f}, R^{11g}, R^{11h}, R¹¹ⁱ, and R^{11j} can combine with the atoms to which they are bound to form an optionally substituted C₃-C₈ heterocycloalkyl or optionally substituted C₃-C₈ cycloalkyl;
R^{12a} and R^{12b}, if present, are each independently selected from the group consisting of H, optionally substituted C₁-C₆ alkyl, optionally substituted C₃-C₈ cycloalkyl, optionally substituted C₂-C₈ heterocycloalkyl, OR^{A}, N(R^{A})(R^{B}), C(═O)R^{A}, C(═O)OR^{A}, and C(═O)N(R^{A})(R^{B});
R^{13a}, R^{13b}, R^{13c}, and R^{13d}, if present, are each independently selected from the group consisting of H and optionally substituted C₁-C₆ alkyl;
X is selected from the group consisting of —C(R¹¹ⁱ)(R^{11j})—, —N(R^{12b})—, —O—, and —S—;
each occurrence of R^{A} and R^{B}, if present, is independently selected from the group consisting of H, C(═O)R^{C}, C(═O)OR^{C}, C(═O)N(R^{C})(R^{D}), optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl;
R^{C} and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl; and
wherein the compound is not 2-methyl-N-(1-(3-(1-methyl-1H-pyrazol-4-yl)-5-(thiazol-5-yl)phenyl)cyclopropyl)-5-(piperazin-1-yl)benzamide.

2. The compound of claim 1, wherein L¹ is 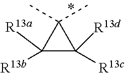
and R^{6b} is 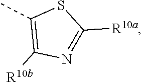
and at least one of the following applies:
(a) no more than one of R^{10a} and R^{10b} is H, optionally wherein at least one of R^{10a} and R^{10b} b is C₁-C₆ alkyl;
(b) R^{6a} is 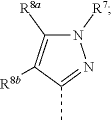
and
(c) R^{6a} is 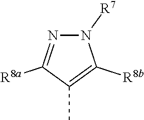
and R⁷ is C₂-C₆ alkyl.

3. The compound of claim 1, wherein L¹ is 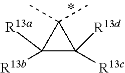
and one of the following applies:
(a) R¹ is 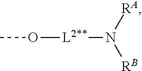
, R^{6a} is 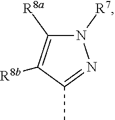
and R^{6b} is selected from the group consisting of consisting of 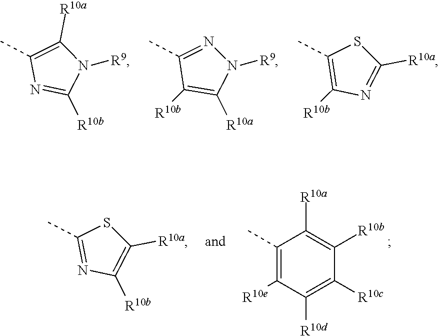
(b) R¹ is 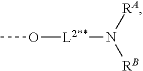
R^{6a} is 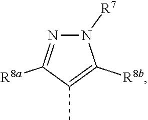
and R^{6b} is selected from the group consisting of 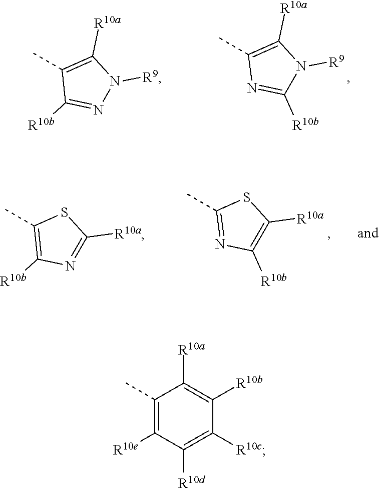
(c) R^{t} is 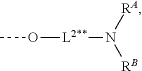
R^{6a} is 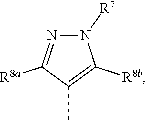
and N(R^{A})(R^{B}) is NEt₂;
(d) R¹ is 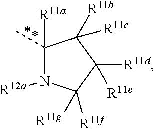
ring C is 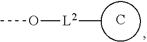
and at least one of the following applies: (i) at least one of R^{11a}, R^{11b}, R^{11c}, R^{11d}, R^{11e}, R^{11f}, or R^{11g} is F, or (ii) the carbon atom substituted with R^{11a} has (S)-configuration;
(e) R¹ is 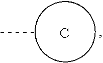
ring C is 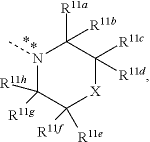
X is N(R^{12b}), and at least one of the following applies: (i) one of R^{2a}, R^{2b}, and R^{2c} is F, (ii) R^{12b} is C₂-C₆ alkyl or C₃-C₈ cycloalkyl, (iii) no more than three of R^{11c}, R^{11d}, R^{11e}, and R^{11f} are H, (iv) at least one of R⁷ or R⁹ is substituted C₁-C₆ alkyl, optionally substituted C₃-C₈ cycloalkyl, or optionally substituted C₂-C₈ heterocycloalkyl, (v) R^{6b} is 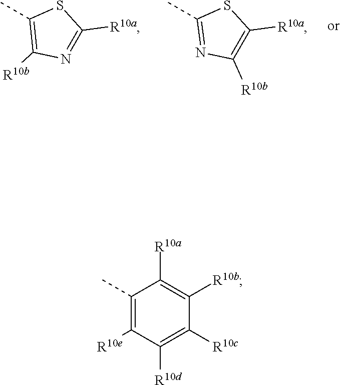
or (vi) R⁷ and R⁹ are both CH₃ or CH₂CH₃;
(f) R is 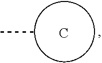
ring C is 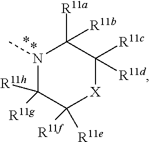
X is N(R^{12b}), one of R^{11a}, R^{11b}, R^{11h}, and R^{11g} is CH₃, and at least one of the following applies: (i) R⁷ and R⁹ are both CH₃, or (ii) R^{6a} is 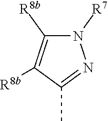
and R^{6b} is selected from the group consisting of 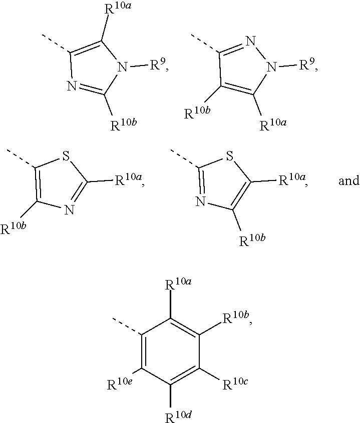
or (iii) R^{6a} is 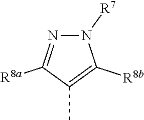
and R^{6b} is selected from the group consisting of 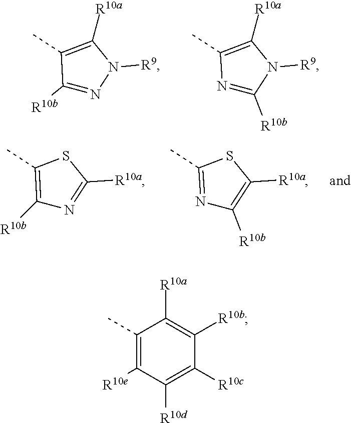
(g) R¹ is 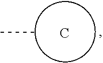
ring C is 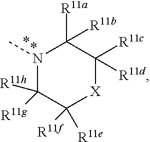
X is N(R^{12b}), one substituent selected from the group consisting of R^{11a} and R^{11b} combines with one substituent selected from the group consisting of R^{11e} and R^{11f} to form a C₃-C₅ heterocycloalkyl; and
(h) R¹ is 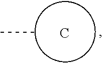
ring C is 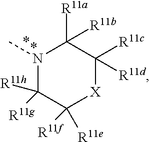
X is N(R^{12b}), one substituent selected from the group consisting of R^{11a}, R^{11b}, R^{11c}, and R^{11d} combines with one substituent selected from the group consisting of R^{11e}, R^{11f}, R^{11g} and R^{11h} to form a C₄-C₈ heterocycloalkyl, and R⁷ and R⁹ are both CH₃.

4. The compound of claim 1, which is selected from the group consisting of:

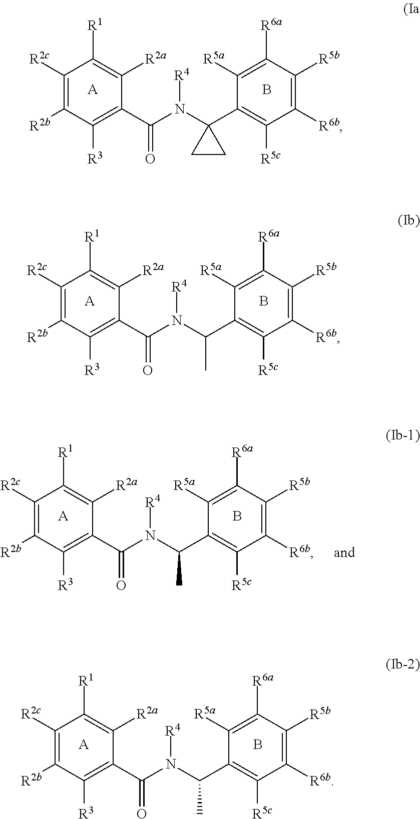

5. The compound of claim 1, wherein at least one of the following applies:
(c) R^{11a}, R^{11b}, R^{11c}, R^{11d}, R^{11e}, R^{11f}, R^{11g}, R^{11h}, R¹¹ⁱ, and R^{11j}, if present, are each independently selected from the group consisting of H, F, and CH₃;
(d) two substituents selected from the group consisting of R^{11a}, R^{11b}, R^{11e}, R^{11d}, R^{11e}, R^{11f}, R^{11g}, R^{11h}, R¹¹ⁱ, and R^{11j}, combine with the atoms to which they are bound to form an optionally substituted azetidine or optionally substituted pyrrolidine; and
(c) R^{12a} and R^{12b}, if present, are each independently selected from the group consisting of H, CH₃, CH(CH₃)₂, C(CH₃)₃, cyclopropyl, and cyclobutyl.

6. The compound of claim 1, wherein ring C is selected from the group consisting of

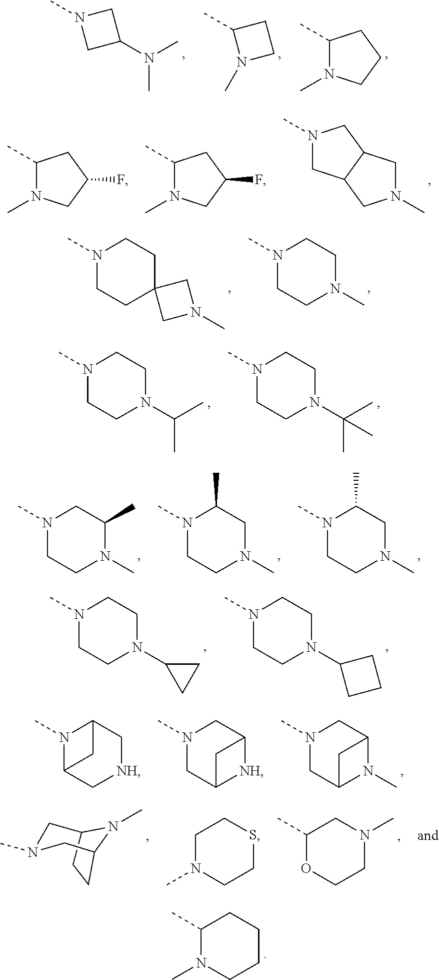

7. The compound of claim 1, wherein R¹ is selected from the group consisting of

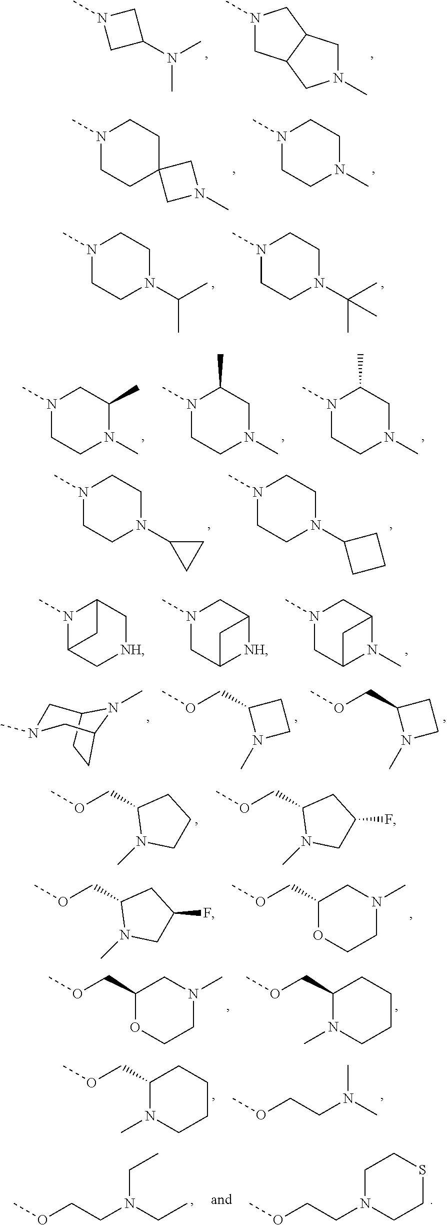

8. The compound of claim 1, wherein R^{2a}, R^{2b}, and R^{2c} are each independently selected from the group consisting of H and F.

9. The compound of claim 1, wherein R^{5a}, R^{5b}, and R^{5c} are each independently H.

10. The compound of claim 1, wherein R⁷ is selected from the group consisting of CH₃, CF₂H, CH₂CH₃, CH(CH₃)₂, and oxetanyl.

11. The compound of claim 1, wherein R^{8a} and R^{8b} are each independently H.

12. The compound of claim 1, wherein R^{6a} is selected from the group consisting of

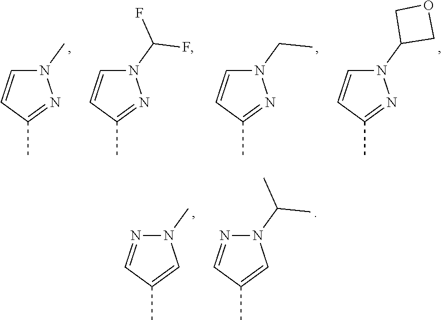

13. The compound of claim 1, wherein R⁹ is selected from the group consisting of CH₃, CH₂CH₃, CF₂H, CH₂CN, CH₂OCH₃, CH₂CF₂H, CH₂CF₃, CH₂(cyclopropyl), (CH₂)₂F, (CH₂)₂CN, (CH₂)₂OH, (CH₂)₂OCH₃, cyclopropyl, and oxetanyl.

14. The compound of claim 1, wherein R^{10a}, R^{10b}, R^{10c}, R^{10d}, and R^{10e}, if present, are each independently selected from the group consisting of H, CH₃, and O(CH₂)₂O(CH₂)₂OCH₃.

15. The compound of claim 1, wherein R^{6b} is selected from the group consisting of

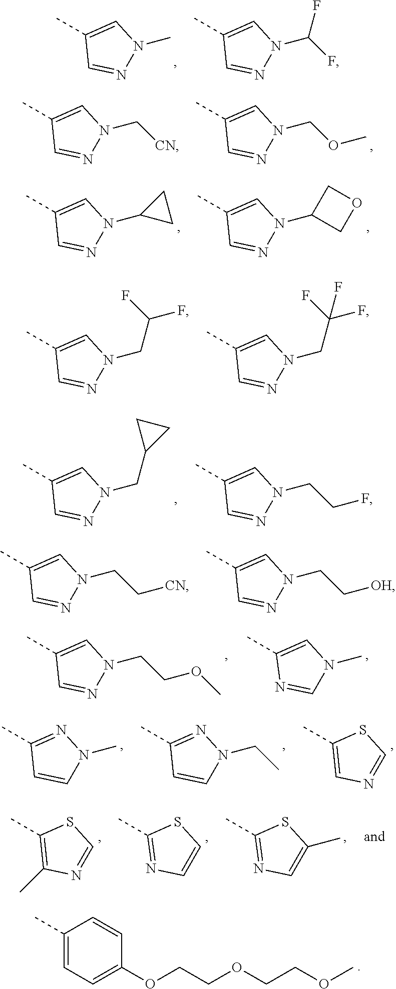

16. The compound of claim 1, which is selected from the group consisting of:
N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpyrrolidin-2-yl)methoxy)benzamide;
5-(3-(dimethylamino)azetidin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-5-((4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4S)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2S,4R)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2R,4R)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-5-(((2R,4S)-4-fluoro-1-methylpyrrolidin-2-yl)methoxy)-2-methylbenzamide;
N-(1-(3-(1-ethyl-1H-pyrazol-3-yl)-5-(1-methyl-1H-pyrazol-4-yl)phenyl)cyclopropyl)-2-methyl-5-(2-methyl-2,7-diazaspiro[3.5]nonan-7-yl)benzamide;
N-(1-(3-(1-ethyl-1H-pyrazol-3-yl)-5-(1-methyl-1H-pyrazol-4-yl)phenyl)cyclopropyl)-2-methyl-5-(5-methylhexahydropyrrolo[3,4-c]pyrrol-2(1H)-yl)benzamide;
4-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-4-fluoro-2-methyl-5-(4-methylpiperazin-1-yl)benzamide;
3-fluoro-N-(1-(3-(1-isopropyl-1H-pyrazol-4-yl)-5-(1-methyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-3-fluoro-2-methyl-5-(4-methylpiperazin-1-yl)benzamide;
5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-5-(4-isopropylpiperazin-1-yl)-2-methylbenzamide;
5-(4-cyclopropylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-fluoro-6-methyl-3-(4-methylpiperazin-1-yl)benzamide;
5-(4-(tert-butyl)piperazin-1-yl)-N-(1-(3-(1-ethyl-1H-pyrazol-3-yl)-5-(1-methyl-1H-pyrazol-4-yl)phenyl)cyclopropyl)-2-methylbenzamide;
5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(S)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
(R)-5-(2,4-dimethylpiperazin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(S)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
(R)-N-(1-(3-(1-isopropyl-1H-pyrazol-4-yl)-5-(1-methyl-1H-pyrazol-4-yl)phenyl)cyclopropyl)-2-methyl-5-((1-methylazetidin-2-yl)methoxy)benzamide;
N-(1-(3-(1-(cyclopropylmethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-ethyl-1H-pyrazol-3-yl)phenyl)cyclopropyl)-2-methyl-5-(4-methylpiperazin-1-yl)benzamide;
N-(1-(3-(1-(2,2-difluoroethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-cyclopropyl-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2,2,2-trifluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(thiazol-5-yl)phenyl)cyclopropyl)-2-methyl-5-(4-methylpiperazin-1-yl)benzamide;
5-(4-cyclobutylpiperazin-1-yl)-N-(1-(3-(1-ethyl-1H-pyrazol-3-yl)-5-(1-methyl-1H-pyrazol-4-yl)phenyl)cyclopropyl)-2-methylbenzamide;
N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((4-methylmorpholin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(R)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
(S)-N-(1-(3-(1-ethyl-1H-pyrazol-3-yl)-5-(1-methyl-1H-pyrazol-4-yl)phenyl)cyclopropyl)-2-methyl-5-((1-methylpiperidin-2-yl)methoxy)benzamide;
N-(1-(3-(1-ethyl-1H-pyrazol-3-yl)-5-(1-methyl-1H-pyrazol-4-yl)phenyl)cyclopropyl)-2-methyl-5-(2-thiomorpholinoethoxy)benzamide;
N-(1-(3,5-bis(1-ethyl-1H-pyrazol-3-yl)phenyl)cyclopropyl)-2-methyl-5-((1-methylazetidin-2-yl)methoxy)benzamide;
(S)-N-(1-(3,5-bis(1-ethyl-1H-pyrazol-3-yl)phenyl)cyclopropyl)-2-methyl-5-((1-methylazetidin-2-yl)methoxy)benzamide;
(R)-N-(1-(3,5-bis(1-ethyl-1H-pyrazol-3-yl)phenyl)cyclopropyl)-2-methyl-5-((1-methylazetidin-2-yl)methoxy)benzamide;
N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(S)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
(R)-N-(1-(3-(1-ethyl-1H-pyrazol-3-yl)-5-(1-isopropyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(2-fluoroethyl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(4-methylthiazol-5-yl)phenyl)cyclopropyl)-2-methyl-5-(4-methylpiperazin-1-yl)benzamide;
N-(1-(3-(1-ethyl-1H-pyrazol-3-yl)-5-(thiazol-2-yl)phenyl)cyclopropyl)-2-methyl-5-(4-methylpiperazin-1-yl)benzamide;
N-(1-(3-(1-ethyl-1H-pyrazol-3-yl)-5-(5-methylthiazol-2-yl)phenyl)cyclopropyl)-2-methyl-5-(4-methylpiperazin-1-yl)benzamide;
5-(2-(dimethylamino)ethoxy)-N-(1-(3-(1-ethyl-1H-pyrazol-3-yl)-5-(thiazol-5-yl)phenyl)cyclopropyl)-2-methylbenzamide;
5-(2-(dimethylamino)ethoxy)-N-(1-(3-(1-ethyl-1H-pyrazol-3-yl)-5-(thiazol-2-yl)phenyl)cyclopropyl)-2-methylbenzamide;
N-(1-(3-(1-(2-cyanoethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-(2-hydroxyethyl)-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(1-methyl-1H-pyrazol-4-yl)phenyl)cyclopropyl)-5-(4-methylpiperazin-1-yl)benzamide;
5-(2-(diethylamino)ethoxy)-N-(1-(3-(1-ethyl-1H-pyrazol-3-yl)-5-(1-methyl-1H-pyrazol-4-yl)phenyl)cyclopropyl)-2-methylbenzamide;
5-(3-(dimethylamino)azetidin-1-yl)-N-(1-(3-(1-isopropyl-1H-pyrazol-4-yl)-5-(1-methyl-1H-pyrazol-4-yl)phenyl)cyclopropyl)-2-methylbenzamide;
N-(1-(3-(1-ethyl-1H-pyrazol-3-yl)-5-(1-ethyl-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(4-methylpiperazin-1-yl)benzamide;
5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(R)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
(S)-5-(3-(dimethylamino)azetidin-1-yl)-N-(1-(3-(1-isopropyl-1H-pyrazol-4-yl)-5-(1-methyl-1H-pyrazol-4-yl)phenyl)ethyl)-2-methylbenzamide;
N-(1-(3-(1-ethyl-1H-pyrazol-3-yl)-5-(1-(oxetan-3-yl)-1H-pyrazol-4-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((S)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-((R)-1-(3-(1-ethyl-1H-pyrazol-3-yl)-5-(1-methyl-1H-pyrazol-4-yl)phenyl)ethyl)-2-methyl-5-(6-methyl-3,6-diazabicyclo[3.1.1]heptan-3-yl)benzamide;
N-(1-(3-(1-ethyl-1H-pyrazol-3-yl)-5-(1-(2-methoxyethyl)-1H-pyrazol-4-yl)phenyl)cyclopropyl)-2-methyl-5-(4-methylpiperazin-1-yl)benzamide;
N-(1-(3-(1-(cyanomethyl)-1H-pyrazol-4-yl)-5-(1-ethyl-1H-pyrazol-3-yl)phenyl)cyclopropyl)-2-methyl-5-(4-methylpiperazin-1-yl)benzamide;
N-(1-(3-(1-(difluoromethyl)-1H-pyrazol-3-yl)-5-(1-methyl-1H-pyrazol-4-yl)phenyl)cyclopropyl)-2-methyl-5-(4-methylpiperazin-1-yl)benzamide;
N-(1-(3-(1-isopropyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-imidazol-4-yl)phenyl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-5-yl)phenyl)cyclopropyl)-5-(4-methylpiperazin-1-yl)benzamide;
5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(R)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
(S)-5-(3,4-dimethylpiperazin-1-yl)-2-methyl-N-(1-(3-(1-methyl-1H-pyrazol-3-yl)-5-(1-methyl-1H-pyrazol-4-yl)phenyl)cyclopropyl)benzamide;
N-(1-(3-(1-(2-fluoroethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-(1-(3-(1-methyl-1H-pyrazol-3-yl)-5-(thiazol-2-yl)phenyl)cyclopropyl)-5-(4-methylpiperazin-1-yl)benzamide;
5-(3,6-diazabicyclo[3.1.1]heptan-3-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)cyclopropyl)-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-4-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-4-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
N-(1-(3-(1-ethyl-1H-pyrazol-3-yl)-5-(1-methyl-1H-pyrazol-3-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylazetidin-2-yl)methoxy)benzamide;
N-(1-(3,5-bis(1-methyl-1H-pyrazol-4-yl)phenyl)cyclopropyl)-2-methyl-5-((1-methylazetidin-2-yl)methoxy)benzamide;
(S)-N-(1-(3,5-bis(1-methyl-1H-pyrazol-4-yl)phenyl)cyclopropyl)-2-methyl-5-((1-methylazetidin-2-yl)methoxy)benzamide;
(R)-N-(1-(3,5-bis(1-methyl-1H-pyrazol-4-yl)phenyl)cyclopropyl)-2-methyl-5-((1-methylazetidin-2-yl)methoxy)benzamide;
N-(1-(3-(1-ethyl-1H-pyrazol-3-yl)-5-(1-(oxetan-3-yl)-1H-pyrazol-3-yl)phenyl)cyclopropyl)-2-methyl-5-(4-methylpiperazin-1-yl)benzamide;
N-(1-(3-(1-ethyl-1H-pyrazol-3-yl)-5-(1-methyl-1H-pyrazol-4-yl)phenyl)cyclopropyl)-2-methyl-5-(3-methyl-3,6-diazabicyclo[3.1.1]heptan-6-yl)benzamide;
2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(S)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
(R)-2-methyl-N-(1-(3-(1-methyl-1H-pyrazol-3-yl)-5-(1-methyl-1H-pyrazol-4-yl)phenyl)cyclopropyl)-5-((1-methylpyrrolidin-2-yl)methoxy)benzamide;
N-(1-(3-(1-ethyl-1H-pyrazol-4-yl)-5-(1-methyl-1H-pyrazol-3-yl)phenyl)cyclopropyl)-2-methyl-5-(4-methylpiperazin-1-yl)benzamide;
N-(1-(3-(1-(methoxymethyl)-1H-pyrazol-4-yl)-5-(1-methyl-1H-pyrazol-3-yl)phenyl)cyclopropyl)-2-methyl-5-(4-methylpiperazin-1-yl)benzamide;
2-methyl-N-((R)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1R,5S)-8-methyl-3,8-diazabicyclo[3.2.1]octan-3-yl)benzamide;
2-methyl-N-((R)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-((S)-1-(3-(1-methyl-1H-pyrazol-3-yl)-5-(1-methyl-1H-pyrazol-4-yl)phenyl)ethyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1R,5S)-8-methyl-3,8-diazabicyclo[3.2.1]octan-3-yl)benzamide;
2-methyl-N-(1-(3-(1-methyl-1H-pyrazol-3-yl)-5-(1-methyl-1H-pyrazol-4-yl)phenyl)cyclopropyl)-5-((1S,5R)-8-methyl-3,8-diazabicyclo[3.2.1]octan-3-yl)benzamide;
2-methyl-N-(1-(3-(1-methyl-1H-pyrazol-3-yl)-5-(5-methylthiazol-2-yl)phenyl)cyclopropyl)-5-(4-methylpiperazin-1-yl)benzamide;
2-methyl-N-(1-(3-(1-methyl-1H-pyrazol-3-yl)-5-(4-methylthiazol-5-yl)phenyl)cyclopropyl)-5-(4-methylpiperazin-1-yl)benzamide;
5-(3,6-diazabicyclo[3.1.1]heptan-6-yl)-N-(1-(3-(1-ethyl-1H-pyrazol-3-yl)-5-(1-methyl-1H-pyrazol-4-yl)phenyl)cyclopropyl)-2-methylbenzamide;
2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(R)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
(S)-2-methyl-N-(1-(3-(1-methyl-1H-pyrazol-3-yl)-5-(1-methyl-1H-pyrazol-4-yl)phenyl)ethyl)-5-(4-methylpiperazin-1-yl)benzamide;
2-methyl-N-(1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((R)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
2-methyl-N-((S)-1-(3-(1-methyl-1H-pyrazol-3-yl)-5-(1-methyl-1H-pyrazol-4-yl)phenyl)ethyl)-5-(6-methyl-3,6-diazabicyclo[3.1.1]heptan-3-yl)benzamide;
N-(1-(4′-(2-(2-methoxyethoxy)ethoxy)-5-(1-methyl-1H-pyrazol-3-yl)-[1,1′-biphenyl]-3-yl)cyclopropyl)-2-methyl-5-(4-methylpiperazin-1-yl)benzamide;
N-(1-(3,5-bis(1-methyl-1H-pyrazol-4-yl)phenyl)cyclopropyl)-2-methyl-5-(6-methyl-3,6-diazabicyclo[3.1.1]heptan-3-yl)benzamide;
2-methyl-N-(1-(3-(1-methyl-1H-pyrazol-3-yl)-5-(1-(oxetan-3-yl)-1H-pyrazol-4-yl)phenyl)cyclopropyl)-5-(4-methylpiperazin-1-yl)benzamide;
N-(1-(3-(1-ethyl-1H-pyrazol-3-yl)-5-(1-(methoxymethyl)-1H-pyrazol-4-yl)phenyl)cyclopropyl)-2-methyl-5-(6-methyl-3,6-diazabicyclo[3.1.1]heptan-3-yl)benzamide; and
N-(1-(3-(1-(2-methoxyethyl)-1H-pyrazol-4-yl)-5-(1-methyl-1H-pyrazol-3-yl)phenyl)cyclopropyl)-2-methyl-5-(4-methylpiperazin-1-yl)benzamide.

17. A pharmaceutical composition comprising at least one compound of claim 1 and a pharmaceutically acceptable carrier.

18. A method for promoting an antiviral effect in a subject, the method comprising administering at least one compound of claim 1 or a pharmaceutical composition thereof.

19. A method for inhibiting a papain-like protease in a subject, the method comprising administering to the subject at least one compound of claim 1 or a pharmaceutical composition thereof.

20. A method for treating, preventing, or ameliorating a viral infection in a subject, the method comprising administering to the subject at least one compound of claim 1 or a pharmaceutical composition thereof.
